(19) United States
(12) Patent Application Publication
(10) Pub. No.: US 2024/0225971 A1
(43) Pub. Date: Jul. 11, 2024

(54) 1,3-BUTYLENE GLYCOL COMPOSITIONS AND METHODS OF USE THEREOF

(71) Applicant: Genomatica, Inc., San Diego, CA (US)

(72) Inventor: Kyle HUSTON, San Diego, CA (US)

(21) Appl. No.: 18/569,956

(22) PCT Filed: Jun. 10, 2022
(86) PCT No.: PCT/US2022/033061

§ 371 Date: Dec. 13, 2023

Related U.S. Application Data
(60) Provisional application No. 63/210,393, filed on Jun. 14, 2021.

(51) Int. Cl. A61K 8/34; A61Q 13/00

(52) U.S. Cl. CPC A61K 8/345; A61Q 13/00

(57) ABSTRACT

The disclosure provides embodiments directed to a composition comprising a 1,3-butylene glycol (1,3-BG; such as but not limited to, bioBG) and one or more fragrance ingredients, which form a fragrance component of the composition. 1,3-BG demonstrates exemplary qualities and characteristics of being natural, sustainable, and substantially non-odorous, for use in fragrance compositions. Methods of using or applying such 1,3-BG fragrance compositions provide a cost-efficient, sustainable composition, since 1,3-BG does not mask or alter the fragrance notes of the fragrance ingredients allowing for the use of smaller quantities of fragrance ingredients. The 1,3-BG fragrance compositions of the disclosure also improve fragrance performance and reduces greenhouse gases, while avoiding any irritation, sensitization, or allergic reaction in humans.

### CROSS-REFERENCE TO RELATED APPLICATIONS

This application claims the benefit of U.S. Provisional Patent Application No. 63/210,393, filed Jun. 14, 2021, the entire contents of which are incorporated by reference herein.

### FIELD OF DISCLOSURE

The present disclosure is generally related to uses and compositions of 1,3-butylene glycol, where 1,3-BG is utilized in fragrance compositions.

### BACKGROUND

Fragrances are a combination of chemicals that provide distinct scents of fragrance ingredients. Fragrance compositions may also contain solvents, stabilizers, preservatives, UV-absorbers, and the like, in addition to the scent-providing fragrance ingredients. However, many of these additions are derived from petroleum or natural raw materials.

Demand for sustainable personal care products is on the rise. A 2020 survey found that more than a third of Americans are willing to spend more money for sustainable products. While sustainable soaps, lotions and shampoos are increasingly available, the industry continues to search for new, sustainable ingredients for perfumes and fragrances.

Butylene glycol (BG), a colorless organic alcohol, is used as an organic solvent for food flavoring agents, a co-monomer for production of polyurethane and polyester resins, and is widely employed as a hypoglycemic agent. Additionally, BG may be found in personal care products to increase moisture retention (as a humectant) and improve texture. BG may also be used to evenly disperse plant extracts, flavors, and fragrances.

The compound BG (also known as 1,3-butylene glycol; 1,3-BG; 1,3-butanediol; 1,3-BDO) is a four carbon diol (C₄H₁₀O₂) traditionally produced from petroleum-derived acetylene through its hydration. The resulting acetaldehyde is then converted to 3-hydroxybutyraldehdye which is subsequently reduced to form a petroleum-derived 1,3-butylene glycol. However, this conventional petrochemical process begins with acetaldehyde, which is a mutagen and carcinogen.

Many perfume makers have been criticized for using potentially hazardous chemicals, some of which are derived from petroleum, termed “petrochemicals.” Petrochemical plants have also been a source of toxicity to the environment as well as individuals. For example, petrochemicals in the form of oil spills poison plant and ocean life. When petrochemicals are burned, ash, carbon, nitrogen, and sulfur are released into the atmosphere in the form of smog, pollution, and acid rain. Greenhouse gases, including carbon dioxide, ozone, methane, nitrous oxide, chlorofluorocarbons, etc., are also created when petrochemicals are burned. Populations living near industrial sites manufacturing or using petrochemicals have been found to have adverse health effects, including but not limited to, eye irritation, respiratory issues, nervous system effects, and cancer resulting from exposure to these industrial pollutants. Furthermore, while petroleum and natural gas were once cheap and plentiful, these raw materials are increasingly expensive and in diminishing supply.

One standard of diluents in the fragrance industry is dipropylene glycol (DPG), which dilutes otherwise insoluble ingredients. However, DPG is derived from petroleum, which is not ideal for use in natural fragrances. Other diluents used in fragrance compositions include benzyl benzoate (BB) and benzyl alcohols, which are aroma chemicals or chemical substances that impart a scent or odor. These aroma chemicals may be synthetic or derived from natural ingredients. Yet, there are problems with natural fragrance ingredients, including difficulties blending with other fragrance ingredients, weak or faint scents, difficulty replicating scents, may cause allergies or skin sensitivities, or combinations of these issues.

Therefore, there is a need to develop an alternative to petroleum-derived conventional materials that are cost-efficient fragrance compositions using safe, natural, and sustainable additives without inhibiting scents or fragrance notes of the fragrance ingredients. Embodiments of the present disclosure satisfy this need and provide related advantages as well.

### SUMMARY

In accordance with the foregoing objectives and others, the present disclosure provides in one aspect, a composition, comprising: (a) a 1,3-butylene glycol (1,3-BG), such as a bioderived 1,3-butylene glycol (bioBG); and (b) one or more fragrance ingredients, where the components of (a) and (b) form a fragrance component, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur.

Another aspect of the disclosure provides a composition comprising a 1,3-BG, such as a bioBG, and one or more fragrance ingredients, where the 1,3-BG or bioBG is a racemate or mixture of: (R)-1,3-BG and (S)-1,3-BG. In other aspects, the composition comprises a mixture of 75% or greater of (R)-1,3-BG. A further aspect is directed to the composition comprising a mixture of 25% or less of (S)-1,3-BG. Some aspects provide for such a composition, where the one or more fragrance ingredients comprises (R)-1,3-BG.

Yet another aspect provides a composition of the disclosure, where the 1,3-BG or bioBG is a microbial growth inhibitor and a preservative. In one aspect, 1,3-BG and the bioBG that is a natural, sustainable diluent, are substantially non-odorous and do not react or alter the scents or notes produced by fragrance ingredients. Further aspects provide 1,3-BG or bioBG that improves expression of one or more fragrance ingredients, or their odor or notes, in a composition comprising the same. Another aspect is directed to 1,3-BG diluent, such as bioBG, where the 1,3-BG diluent does not mask or suppress the scent or odor of the one or more fragrance ingredients, which allows for the reduction of the amount of fragrance ingredients. In yet another aspect of the disclosure, the one or more fragrance ingredients is selected from the characteristics consisting of: a molecular weight greater than or equal to 90 g/mol, less than or equal to 270 g/mol, or in a range of 90 g/mol to 270 g/mol; a log P value greater than or equal to 0.5, less than or equal to 7, or in a range of 0.5 to 7; a mixture of polar and non-polar fragrance ingredients; a mixture of more polar fragrance ingredients than non-polar fragrance ingredients; polar fragrance ingredients; a mixture of natural and synthetic fragrance ingredients; and synthetic fragrance ingredients, or combinations thereof.

The composition in a further aspect of the disclosure is directed to a fragrance component comprising one or more fragrance ingredients present in an amount greater than or equal to 0.001% by weight relative to the total % by weight of fragrance component or composition. Another aspect is directed to one or more fragrance ingredients present in an amount less than or equal to 60% by weight relative to the total % by weight of fragrance component of composition. In yet a further aspect, the one or more fragrance ingredients is present in an amount of 0.015% by weight to 60% by weight relative to the total % by weight of fragrance component of composition.

In one aspect, the disclosure provides a composition comprising: (a) a 1,3-butylene glycol or bioderived 1,3-butylene glycol (bioBG); and (b) one or more fragrance ingredients, where the components of (a) and (b) form a fragrance component, where the composition or fragrance component is selected from the group of properties consisting of: non-irritating, non-sensitizing, non-allergenic, non-greasy, and any combinations thereof.

Another aspect of the disclosure is directed to a method, comprising applying to skin of a subject, a composition comprising (a) a 1,3-butylene glycol or bioderived 1,3-butylene glycol (bioBG); and (b) one or more fragrance ingredients, where the components of (a) and (b) form a fragrance component, where the composition is a fragrance composition. A further aspect of the fragrance composition provides one or more fragrance ingredients selected from the group of characteristics consisting of: polar, synthetic, excellent solubility, clear clarity, moderate to bright headspace, and combinations thereof.

One aspect of the disclosure provides a use of 1,3-butylene glycol, such as bioderived 1,3-butylene glycol, as a diluent in a fragrance composition, where the fragrance composition further comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur, and the 1,3-BG, such as bioBG, and one or more fragrance ingredients form a fragrance component.

### BRIEF DESCRIPTION OF FIGURES

FIG. 1 shows the odor performance value (left axis) and bioderived 1,3-butylene glycol (bioBG) solubility percentage (right axis) for each of the fragrance ingredients at 10% on the x-axis.

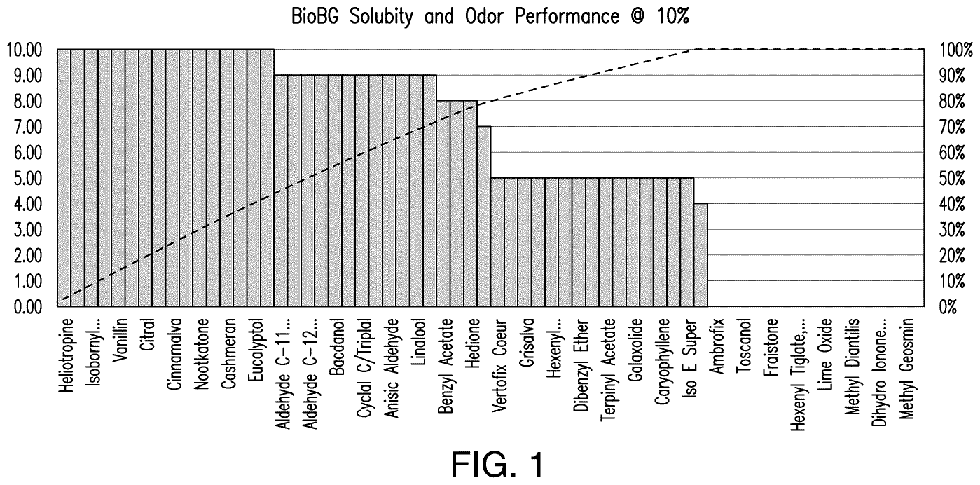

FIG. 2 demonstrates the odor performance value (left axis) and dipropylene glycol (DPG) solubility percentage (right axis) for each of the fragrance ingredients on the x-axis at 10%.

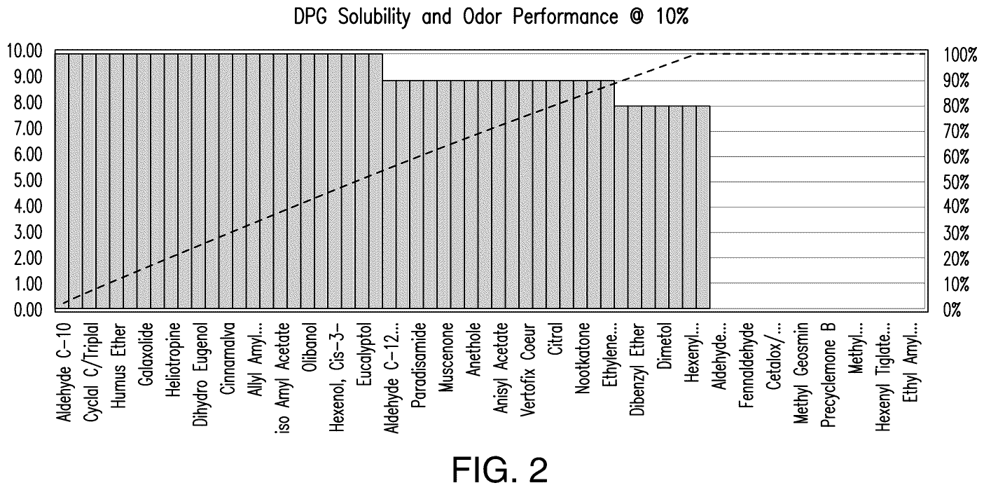

FIG. 3 presents the odor performance value (left axis) and benzyl benzoate (BB) solubility percentage (right axis) for each of the fragrance ingredients on the x-axis at 10%.

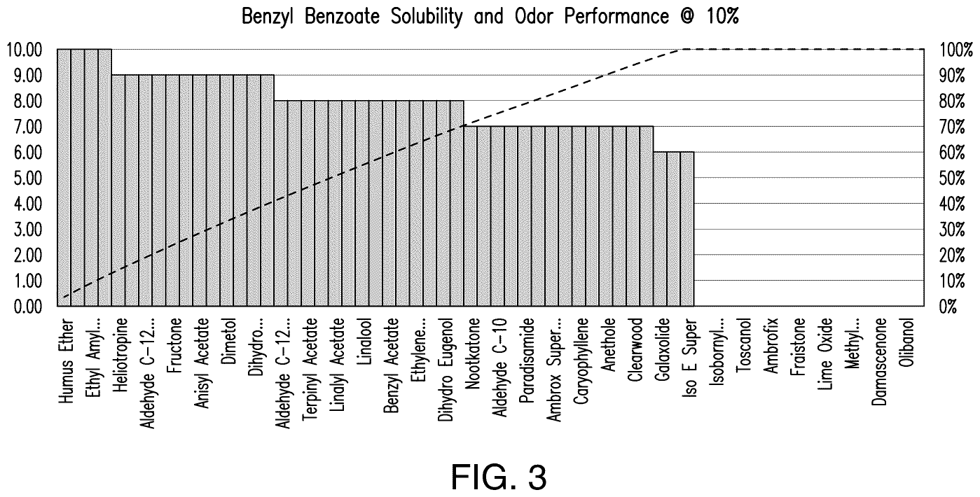

FIG. 4 illustrates the odor performance value (left axis) and isopropyl myristate (IPM) solubility percentage (right axis) for each of the fragrance ingredients on the x-axis at 10%.

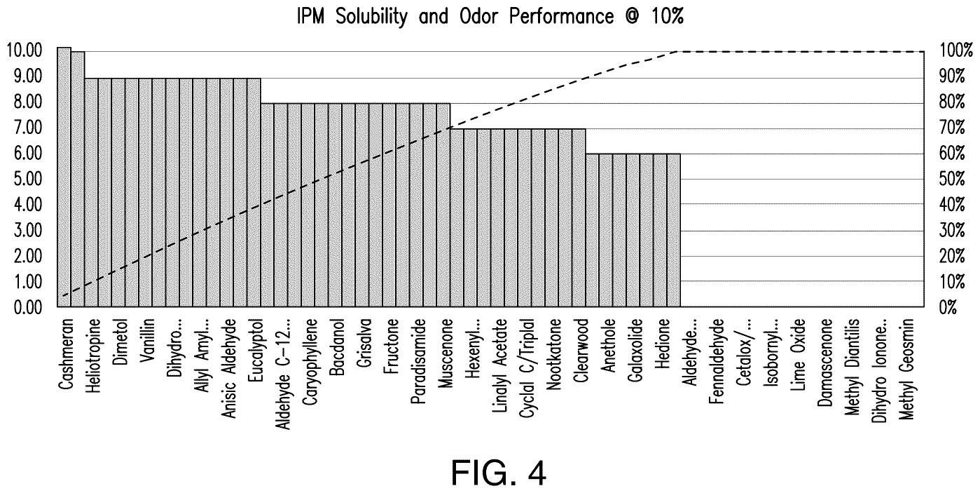

FIG. 5 shows the odor performance value (left axis) and bioBG solubility percentage (right axis) for each of the fragrance ingredients on the x-axis at an overall % (i.e., the average of all dilutions of 0.01%, 0.1%, 1%, and 10%).

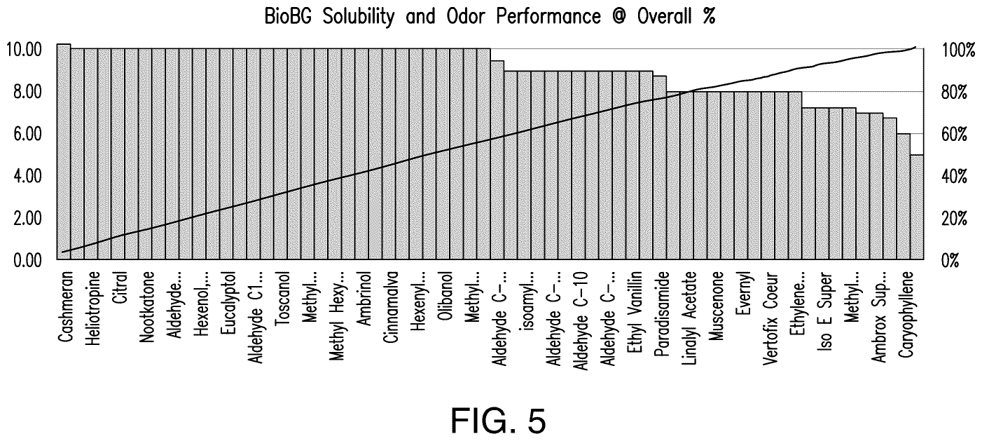

FIG. 6 demonstrates the odor performance value (left axis) and DPG solubility percentage (right axis) for each of the fragrance ingredients on the x-axis at an overall % (i.e., the average of all dilutions of 0.01%, 0.1%, 1%, and 10%).

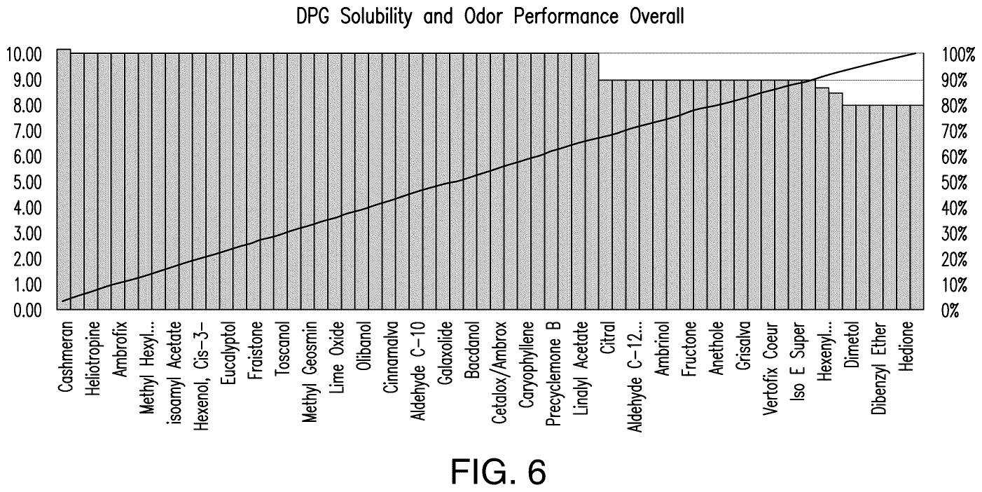

FIG. 7 presents the odor performance value (left axis) and BB solubility percentage (right axis) for each of the fragrance ingredients on the x-axis at an overall % (i.e., the average of all dilutions of 0.01%, 0.1%, 1%, and 10%).

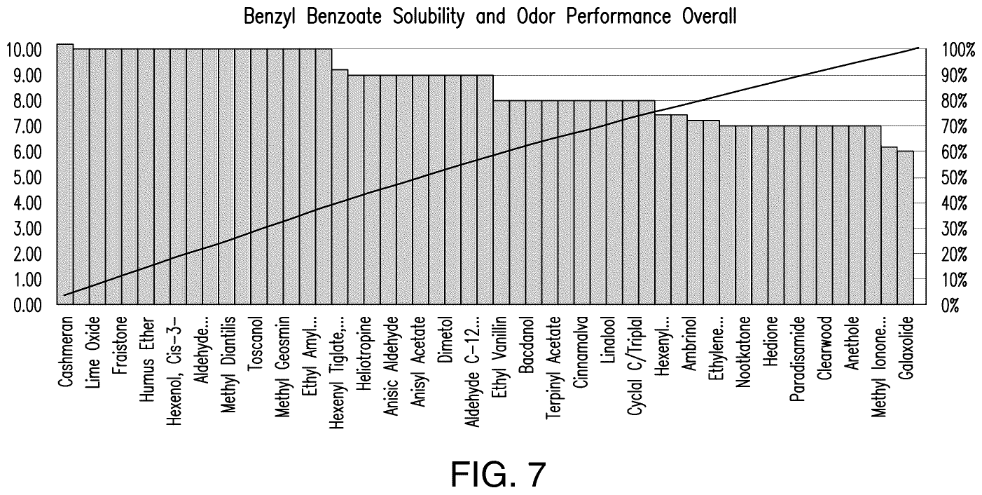

FIG. 8 illustrates the odor performance value (left axis) and IPM solubility percentage (right axis) for each of the fragrance ingredients on the x-axis at an overall % (i.e., the average of all dilutions of 0.01%, 0.1%, 1%, and 10%).

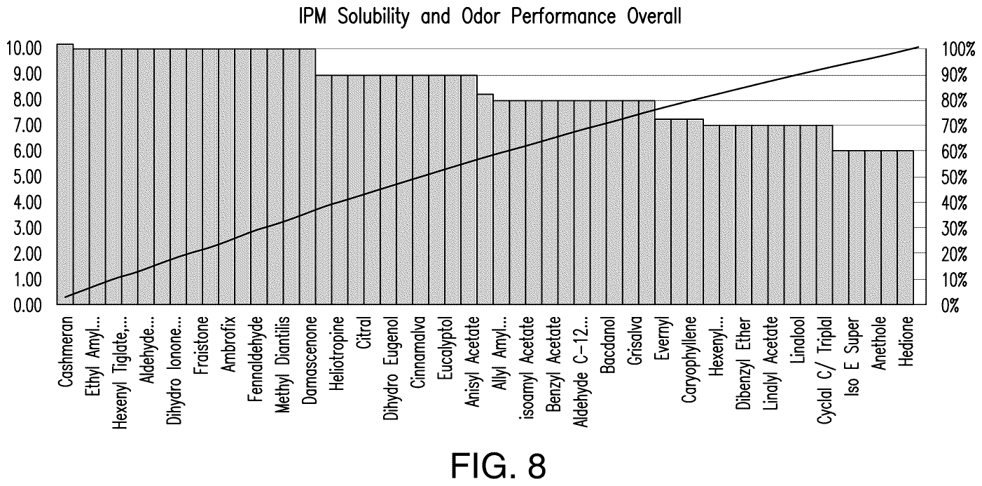

FIG. 9 shows a log P line fit plot for bioBG. The log P value (x-axis) is compared to the performance at 1% solubility (y-axis), where the solubility of 105 common fragrance ingredients diluted in bioBG was visually inspected and assigned a performance rating of (1) poor; (2) moderate; (3) good; and (4) excellent. The predicted value at 1% solubility (square) and the actual value at 1% (diamond).

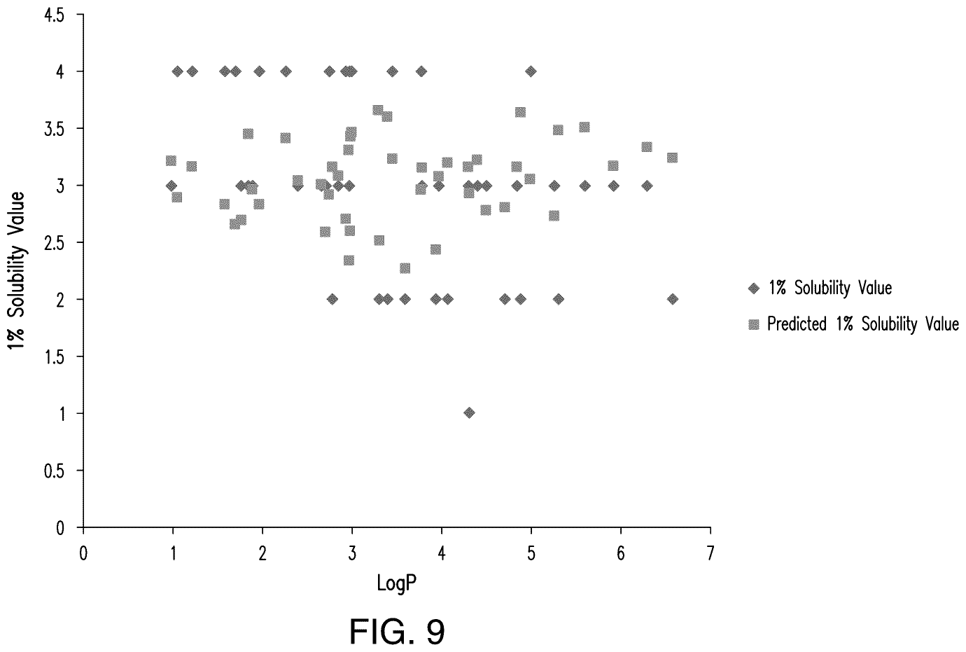

FIG. 10 demonstrates a molecular weight line fit plot for bioBG. The molecular weight value (x-axis) is compared to the performance at 1% solubility (y-axis), where the solubility of 105 common fragrance ingredients diluted in bioBG was visually inspected and assigned a performance rating of (1) poor; (2) moderate; (3) good; and (4) excellent. The legend shows the predicted value at 1% solubility (square) and the actual value at 1% (diamond).

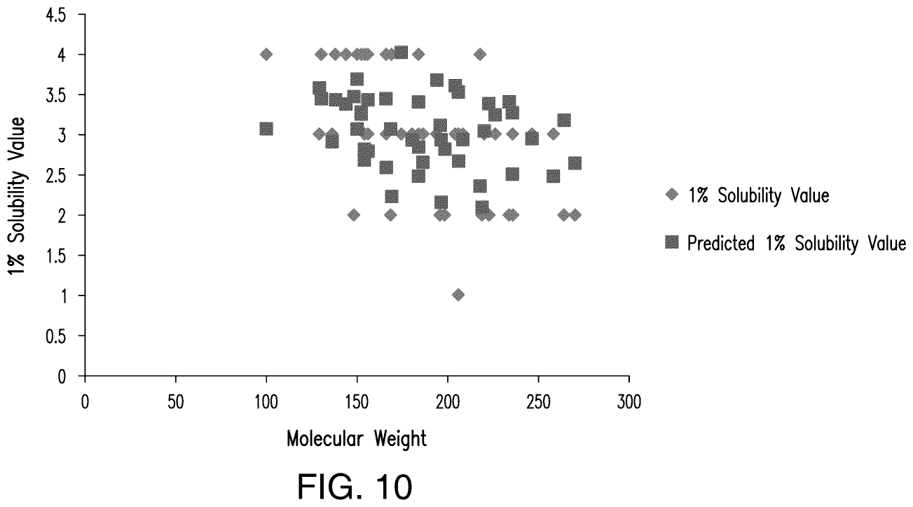

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component (e.g., fragrance ingredient) relative to the entire weight of the composition, unless otherwise defined.

Embodiments of the disclosure are directed to cosmetics, personal care products, and household, commercial, or industrial products, such as but not limited to, any compositions containing a fragrance component, e.g., fragrances or fragrance compositions, where the fragrance component comprises one or more fragrance ingredients and a 1,3-butylene glycol (synonymous and used interchangeably with BG, 1,3-BG; 1,3-butanediol; 1,3-BDO; butane-1,3-diol; etc.), such as a bioderived 1,3-butylene glycol. CAS No. 107-88-0 is assigned to 1,3-BG having a chemical formula of C₄H₁₀O₂. There are various methods for producing 1,3-butylene glycol. For example, U.S. Pat. No. 6,376,725, which is incorporated herein by reference in its entirety, describes methods of liquid phase hydrogenation of acetaldol (3-hydroxybutanal or aldol) with a Raney nickel catalyst resulting in 1,3-butylene glycol. Another method of producing 1,3-butylene glycol is described as having three steps: aldol condensation of acetaldehyde to aldoxane, then decomposition of the aldoxane resulting in paraldol, which is then hydrogenated to finally produce 1,3-butylene glycol (as described in, e.g., U.S. Pat. Nos. 5,345,004; 5,583,270, which are incorporated herein by reference in their entirety). One example of a 1,3-BG is a sustainable, natural bioderived 1,3-BG (bioBG), which may be produced by non-naturally occurring microorganisms that express genes encoding enzymes that catalyze bioderived 1,3-BG production. The methods and compositions for producing bioderived 1,3-BG through culturing of non-naturally occurring microbial organisms are described in, for example, WO 2010/127319 (e.g., Examples I-III), WO 2011/071682 (e.g., Examples I, III), and WO 2012/177619 (e.g., Examples I-IV, VIII), which disclose methods, vectors, strains, and culturing materials and conditions for producing bioderived 1,3-BG; and WO 2018/183628 (e.g., Example 1) and WO 2018/183664, which specifically disclose engineered organisms and enzymes producing bioderived 1,3-BG, as well as characterization of bioderived 1,3-BG, each of which is incorporated by reference in its entirety. The compositions, methods, and uses disclosed here, in any or all embodiments, are directed to 1,3-BG, which may include any type or form of 1,3-BG, including but not limited to 1,3-BG; bioBG; mixtures of 1,3-BG and bioBG; (R)-1,3-BG; (S)-1,3-BG; mixtures of (R)-1,3-BG and (S)-1,3-BG; or any combinations or mixtures of the aforementioned.

As used herein, the term “non-naturally occurring” when used in reference to a microbial organism or microorganism of the disclosure is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, but are not limited to, coding regions and functional fragments thereof, for heterologous, homologous, or both heterologous and homologous polypeptides for the referenced species. Additional modifications include, for example, non-coding regulatory regions in which the modifications alter expression of a gene or operon. Exemplary metabolic polypeptides include enzymes or proteins within a 1,3-butanediol biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof.

These microorganisms may be supplied a source of carbon via any carbohydrate source. Such sources include, but are not limited to, sugars (e.g., hexoses: glucose, fructose, galactose, mannose; pentoses: xylose, arabinose) and starches. Plant biomass, an exemplary carbohydrate source, is a type of renewable organic material from plants that can be converted into chemicals, biofuels, and other materials. Non-limiting carbohydrate sources include renewable plant biomasses and feedstocks, such as but not limited to, cellulosic biomass, hemicellulosic biomass, and lignin feedstocks or portions of feedstocks.

As used herein, the term “bioderived” means derived from or synthesized by a biological organism and can be considered a renewable resource since a bioderived product can be generated by a biological organism. Such a biological organism, in particular, the microbial organisms of the disclosure described here, can utilize feedstock or biomass, such as, sugars or carbohydrates obtained from an agricultural, plant, bacterial, or animal source. Alternatively, the biological organism can utilize atmospheric carbon. As used here, the term “biobased” means a product as described here that is composed of, in whole or in part, a bioderived compound of the disclosure. A biobased or bioderived product of the disclosure (e.g., bioderived 1,3-butylene glycol or bioBG) is not, and is in contrast to, a “petroleum-derived product,” where such a petroleum-derived product is derived from or synthesized from petroleum or a petrochemical feedstock. A bioderived 1,3-butylene glycol used here is a natural product as measured by the International Organization for Standardization (ISO). ISO 16128-1:2016 provides definitions for organic and natural cosmetic ingredients, and ISO 16128-2:2017 provides guidelines and criteria for determining natural, organic, or natural and organic origins of cosmetic ingredients and products.

In some embodiments, the 1,3-butylene glycol (1,3-BG) including bioderived 1,3-butylene glycol (bioBG) of the disclosure is, for example, a pure diluent, both visually and with respect to having little or few undesirable contaminants. In another embodiment of the disclosure, the 1,3-BG or bioBG is substantially or essentially non-odorous (and does not mask an odor, add an odor, or alter a fragrance profile of the one or more fragrance ingredients of a composition) yet has also been perceived by a master perfumer as having a sweet odor and a slight green note in the background. Further embodiments provide an essentially non-odorous 1,3-BG or bioBG having an improved odor characteristic compared to petroleum-derived (dipropylene glycol) or other natural diluents (e.g., benzyl benzoate; isopropyl myristate). Furthermore, 1,3-BG or bioBG has several properties advantageous for consumer use or application. For example, 1,3-BG or bioBG is non-irritating, non-sensitizing, non-allergenic, non-greasy, or has any combinations of the aforementioned properties. Compositions of the disclosure comprising 1,3-BG or bioBG are also non-irritating, non-sensitizing, non-allergenic, non-greasy, or any combinations of the aforementioned properties.

Another embodiment of the disclosure provides 1,3-BG, including bioderived 1,3-butylene glycol, comprising (R)-1,3-butylene glycol (CAS No. 6290-03-5); (S)-1,3-butylene glycol (CAS No. 24621-61-2); or combinations or mixtures of (R)-1,3-butylene glycol and (S)-1,3-butylene glycol. In other embodiments, the 1,3-butylene glycol consists essentially of: (R)-1,3-butylene glycol; (S)-1,3-butylene glycol; or combinations or mixtures of (R)-1,3-butylene glycol and (S)-1,3-butylene glycol. In another embodiment, the 1,3-BG or bioBG is a racemate, or a mixture of (R)- and (S)-enantiomers of 1,3-BG. Some embodiments provide for a bioBG racemate having more (R)-1,3-BG than (S)-1,3-BG. Further embodiments provide for 1,3-BG or bioBG comprising or consisting essentially of: (R)-1,3-butylene glycol. Some embodiments provide for a 1,3-BG or bioBG racemate having more (R)-1,3-BG than (S)-1,3-BG. In another embodiment, the 1,3-BG, including bioBG, comprises (R)-1,3-BG, where (R)-1,3-butylene glycol is present in 1,3-BG or bioBG in more than or equal to 75% (e.g., 80%, 85%, 90%, 95%; 96%; 97%; 98%; 99%; 99.5%; 99.9%; 100%); where (R)-1,3-butylene glycol is present in 1,3-BG or bioBG in less than or equal to 100% (e.g., 99.8%, 99.3%, 98.5%, 97.5%, 96.5%, 95.5%, 93%, 87%, 83%, 77%); or (R)-1,3-butylene glycol, where (R)-1,3-butylene glycol is present in 1,3-BG or bioBG in 75%-100% (e.g., 80%-99%; 85%-95%; 87%-93%) as detected by mechanical separation, reaction with enzymes, formation of diastereomers, or chromatographic methods (e.g., chiral chromatography, gas chromatography, GC-MS, LC-MS, MD-GC, TLC). In another embodiment, a 1,3-BG or bioBG comprises (S)-1,3-butylene glycol, where (S)-1,3-butylene glycol is present in 1,3-BG or bioBG in more than or equal to 0% (e.g., 0.001%, 0.01%, 0.05%, 0.1%, 0.15%, 0.2%, 0.5%, 1%, 2%, 3%, 4%, 5%, 10%, 20%, 25%, 30%); (S)-1,3-butylene glycol, where (S)-1,3-butylene glycol is present in 1,3-BG or bioBG in less than or equal to 25% (e.g., 20%, 15%, 12%, 8%, 5.5%, 4.5%, 3.5%, 2.5%, 1.5%, 1.2%, 0.8%, 0.3%, 0.1%, 0.08%, 0.02%, 0.005%, 0.001%); or (R)-1,3-butylene glycol, where (R)-1,3-butylene glycol is present in 1,3-BG or bioBG in 0%-25% (e.g., 1%-22%; 2%-12%; 3%-10%; 4%-8%).

The 1,3-butylene glycol or bioderived butylene glycol (bioBG), provided here can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, the 1,3-butylene glycol may exist in various stereoisomeric forms. “Stereoisomers” are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system). Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared or obtained as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least, for example, 60%, 65%, 70%, 75%, 80%, 85%, 90%, 95%, 99%, or 99.9% by weight relative to the other stereoisomers. When a single enantiomer is named or depicted by structure, the depicted or named enantiomer is at least, for example, 60%, 70%, 80%, 90%, 99%, or 99.9% by weight optically pure. When a single diastereomer is named or depicted by structure, the depicted or named diastereomer is at least, for example, 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least, for example, 60%, 70%, 80%, 90%, 99%, or 99.9% by mole fraction pure. When a single diastereomer is named or depicted by structure, the depicted or named diastereomer is at least, for example, 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either stereoisomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

In some embodiments, the combined 1,3-BG (e.g., bioBG) diluent and one or more fragrance ingredients portion of the composition of the disclosure, forms a fragrance component. The dilution or percent solutions of fragrance ingredient(s) in diluent or carrier may be in the form of weight/volume % (wt/vol % or w/v %), weight/weight % (wt/wt % or w/w %), or volume/volume % (vol/vol % or v/v %), where the amount (weight or volume) of a solute (e.g., fragrance ingredient(s) is expressed as a percentage of the total solution or fragrance component weight or volume (i.e., fragrance ingredient(s) and diluent). The amount of fragrance ingredient in 1,3-BG, including bioBG diluent or carrier in total solution or fragrance component of the disclosure include percentages of greater than or equal to 0.01% by weight (e.g., 0.06%; 0.11%; 0.16%; 0.21%; 0.26%; 0.31%; 0.36%; 0.41%; 0.46%; 0.51%; 0.56%; 0.61%; 0.66%; 0.71%; 0.76%; 0.81%; 0.86%; 0.91%; 0.96%; 1%; 1.05%; 1.25%; 1.5%; 1.75%; 2%; 2.5%; 3%; 3.5%; 4%; 4.5%; 5%; 5.5%; 6%; 6.5%; 7%; 7.5%; 8%; 8.5%; 9%; 9.5%; 10%; 10.5%; 15%; 20%; 25%; 30%; 35%; 40%; 45%; 50%; 55%; 60%); less than or equal to 50% (e.g., 45%; 40%; 35%; 30%; 25%; 20%; 15%; 10%; 9.5%; 8%; 7%; 6%; 5%; 4%; 3%; 2%; 1.75%; 1.25%; 1%; 0.75%; 0.25%; 0.17%; 0.12%; 0.07%; 0.02%; 0.01%); or percentages in ranges of 0.01%-50% (e.g., 0.05%-45%; 0.1%-30%; 1%-20%; 3%-15%; 5%-10%).

Some embodiments of the disclosure provide fragrance compositions comprising fragrance ingredients diluted in a diluent or carrier comprising 1,3-BG, such as, but not limited to, bioderived 1,3-BG. Non-limiting examples of fragrance compositions comprising one or more fragrance ingredients in a diluent at an amount by weight or volume of fragrance ingredient(s) to weight or volume of total solution or composition may typically include: Eau Fraiche: 1%-3%; Eau de Cologne: 3%-8%; Eau de Toilette: 5%-10%; Eau de Parfum: 10%-20%; Extrait or Parfum: 15%-30%; Solid Perfume (Parfum Solide): 20%-45%. Since, the diluent 1,3-BG, including bioBG, is substantially or essentially non-odorous, and does not mask or suppress an odor, add an odor, or alter a fragrance profile of the one or more fragrance ingredients of a composition of the disclosure, the amount of the one or more fragrance ingredients may be reduced, yet still provide a strong level of odor or scent without affecting the fragrance profile. An advantage of using less fragrance ingredients is the cost savings to the manufacturer, and potentially to the consumer, since the process of obtaining the fragrance ingredients and/or the fragrance ingredients themselves is costly.

One embodiment provides fragrance compositions comprising one or more fragrance ingredients of the disclosure and 1,3-BG (e.g., bioBG) diluent, where the percentages of total fragrance ingredients are less than what is typically used in compositions containing a fragrance, such as those identified above. Additional compositions or fragrance compositions include, but are not limited to, aromatherapy compositions for baths, chest rubs, compresses, facial steams, massage oils, respiratory inhalations, room sprays, candles, clothing/linen sprays; stationery sprays; personal care compositions such as body/facial sprays, facial serums, creams, lotions, deodorant/anti-perspirants, shampoos, conditioners, body washes; or any compositions that utilize fragrance, where the fragrance ingredient(s) in 1,3-BG or bioderived 1,3-BG is in an amount sufficient to obtain the desired fragrance level and fragrance profile.

In one embodiment, the composition of the disclosure comprises (a) 1,3-BG (e.g., a bioderived 1,3-BG); and (b) one or more fragrance ingredients, where the constituents of (a) and (b) form a fragrance component, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; Vertofix Coeur; or combinations thereof. Other embodiments may be directed to compositions comprising 1,3-BG (such as but not limited to, bioBG) and one or more fragrance ingredients that express similar odors or scents to those presented here, or when in combination with other fragrance ingredients express similar odors, scents, or fragrance profiles.

Another embodiment of the disclosure provides a composition comprising one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.; or combinations thereof.

In some embodiments, the composition of the disclosure comprises one or more fragrance ingredients selected from the group consisting of: Aldehyde C-12 Lauric; Aldehyde C14 (Undecalactone γ); Benzyl Acetate; Citral; Cyclal C/Triplal; Damascenone; Dihydro Myrcenol; Dimetol; Ethyl Vanillin; Eucalyptol; Fennaldehyde; Heliotropine; Cis-3-Hexenol; Humus Ether; Isoamyl Acetate; Isobornyl Acetate; Methyl Geosmin; Nootkatone; Toscanol; and Vanillin; or combinations thereof.

One embodiment of the disclosure provides such compositions, where the one or more fragrance ingredients is selected from the group consisting of: Aldehyde C-10; Aldehyde C-12 Lauric; Aldehyde C-12 MNA; Aldehyde Mandarine; Allyl Amyl Glycolate; Ambrinol; Ambrox Super (Ambroxan); Anisic Aldehyde; Anisyl Acetate; Bacdanol; Caryophyllene; Cashmeran; Cetalox/Ambrox; Cinnamalva; Dihydro Eugenol; Evernyl; Fructone; Galaxolide; Hedione; Cis-3-Hexenyl Salicylate; Linalool; Precyclemone B; and Vertofix Coeur; or combinations thereof.

In yet a further embodiment, such compositions of the disclosure comprise one or more fragrance ingredients selected from the group consisting of: Aldehyde C-11 Undecylenic; Anethole; Clearwood; Dibenzyl Ether; Ethylene Brassylate; Grisalva; Iso E Super; Linalyl Acetate; Methyl Hexyl Ketone; Muscenone; Paradisamide; and Terpinyl Acetate; or combinations thereof.

A further embodiment of the disclosure provides such compositions comprising one or more fragrance ingredients selected from the group consisting of: Aldehyde C-12 Lauric; Aldehyde C-12 MNA; Aldehyde C14 (Undecalactone γ); Ambrinol; Anisic Aldehyde; Anisyl Acetate; Bacdanol; Benzyl Acetate; Cashmeran; Cinnamalva; Citral; Cyclal C/Triplal; Dihydro Eugenol; Dihydro Myrcenol; Dimetol; Ethyl Vanillin; Eucalyptol; Evernyl; Fructone; Hedione; Heliotropine; Cis-3-Hexenol; Humus Ether; Isoamyl Acetate; Linalool; Nootkatone; Paradisamide; and Vanillin; or combinations thereof.

In yet another embodiment, the composition of the disclosure comprises one or more fragrance ingredients selected from the group consisting of: Aldehyde C-10; Aldehyde C-11 Undecylenic; Allyl Amyl Glycolate; Linalyl Acetate; and Terpinyl Acetate; or combinations thereof.

Some embodiments of the disclosure provide a composition comprising one or more fragrance ingredients and 1,3-BG (e.g., bioderived 1,3-BG), where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Anethole; Anisic Aldehyde; Anisyl Acetate; Bacdanol; Benzyl Acetate; Cashmeran; Cetalox/Ambrox; Cinnamalva; Citral; Clearwood; Cyclal C/Triplal; Damascenone; Dihydro Eugenol; Dihydro Myrcenol; Dimetol; Ethyl Vanillin; Eucalyptol; Evernyl; Fennaldehyde; Fructone; Grisalva; Hedione; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur; or combinations thereof.

In one embodiment, the composition of the disclosure comprises one or more fragrance ingredients and 1,3-BG, such as but not limited to, bioBG, where the one or more fragrance ingredients is selected from the group consisting of: Ambrox Super (Ambroxan); Dibenzyl Ether; Methyl Ionone Gamma; Ethylene Brassylate; and Galaxolide; Caryophyllene; or combinations thereof.

Another embodiment of the disclosure provides a composition comprising one or more fragrance ingredients and 1,3-BG, e.g., bioderived 1,3-BG, where the one or more fragrance ingredients is selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Grisalva; Galaxolide; Heliotropine; Cis-3-Hexenol; Humus Ether; Isoamyl Acetate; Linalool; Linalyl Acetate; Nootkatone; Paradisamide; Terpinyl Acetate; and Vanillin; or combinations thereof.

In some embodiments, the composition of the disclosure comprises one or more fragrance ingredients and 1,3-BG, such as but not limited to, bioderived 1,3-BG, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur; or combinations thereof.

A further embodiment of the disclosure provides a composition comprising one or more fragrance ingredients and 1,3-BG (e.g., bioderived 1,3-BG), where the one or more fragrance ingredients is selected from the group consisting of: Aldehyde C14 (Undecalactone γ); Ambrox Super (Ambroxan); Anethole; Anisic Aldehyde; Anisyl Acetate; Bacdanol; Benzyl Acetate; Clearwood; Evernyl; Fructone; Hedione; Cis-3-Hexenyl Salicylate; Iso E Super; Methyl Ionone Gamma; Muscenone; and Vertofix Coeur; or combinations thereof.

In yet another embodiment, the composition of the disclosure comprises one or more fragrance ingredients and 1,3-BG, such as, but not limited to bioderived 1,3-BG, where the fragrance ingredient is selected from: Anethole; Anisic Aldehyde; Anisyl Acetate; Benzyl Acetate; Cetalox/Ambrox; Dibenzyl Ether; Evernyl; Hedione; Cis-3-Hexenyl Tiglate; Lime Oxide; Precyclemone B; or combinations thereof.

Some embodiments of the disclosure provide a composition comprising one or more fragrance ingredients and 1,3-BG, including but not limited to, bioderived 1,3-BG, where the comprises one or more fragrance ingredients is selected from the group consisting of: Aldehyde C-12 Lauric; Aldehyde C-12 MNA; Aldehyde C14 (Undecalactone γ); Allyl Amyl Glycolate; Ambrinol; Anisic Aldehyde; Anisyl Acetate; Cashmeran; Cinnamalva; Citral; Dihydro Eugenol; Dimetol; Eucalyptol; Heliotropine; Cis-3-Hexenol; Humus Ether; Linalool; Nootkatone; and Vanillin; or combinations thereof.

In one embodiment, the composition of the disclosure comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Allyl Amyl Glycolate; Ambrinol; Ambrox Super (Ambroxan); Anethole; Anisic Aldehyde; Anisyl Acetate; Bacdanol; Benzyl Acetate; Cashmeran; Cinnamalva; Citral; Clearwood; Cyclal C/Triplal; Dihydro Eugenol; Dihydro Myrcenol; Dimetol; Ethyl Vanillin; Ethylene Brassylate; Eucalyptol; Evernyl; Galaxolide; Grisalva; Hedione; Heliotropine; Cis-3-Hexenol; Humus Ether; Iso E Super; Isoamyl Acetate; Linalool; Linalyl Acetate; Methyl Ionone Gamma; Muscenone; Nootkatone; Paradisamide; Terpinyl Acetate; Vanillin; and Vertofix Coeur; or combinations thereof.

A further embodiment of the disclosure is directed to such compositions comprising one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Cis-3-Hexenyl Tiglate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Lime Oxide; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix CoeurEvernyl; or combinations thereof.

In yet a further embodiment, such compositions of the disclosure comprise one or more fragrance ingredients selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Galaxolide; Grisalva; Heliotropine; Cis-3-Hexenol; Humus Ether; Isoamyl Acetate; Linalool; Linalyl Acetate; Nootkatone; Paradisamide; Terpinyl Acetate; and Vanillin; or combinations thereof.

One embodiment of the disclosure provides compositions comprising one or more fragrance ingredients and 1,3-BG, for example, bioBG, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur; or combinations thereof.

In some embodiments, such compositions of the disclosure comprise one or more fragrance ingredients selected from the group consisting of: Aldehyde C14 (Undecalactone γ); Citral; Dimetol; Nootkatone; Ambrinol; Aldehyde C-12 Lauric; and Aldehyde C-12 MNA; or combinations thereof.

A further embodiment of the disclosure is directed to a composition of the disclosure comprising one or more fragrance ingredients and 1,3-BG, such as, but not limited to, bioBG, where the one or more fragrance ingredients is not selected from the group consisting of: Ambrox Super (Ambroxan); Anethole; Benzyl Acetate; Caryophyllene; Clearwood; Dibenzyl Ether; Ethylene Brassylate; Evernyl; Fructone; Galaxolide; Grisalva; Hedione; Cis-3-Hexenyl Salicylate; Iso E Super; Linalyl Acetate; Methyl Ionone Gamma; Muscenone; Paradisamide; Terpinyl Acetate; and Vertofix Coeur; or combinations thereof.

In an embodiment, such compositions may comprise one or more fragrance ingredients not selected from the group consisting of: Ambrox Super (Ambroxan); Anethole; Caryophyllene; Dibenzyl Ether; Galaxolide; Grisalva; Cis-3-Hexenyl Salicylate; Iso E Super; Methyl Ionone Gamma; and Vertofix Coeur; or combinations thereof.

One embodiment provides such compositions, where the one or more fragrance ingredients, wherein the fragrance ingredient is not selected from the group consisting of: Ambrox Super (Ambroxan); Anethole; Caryophyllene; Clearwood; Dibenzyl Ether; Ethylene Brassylate; Galaxolide; Grisalva; Cis-3-Hexenyl Salicylate; Iso E Super; Methyl Ionone Gamma; Muscenone; and Vertofix Coeur.

Yet a further embodiment is directed to such compositions of the disclosure where the one or more fragrance ingredients is not selected from the group consisting of: Aldehyde Mandarine; Ambrofix; Cashmeran; Cetalox/Ambrox; Damascenone; Dihydro Ionone Beta; Ethyl Amyl Ketone; Fennaldehyde; Fraistone; Cis-3-Hexenyl Tiglate; Isobornyl Acetate; Lime Oxide; Methyl Cinnamate; Methyl Diantilis; Methyl Geosmin; Methyl Hexyl Ketone; Olibanol; Precyclemone B; and Toscanol.

Another embodiment of the disclosure provides a composition comprising one or more fragrance ingredients and 1,3-BG or, for example, bioderived 1,3-BG, where the one or more fragrance ingredient is not selected from: Caryophyllene; Dibenzyl Ether; or combinations thereof. In some embodiments, such compositions of the disclosure comprises one or more fragrance ingredients, where the one or more fragrance ingredients is not selected from the group consisting of: Ambrofix; Fraistone; Cis-3-Hexenyl Tiglate; Lime Oxide; Methyl Ionone Gamma; and Olibanol; or combinations thereof.

Other embodiments provide such compositions of the disclosure, where the one or more fragrance ingredients is not Methyl Ionone Gamma or Olibanol; or combinations thereof.

In another embodiment, such compositions of the disclosure comprise one or more fragrance ingredients, wherein the one or more fragrance ingredients is not selected from the group consisting of: Ambrofix; Fraistone; Cis-3-Hexenyl Tiglate; Lime Oxide; and Olibanol; or combinations thereof.

In one embodiment, such compositions of the disclosure comprise one or more fragrance ingredients, where the one or more fragrance ingredients is not selected from the group consisting of: Ethyl Amyl Ketone; Cashmeran; Dihydro Ionone Beta; Methyl Cinnamate; and Methyl Diantilis; or combinations thereof.

Some embodiments of the disclosure are directed to a composition comprising one or more fragrance ingredients and 1,3-BG or bioBG, where the one or more fragrance ingredients is not selected from the group consisting of: Caryophyllene; Dibenzyl Ether; Fructone; and Cis-3-Hexenyl Salicylate; or combinations thereof.

In one embodiment, fragrance ingredients may be isolated or extracted from natural products, including, but not limited to, flowers, fruits, vegetables, plants, trees, and parts or portions thereof. Isolation of fragrance ingredients in the form of, for example, essential oils, absolutes, concretes, and carbon dioxide extracts, may be accomplished by any one of the commonly known and used methods, including but not limited to, distillation, solvent extraction, expression, and enfleurage (e.g., steam distillation, hydrodistillation, cohobation, molecular distillation, cold pressed extraction, and sieving). (De Silva, Tuley. “A manual on the essential oil industry/editor, K. Tuley De Silva.” UNIDO Workshop on Essential Oil and Aroma Chemical Industries (3rd: 1995: Anadolu Universitesi) TBAM-ICS/UNIDO Training Course on Quality Improvement of Essential Oils: 1999: Eskis, ehir, Turkey. United Nations Industrial Development Organization.)

Another embodiment provides for fragrance ingredients obtained by methods of separating components based on differences in evaporation rate of volatile constituents through temperature, pressure, and subsequent condensation taking place within a still is known as distillation. The essential oils found in plants are insoluble in—and have a higher boiling point than—water, allowing the essential oil to vaporize at a lower temperature than it normally would on its own. For example, natural products (e.g., plant, tree, flower) release essential oils and may be collected and/or concentrated under high-pressure steam distillation conditions, hydro- or water/steam distillation, or a combination thereof.

In a further embodiment, the fragrance ingredients of the disclosure may be synthetic or man-made. Since essential oils and other natural ingredients are limited, expensive, or labor-intensive to obtain, synthetic materials that replicate the natural scents may be incorporated into compositions or fragrance compositions to replace and/or reduce the amount of natural ingredients that may be necessary to achieve the desired scent. Synthetic fragrance ingredients may be chemically created, oftentimes from derived from petroleum or petrochemicals, and may be further modified, for example, by structure. One embodiment may be directed to compositions of the disclosure where the one or more fragrance ingredients is selected from synthetic fragrance ingredients, which includes but is not limited to, chemically created or synthesized fragrance ingredients derived from petroleum, petrochemicals; chemically modified natural raw materials; synthesized or chemically modified ingredients identical to natural raw materials, and synthesized or chemically created or modified chemicals that do not exist in nature. Some embodiments provide “fragrance ingredients,” comprising any scented material, either derived from natural products or synthetic, including but not limited to, acetates, alcohols, aldehydes, esters, ethers, ketones, nitriles, nitrogenous or sulfurous heterocyclic compounds, terpene hydrocarbons, and essential oils. (see, e.g., Arctander, Steffen. Perfume & Flavor Chemicals (Aroma Chemicals) Vols. I-III. Lulu.com, 2019; “Journal of Essential Oil Research,” “Perfume and Flavourist.” A fragrance composition comprising 1,3-BG, including but not limited to, a bioBG, and one or more fragrance ingredients may be formulated, using known and commonly used techniques and ingredients, into various types of fragrance compositions as disclosed here, such as but not limited to, perfumes, colognes, aftershave lotions, aerosol sprays, bath oils, soaps, tales, face powders, deodorants, cleaning materials, room deodorizers, and the like, or any product containing a fragrance or scent.

Some embodiments of the disclosure provide compositions comprising multifunctional 1,3-butylene glycol (including, for example, bioBG), which may be used as a carrier, solvent, or diluent (used interchangeably here) for solubilizing or diluting additional ingredients, while also functioning as an anti-microbial (or microbial growth inhibitor), preservative, or combinations thereof. In one embodiment, the composition of the disclosure comprises a 1,3-BG, such as but not limited to bioBG, that inhibits microbial growth. Organoleptic evaluations were performed on each of the fragrance ingredients as described in the Examples demonstrating the properties of 1,3-BG. An observation of any change in odor character was identified as having a variation. If there were any muting or subduing on or of odor, then the odor was characterized as suppressed. If there were any increases in delivery of an expected odor, then the odor was identified as enhanced. During organoleptic evaluation of fragrance ingredients of the disclosure, odor performance was considered through all stages of the evaluation for consistency of the odor or scent of each of the fragrance ingredients.

In some embodiments of the disclosure, compositions comprising 1,3-BG and one or more fragrance ingredients, may further include additional ingredients depending on the application or use of the composition. These additional ingredients may include, but are not limited to, any and all diluents or solvents, dispersion media, antibacterial and antifungal agents, isotonic and absorption delaying or prolonging agents, and the like. The physiologically acceptable carrier, solvent, or diluent, used interchangeably here, does not destroy the fragrance activity or release of the disclosed fragrance ingredient and is nontoxic when applied in amounts sufficient to deliver a fragrance or fragrance notes of the one or more fragrance ingredients. Another embodiment is directed to 1,3-BG compositions comprising one or more fragrance ingredients, and further comprising at least one or more diluents (e.g., 1,3-BG; bioBG; dipropylene glycol; benzyl benzoate; isopropyl myristate; 1,3-propylene glycol; glycerin); or combinations thereof. In some embodiments, the one or more diluents may be in a ratio of 1:1; or ratio in a range of 1:9-9:1; or any other ratios therebetween. For example, compositions of the disclosure may comprise a 1:9, 1:4, 3:7; 2:3; 1:1, or the like, ratio of 1,3-BG to BioBG; a 1:9, 1:4, 3:7; 2:3; 1:1, or the like, ratio of glycerin to BioBG. In a further embodiment, the amount of 1,3-BG present in a composition of the disclosure will be more than the amount of another co-diluent.

In one classification system, there are 13 fragrance classifications or olfactory families: Aldehydic; Amber; Anisic; Balsamic; Citrus; Earthy; Floral; Fruity; Green; Herbal; Musk; Spicy; and Woody. Some other classification systems may include: Citrus; Fruity; Floral; Green; Herbaceous; Musk; Oriental; and Woody. While other olfactory families may include: Citrus; Floral; Ferns; Chypre; Woody; Oriental/Amber; and Leather. One or more fragrance ingredients comprising any and all of these fragrance classifications may be included in the compositions or applications described here.

For example, in one embodiment, the compositions of the disclosure comprising 1,3-BG (including but not limited to bioBG) and one or more fragrance ingredients may have one or more fragrance ingredients selected from any one or more of the fragrance classifications identified in TABLE 1.

TABLE 1 describes various characteristics of each of the fragrance classifications that may be used in compositions and methods of the disclosure.

TABLE 1

| CLASSIFICATION | DESCRIPTION |
|---|---|
| Aldehydic | Animal, powdery or slightly woody notes often enhance the floral bouquet. |
| | The top note is a mixture of aldehydes and hesperidia. |
| Amber | Mixture of vanilla and animal scents with flowers and woods, which |
| | imparts fullness, sweetness, and warmth to a compound. |
| Anisic | Sharp, sweet, spicy, and warm scents derived from a licorice odor, e.g., |
| | Star Anise, Holy Basil, Sweet Basil, Sweet Fennel. Compounds associated |
| | with licorice odors include, e.g., anethole; fenchone; and estragole. |
| Balsamic | A sweet fragrance that imbues rich, warm, resinous, and tenacious qualities |
| | to most compounds. A woodiness associated with fresh-cut, well-seasoned |
| | non-coniferous wood, e.g., maple. |
| Citrus | Composed of hesperidic or citrus scents, e.g., bergamot, lemon, orange, |
| | tangerine and grapefruit, to which other orange-tree elements (orange |
| | blossoms, petit grain or neroli oil) have been added. Floral or even chypre |
| | accords are sometimes present as well. These scents are characterized by |
| | their freshness and lightness. |
| Chypre | Composed of a scent profile of blends of aldehydes built upon a citrus |
| | (Bergamot) and mossy base (Oakmoss). |
| Earthy | A scent of freshly turned soil, or also described as “rooty” as displayed in |
| | vetivert oil or patchouli oil. |
| Fern | A scent having a mossy, lavender, and citrus character, evoking the forest, |
| | damp, and earthy qualities. These scents also provide a cool, damp, and |
| | woody profile to a fragrance. |
| Floral | Scents from one or more flowers. |
| Fruity | Scents from fruits, e.g., peach, mirabell plum, and exotic fruit. |
| Green | Scents that evoke snapped leaves, foliage, and/or green vegetal scents. |
| Herbal | Scents from herbs, e.g., rosemary, thyme, mint, tarragon, marjoram, fennel, |
| | basil, sage, anise, artemisia, calamus, angelica, and spikenard (jatamansi). |
| Leather | Scents with a smokiness related to old fashioned leather goods. |
| Musk | Scents originally from glandular secretions from animals, e.g., musk deer, |
| | or other strong similar odors from animals or plants. The primary organic |
| | compound having this characteristic musk odor is muscone, i.e., a |
| | substituted 15-membered ring ketone, which has a methyl substituent at the |
| | 3 position. |
| Oriental | Scents that evoke heavy, sweet, animal blends with woody undertones that |
| | are typically diffusive and tenacious. |
| Spicy | Scents that are pungent or piquant from spices, e.g., cinnamon, pepper, |
| | cloves, coriander, ginger, saffron, tamarind, caraway, pink pepper, and |
| | oregano. Spices may be “hot/short” intense and burning for a short |
| | duration (e.g., cinnamon), or “cold/long” gentle, prolonged aftertaste with |
| | cooling sensation versus burning (e.g., coriander, caraway, cardamom). |
| Woody | Scents associated with the aroma of fresh cut, dry, wood or fibrous roots, |
| | such as those woody scents, typically of sandalwood, vetiver, or cedar. |

Fragrance notes describe scents that can be sensed in different periods of time: top notes; middle notes; and base notes. Top notes are scents that can be detected immediately. Typically, fragrance ingredients that produce these notes comprise of small molecules that evaporate quickly and are the most volatile of all of the notes. These notes are particularly important for perfumes or fragrances because they provide the initial scent that is sensed. Since these are meant to evaporate quickly, typically top notes are comprised of lighter oils.

Middle (or heart) notes appear when top notes begin to dissipate and may be composed of, for example, 40% to 80% of the total scent. They yield the major scent of the perfume together with the base notes and can be selected to cover the base scent that can sometimes be unpleasant initially. These middle notes are usually more mellow and can appear in the scent profile somewhere between two minutes and one hour, and last even longer (e.g., 2-4 hours).

The period when the middle notes dissipate, and the base notes take over is known as the dry-down period. The scents of base notes typically last the longest out of the different fragrance notes, bringing depth and solidity to a perfume. They appear when the middle notes begin to disappear and can last up to 24 hours. Base notes have fragrance ingredient molecules that evaporate very slowly, and their scents are usually rich and strong, comprising, for example, 10%-25% of the total scent.

Synthetic fragrances may be derived or isolated from natural products or manufactured in the laboratory to simulate natural product odors. Some synthetics are superior to the natural product in uniformity, stability, and availability. Also, synthetic fragrance ingredients may be as costly as natural products.

Compositions comprising 1,3-BG (or for example, bioderived 1,3-butylene glycol (bioBG)) and one or more fragrance ingredients selected from the odor classification group consisting of: aldehydic, amber, anisic, balsamic, citrus, earthy, floral, fruity, green, herbal, musk, spicy, and woody. One embodiment provides such compositions, where the one or more fragrance ingredients is selected from the odor classification group consisting of: aldehydic, amber, anisic, balsamic, citrus, earthy, floral, fruity, green, herbal, musk, spicy, and woody.

In some embodiments of the disclosure, the composition comprises 1,3-BG (or, for example, bioBG) and one or more fragrance ingredients selected from the odor classification group consisting of: aldehydic, balsamic, citrus, earthy, floral, fruity, green, and herbal. Other embodiments of the disclosure provide such compositions, where the one or more fragrance ingredients is selected from the odor classification group consisting of: aldehydic, amber, anisic, balsamic, citrus, earthy, floral, fruity, green, herbal, musk, spicy, and woody. In another embodiment, such compositions of the disclosure comprise 1,3-BG, such as bioBG, and one or more fragrance ingredients selected from the odor classification group consisting of: aldehydic, amber, balsamic, citrus, earthy, floral, fruity, green, herbal, musk, spicy, and woody. One embodiment of the disclosure provides such compositions, where the one or more fragrance ingredients is selected from the odor classification group consisting of: balsamic, citrus, earthy, floral, fruity, green, and herbal. A further embodiment provides such compositions of the disclosure, where one or more fragrance ingredients is selected from the odor classification group consisting of: aldehydic, amber, anisic, musk, spicy, and woody.

Objective analyses of solubility, organoleptic rating, and preservative value further characterize the 1,3-BG, such as but not limited to bioBG, of the disclosure. Organoleptic testing assesses the strength of one or more fragrance notes in different concentrations of 1,3-BG, for example, bioBG, as well as other diluents (e.g., DPG, BB, IPM), as a comparison.

In one embodiment, the 1,3-BG or bioderived 1,3-BG used as a fragrance ingredient diluent does not interfere with the biological activity or fragrance notes of the fragrance ingredient. Moreover, since 1,3-BG or bioBG is substantially or essentially non-odorous, the scent from fragrance ingredients are not suppressed and in fact may be enhanced by 1,3-BG or bioBG. In some embodiments, the (R)-1,3-butylene glycol, the (S)-1,3-butylene glycol, or racemic mixtures of the (R)-1,3-butylene glycol and the (S)-1,3-butylene glycol derived from 1,3-BG or bioBG provides characteristics that avoid masking or adversely changing the odor or scent of a fragrance. Other embodiments are directed to the (R)-1,3-butylene glycol that provides this benefit of not suppressing or masking, or adversely changing the scent or notes of a fragrance ingredient or fragrance composition.

Another embodiment is directed to this 1,3-BG, including the natural, sustainable bioBG, diluent, which, when combined with one or more fragrance ingredients, provides a clearer and more uninhibited reveal of fragrance notes in polar fragrance ingredients. Polarity describes the distribution of electrical charge among the atoms joined by chemical bonds. The polarity of a bond results from the relative electronegativities of the elements, where the electronegativity is the power of an atom to attract elements toward itself when part of a compound. An ingredient has the tendency to dissolve more readily in a diluent of similar polarity. Moreover, non-polar ingredient is considered lipophilic, and polar ingredient is hydrophilic.

In one embodiment of the disclosure, the one or more fragrance ingredients in 1,3-BG diluent comprise, for example, polar ingredients that can be present in a composition or fragrance component in an amount of: 50% or more (e.g., 55%, 60%, 65%, 70%, 75%, 80%, 85%, 90%, 95%, 97%, 99%, 100%); less than or equal to 100% (e.g., 99%, 98%, 96%, 94%, 92%, 88%, 86%, 84%, 82%, 78%, 76%, 74%, 72%, 68%, 66%, 64%, 62%, 58%, 56%, 54%, 52%), or in those compositions or fragrance components that comprise more polar fragrance ingredients than non-polar fragrance ingredients. Furthermore, 1,3-BG (e.g., bioBG) provides an improvement of fragrance note strength in polar fragrance ingredients, or more polar than non-polar fragrance ingredients, thereby maintaining the true to character perception of the added fragrance ingredient(s). As some fragrance ingredients may be very expensive, e.g., flower extracts costing more than $35,000 per kilogram, reaching a desired fragrance level with a smaller quantity of a source fragrance ingredient, whether natural or synthetic, can generate significant savings. The 1,3-BG of the disclosure also increases the value of a fragrance ingredient in that a more predictable outcome from formulation to compounding, i.e., when fragrance ingredients are added to the fragrance component of a fragrance composition.

The 1,3-BG, including but not limited to bioBG, of the disclosure, as observed during organoleptic assessment, provides a solubility of moderate to excellent, which is superior to other natural diluents, such as benzyl benzoate and isopropyl myristate, and comparable to petroleum-derived dipropylene glycol. See, e.g., TABLES 3-11.

The term “effective amount” of a fragrance ingredient or diluent, as used herein, is an amount sufficient to effect desired results, such as the preferred scent of a fragrance ingredient or combination of fragrance ingredients, and, as such, an “effective amount” depends upon the context in which it is being applied. In some embodiments, one or more fragrance ingredients in 1,3-BG or, for example, bioderived 1,3-BG, of a fragrance component or fragrance composition is administered in an effective amount for obtaining or maintaining a pleasant scent or odor, or alternatively, avoiding unpleasant or off-putting odors on the body, clothes, room, etc., wherever the fragrance composition or fragrance component is applied or emitted.

As used herein, the term “subject” or “consumer” refers to any organism to which a composition in accordance with the disclosure may be applied or administered, e.g., for experimental, diagnostic, organoleptic, or cosmetic purposes. Typical subjects include any animal (e.g., mammals such as humans, mice, rats, rabbits, and non-human primates, etc.). A subject or consumer in need thereof is typically a subject for whom it is desirable to smell agreeably, exude a pleasant scent, or mask a malodorous aroma as described herein. For example, a subject in need thereof may seek or be in need of treatment, require treatment, be receiving treatment, may be receiving treatment in the future, or a human or animal that is under care by a trained professional for a particular malodorous condition. In another embodiment, a subject or consumer exposed to a malodorous aroma or scent would benefit from exposure to a composition of the disclosure, where the composition comprises a fragrance component comprising a 1,3-BG or bioBG and one or more fragrance ingredients. In some embodiments, such a composition is a household product, e.g., a room freshener, diffuser, which masks a malodorous scent or freshens the room.

The term “fragrance composition” or “composition,” as used herein, represents a composition containing one or more fragrance ingredients described here and formulated with a physiologically acceptable diluent or solvent, used interchangeably here. In a fragrance composition, or composition that has a fragrance, such as a perfume, 20%-40% of the observable liquid may be comprised of a diluent, i.e., the delivery system for scents of the fragrance ingredients. The “log P” value, as used here, indicates whether a substance or fragrance ingredient will be absorbed by humans, animals, plants, or living tissue; diffused or disseminated by water.

One embodiment provides for diluents that are imperceptible or nearly imperceptible to the fragrance (e.g., 1,3-BG; bioBG). Diluents may also be used as a metering tool in order to modify by increasing or decreasing the strength of an odor or notes in a fragrance ingredient, thereby providing precise control and clarity of the olfactory notes of the fragrance ingredients. Another embodiment may be directed to the use of one or more diluents depending on the desired fragrance ingredient(s) and solubility.

In yet another embodiment of the disclosure, the one or more fragrance ingredients is selected from the characteristics consisting of: a molecular weight greater than or equal to 90 g/mol (e.g., 100 g/mol; 120 g/mol; 125 g/mol; 130 g/mol; 140 g/mol; 150 g/mol; 175 g/mol; 185 g/mol; 200 g/mol; 220 g/mol; 250 g/mol; 270 g/mol), less than or equal to 270 g/mol (e.g., 260 g/mol; 250 g/mol; 240 g/mol; 230 g/mol; 220 g/mol; 210 g/mol; 200 g/mol; 185 g/mol; 175 g/mol; 165 g/mol; 155 g/mol; 145 g/mol; 135 g/mol; 125 g/mol; 120 g/mol; 100 g/mol; 95 g/mol), or in a range of 90 g/mol to 270 g/mol (e.g., 100 g/mol-270 g/mol; 130 g/mol-260 g/mol; 145 g/mol-250 g/mol; 150 g/mol-220 g/mol; 175 g/mol-200 g/mol); a log P value greater than or equal to 0.5 (e.g., 1, 2, 3, 4, 5, 6, 7), less than or equal to 7 (e.g., 6.5, 5.5, 4.5, 3.5, 2.5, 1.5, 0.5), or in a range of 0.5 to 7 (e.g., 0.9-7; 1-6.7; 1.5-6.5; 2-6.3; 2.5-6; 2.7-5.5; 3-5; 3.5-4.5; 3.7-4.3); a mixture of polar and non-polar fragrance ingredients; a mixture of more polar fragrance ingredients than non-polar fragrance ingredients; essentially polar fragrance ingredients; a mixture of natural and synthetic fragrance ingredients; essentially synthetic fragrance ingredients; and combinations or mixtures of any of the aforementioned characteristics.

A further embodiment may be directed to a composition of the disclosure comprising 1,3-BG (including but not limited to, bioBG, (R)-1,3-BG, mixtures thereof) and one or more fragrance ingredients comprising a solubility characteristic of moderate (2); good (3); excellent (4); or combinations thereof. In one embodiment, the 1,3-BG composition of the disclosure further comprises one or more fragrance ingredients not selected from those ingredients comprising a poor solubility characteristic (1).

The composition in a further embodiment of the disclosure is directed to a fragrance composition or fragrance component comprising 1,3-BG and one or more fragrance ingredients present in an amount greater than or equal to 0.01% by weight (e.g., 0.06%; 0.11%; 0.16%; 0.21%; 0.26%; 0.31%; 0.36%; 0.41%; 0.46%; 0.51%; 0.56%; 0.61%; 0.66%; 0.71%; 0.76%; 0.81%; 0.86%; 0.91%; 0.96%; 1%; 1.05%; 1.25%; 1.5%; 1.75%; 2%; 2.5%; 3%; 3.5%; 4%; 4.5%; 5%; 5.5%; 6%; 6.5%; 7%; 7.5%; 8%; 8.5%; 9%; 9.5%; 10%; 10.5%; 15%; 20%; 25%; 30%; 35%; 40%; 45%; 50%; 55%; 60%) relative to the total % by weight of fragrance component or composition. Another aspect is directed to one or more fragrance ingredients present in an amount less than or equal to 60% by weight relative (e.g., 55%; 50%; 45%; 40%; 35%; 30%; 25%; 20%; 15%; 10.5%; 10%; 9.5%; 9%; 8.5%; 8%; 7.5%; 7%; 6.5%; 6%; 5.5%; 5%; 4.5%; 4%; 3.5%; 3%; 2.5%; 2%; 1.75%; 1.5%; 1.25%; 1.05%; 1%; 0.95%; 0.9%; 0.85%; 0.8%; 0.75%; 0.7%; 0.65%; 0.6%; 0.55%; 0.5%; 0.45%; 0.4%; 0.35%; 0.3%; 0.25%; 0.2%; 0.15%; 0.1%; 0.05%; 0.01%) to the total % by weight of fragrance component or composition. In yet a further aspect, the one or more fragrance ingredients is present in an amount of 0.015% by weight to 60% by weight (e.g., 0.05%-55%; 0.1%-45%; 0.5%-30%; 1%-25%; 2%-20%; 5%-15%; 7%-10%) relative to the total % by weight of fragrance component or composition.

In one embodiment, the disclosure provides a composition comprising: (a) 1,3-BG, for example, a bioderived 1,3-butylene glycol (bioBG); and (b) one or more fragrance ingredients, where the components of (a) and (b) form a fragrance component, where the composition or fragrance component is selected from the group consisting of: non-irritating, non-sensitizing, non-allergenic; and non-greasy.

Another embodiment of the disclosure is directed to a method, comprising applying to skin of a subject, a composition comprising (a) 1,3-BG, such as but not limited to a bioderived 1,3-butylene glycol (bioBG); and (b) one or more fragrance ingredients, where the components of (a) and (b) form a fragrance component, where the composition is a fragrance composition. A further aspect of the fragrance composition provides one or more fragrance ingredients selected from the group of characteristics consisting of: polar, synthetic, excellent solubility, clear clarity, and moderate to bright headspace.

It will be understood that the compositions of the disclosure include cosmetic and personal care compositions, such as fragrance compositions, as well as household care products, such as room fresheners or deodorizers, and the like, or any product containing a fragrance, unless otherwise specified. In some embodiments, the composition is manufactured or sold with the approval of a governmental regulatory agency as part of a safety for use in a mammal. Compositions can be formulated, for example, for topical administration (e.g., as an extract or parfum, eau de parfum, eau de toilette, eau de cologne, eau de fraiche, aftershave, cream, gel, lotion, ointment, oil (e.g., essential oil, massage oil, aromatherapy), shampoo, hair conditioner, shower gel, soap, deodorant, anti-perspirant) or in any other formulation that utilizes a fragrance (e.g., air freshener, room and/or carpet deodorizer/freshener, laundry detergent, fabric softener, dryer sheet). The bioBG of the disclosure is a multifunctional ingredient, which may be used in a variety of personal care products and cosmetics, including serums, body scrubs, masks, cleansers, and the like, in combination with at least one fragrance ingredient, or one or more fragrance ingredients, to result in a desired scent or combination of scents.

As used herein, the phrase “physiologically acceptable” means generally safe for contact with biologic tissues, particularly at the levels employed. Physiologically acceptable is used interchangeably with physiologically compatible. The compositions of the disclosure comprise physiologically acceptable or compatible components, including but not limited to, fragrance ingredients and diluents (e.g., bioBG, DPG, BB, IPM) or additional solvents or diluents, used interchangeably.

Another embodiment of the disclosure provides a method, comprising applying a composition of the disclosure to skin of a subject, where the composition is a fragrance composition containing any one or more of the fragrance ingredients disclosed here. In one embodiment, the one or more fragrance ingredients is selected from a group of characteristics: polar, synthetic, excellent solubility, clear clarity, and moderate to bright headspace.

A further embodiment provides a use of 1,3-BG, such as but not limited to, a bioderived 1,3-butylene glycol (bioBG), as a diluent in a fragrance component, wherein the fragrance component further comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur; or combinations thereof.

Another embodiment provides for uses of 1,3-BG in such compositions, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur; or combinations thereof.

Yet an additional embodiment provides uses of 1,3-BG in compositions of the disclosure, where such compositions of the disclosure provide one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.

In an embodiment of the disclosure, the use of 1,3-BG, including but not limited to, bioBG, as a diluent may comprise a composition having one or more fragrance ingredients selected from the group consisting of: Aldehyde C-12 Lauric; Aldehyde C14 (Undecalactone γ); Benzyl Acetate; Citral; Cyclal C/Triplal; Damascenone; Dihydro Myrcenol; Dimetol; Ethyl Vanillin; Eucalyptol; Fennaldehyde; Heliotropine; Cis-3-Hexenol; Humus Ether; Isoamyl Acetate; Isobornyl Acetate; Methyl Geosmin; Nootkatone; Toscanol; and Vanillin.

Another embodiment is directed to the use of 1,3-BG in a composition, where the compositions of the disclosure comprise one or more fragrance ingredients selected from the group consisting of: Aldehyde C-10; Aldehyde C-12 Lauric; Aldehyde C-12 MNA; Aldehyde Mandarine; Allyl Amyl Glycolate; Ambrinol; Ambrox Super (Ambroxan); Anisic Aldehyde; Anisyl Acetate; Bacdanol; Caryophyllene; Cashmeran; Cetalox/Ambrox; Cinnamalva; Dihydro Eugenol; Evernyl; Fructone; Galaxolide; Hedione; Cis-3-Hexenyl Salicylate; Linalool; Precyclemone B; and Vertofix Coeur. In a further embodiment, the use of 1,3-BG (e.g., bioBG) as a diluent may comprise such compositions of the disclosure, where the one or more fragrance ingredients is selected from the group consisting of: Aldehyde C-11 Undecylenic; Anethole; Clearwood; Dibenzyl Ether; Ethylene Brassylate; Grisalva; Iso E Super; Linalyl Acetate; Methyl Hexyl Ketone; Muscenone; Paradisamide; and Terpinyl Acetate.

Yet another embodiment provides for use of 1,3-BG, including bioBG, as a diluent of, for example, one or more fragrance ingredients selected from the group consisting of: Aldehyde C-10; Aldehyde C-11 Undecylenic; Allyl Amyl Glycolate; Linalyl Acetate; and Terpinyl Acetate; or combinations thereof.

Yet another use of 1,3-BG or, for example, bioBG provides a composition of the disclosure that comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Anethole; Anisic Aldehyde; Anisyl Acetate; Bacdanol; Benzyl Acetate; Cashmeran; Cetalox/Ambrox; Cinnamalva; Citral; Clearwood; Cyclal C/Triplal; Damascenone; Dihydro Eugenol; Dihydro Myrcenol; Dimetol; Ethyl Vanillin; Eucalyptol; Evernyl; Fennaldehyde; Fructone; Grisalva; Hedione; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur, or combinations thereof.

One use of 1,3-BG or bioBG may occur in a composition of the disclosure, where the one or more fragrance ingredients selected from the group consisting of: Ambrox Super (Ambroxan); Caryophyllene; Dibenzyl Ether; Methyl Ionone Gamma; Ethylene Brassylate; and Galaxolide; or combinations thereof.

In a further use, the composition comprising one or more fragrance ingredients selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Grisalva; Galaxolide; Heliotropine; Cis-3-Hexenol; Humus Ether; Isoamyl Acetate; Linalool; Linalyl Acetate; Nootkatone; Paradisamide; Terpinyl Acetate; and Vanillin; or combinations thereof.

Yet another use is directed to a composition of the disclosure that comprises comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur.

In one embodiment, the use comprises a composition of the disclosure, where one or more fragrance ingredients is selected from the group consisting of: Aldehyde C14 (Undecalactone γ); Ambrox Super (Ambroxan); Anethole; Anisic Aldehyde; Anisyl Acetate; Bacdanol; Benzyl Acetate; Clearwood; Evernyl; Fructone; Hedione; Cis-3-Hexenyl Salicylate; Iso E Super; Methyl Ionone Gamma; Muscenone; and Vertofix Coeur; or combinations thereof.

A further use of 1,3-BG, including but not limited to bioBG, as a diluent is provided, where the composition comprises one or more fragrance ingredients selected from the group consisting of: Anethole; Anisic Aldehyde; Anisyl Acetate; Benzyl Acetate; Cetalox/Ambrox; Dibenzyl Ether; Evernyl; Hedione; Cis-3-Hexenyl Tiglate; Lime Oxide; and Precyclemone B; or combinations thereof.

In another use of the disclosure, 1,3-BG, such as bioBG, may be used as a diluent in a composition further comprising one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Cis-3-Hexenyl Tiglate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Fennaldehyde; Galaxolide; Grisalva; Hedione; Heliotropine; Cis-3-Hexenol; Cis-3-Hexenyl Salicylate; Humus Ether; Iso E Super; Isoamyl Acetate; Isobornyl Acetate; Lime Oxide; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur; Isobornyl Acetate; Methyl Geosmin; Toscanol; Aldehyde Mandarine; Methyl Hexyl Ketone; Ambrofix; Anethole; Anisic Aldehyde; Anisyl Acetate; Benzyl Acetate; Dibenzyl Ether; Evernyl; Cetalox/Ambrox; Evernyl; Hedione; Lime Oxide; Precyclemone B; and Cis-3-Hexenyl Tiglate; or combinations thereof. One embodiment of the disclosure provides the use of 1,3-BG, such as bioBG, in a composition of the disclosure, where the composition further comprises one or more fragrance ingredients is selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Galaxolide; Grisalva; Heliotropine; Cis-3-Hexenol; Humus Ether; Isoamyl Acetate; Linalool; Linalyl Acetate; Nootkatone; Paradisamide; Terpinyl Acetate; and Vanillin; or combinations thereof.

In another embodiment, uses of 1,3-BG (e.g., bioBG) are provided in such compositions, where the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur; or combinations thereof.

In yet another use embodiment, the composition comprises one or more fragrance ingredients selected from the group consisting of: Aldehyde C-12 Lauric; Aldehyde C-12 MNA; Aldehyde C14 (Undecalactone γ); Allyl Amyl Glycolate; Ambrinol; Anisic Aldehyde; Anisyl Acetate; Cashmeran; Cinnamalva; Citral; Dihydro Eugenol; Dimetol; Eucalyptol; Heliotropine; Cis-3-Hexenol; Humus Ether; Linalool; Nootkatone; and Vanillin; or combinations thereof.

A further embodiment provides for the use, where the composition comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Allyl Amyl Glycolate; Ambrinol; Ambrox Super (Ambroxan); Anethole; Anisic Aldehyde; Anisyl Acetate; Bacdanol; Benzyl Acetate; Cashmeran; Cinnamalva; Citral; Clearwood; Cyclal C/Triplal; Dihydro Eugenol; Dihydro Myrcenol; Dimetol; Ethyl Vanillin; Ethylene Brassylate; Eucalyptol; Evernyl; Galaxolide; Grisalva; Hedione; Heliotropine; Cis-3-Hexenol; Humus Ether; Iso E Super; Isoamyl Acetate; Linalool; Linalyl Acetate; Methyl Ionone Gamma; Muscenone; Nootkatone; Paradisamide; Terpinyl Acetate; Vanillin; and Vertofix Coeur; or combinations thereof.

In one embodiment, the use of 1,3-BG or, for example, bioBG as a diluent occurs where the composition comprises one or more fragrance ingredients selected from the group consisting of: Aldehyde C14 (Undecalactone γ); Citral; Dimetol; Nootkatone; Ambrinol; Aldehyde C-12 Lauric; and Aldehyde C-12 MNA; or combinations thereof.

Another embodiment provides for the use of 1,3-BG, such as, but not limited to, bioBG as a diluent where the composition of the disclosure comprises one or more fragrance ingredients, wherein the fragrance ingredient is not selected from the group consisting of: Ambrox Super (Ambroxan); Anethole; Benzyl Acetate; Caryophyllene; Clearwood; Dibenzyl Ether; Ethylene Brassylate; Evernyl; Fructone; Galaxolide; Grisalva; Hedione; Cis-3-Hexenyl Salicylate; Iso E Super; Linalyl Acetate; Methyl Ionone Gamma; Muscenone; Paradisamide; Terpinyl Acetate; and Vertofix Coeur; or combinations thereof.

A further embodiment is directed to the use of 1,3-BG (e.g., bioBG) as a diluent, where the composition comprises one or more fragrance ingredients, wherein the fragrance ingredient is not selected from the group consisting of: Ambrox Super (Ambroxan); Anethole; Caryophyllene; Dibenzyl Ether; Galaxolide; Grisalva; Cis-3-Hexenyl Salicylate; Iso E Super; Methyl Ionone Gamma; and Vertofix Coeur; or combinations thereof.

One embodiment provides for the use of 1,3-BG or, for example, bioBG as a diluent in a composition, where the composition comprises one or more fragrance ingredients, wherein the fragrance ingredient is not selected from the group consisting of: Ambrox Super (Ambroxan); Anethole; Caryophyllene; Clearwood; Dibenzyl Ether; Ethylene Brassylate; Galaxolide; Grisalva; Cis-3-Hexenyl Salicylate; Iso E Super; Methyl Ionone Gamma; Muscenone; and Vertofix Coeur; or combinations thereof.

In some embodiments, the use of 1,3-BG as a diluent, provides for a composition comprising one or more fragrance ingredients, wherein the fragrance ingredient is not selected from the group consisting of: Aldehyde Mandarine; Ambrofix; Cashmeran; Cetalox/Ambrox; Damascenone; Dihydro Ionone Beta; Ethyl Amyl Ketone; Fennaldehyde; Fraistone; Cis-3-Hexenyl Tiglate; Isobornyl Acetate; Lime Oxide; Methyl Cinnamate; Methyl Diantilis; Methyl Geosmin; Methyl Hexyl Ketone; Olibanol; Precyclemone B; and Toscanol; or combinations thereof.

Other embodiments are directed to the use of 1,3-BG (e.g., bioBG) as a diluent in a composition, where the composition comprises one or more fragrance ingredients, where the one or more fragrance ingredients is not selected from the group consisting of: Caryophyllene; and Dibenzyl Ether; or combinations thereof.

In a further embodiment, use of 1,3-BG as a diluent in compositions of the disclosure, where the composition comprises one or more fragrance ingredients, where the one or more fragrance ingredients is not selected from the group consisting of: Ambrofix; Fraistone; Cis-3-Hexenyl Tiglate; Lime Oxide; Methyl Ionone Gamma; and Olibanol; or combinations thereof.

Some embodiments provide for use of 1,3-BG, such as but not limited to, bioBG in compositions of the disclosure, where the composition comprises one or more fragrance ingredients, and the one or more fragrance ingredients is not selected from the group consisting of: Methyl Ionone Gamma or Olibanol; or combinations thereof.

In one embodiment, use of 1,3-BG as a diluent in compositions of the disclosure may be provided where the composition comprises one or more fragrance ingredients, wherein the fragrance ingredient is not selected from the group consisting of: Ambrofix; Fraistone; Cis-3-Hexenyl Tiglate; Lime Oxide; and Olibanol; or combinations thereof.

Another use of the disclosure provides for such compositions comprising one or more fragrance ingredients, where the one or more fragrance ingredients is not selected from the group consisting of: Ethyl Amyl Ketone; Cashmeran; Dihydro Ionone Beta; Methyl Cinnamate; and Methyl Diantilis; or combinations thereof.

A further use of 1,3-BG (e.g., bioBG) as a diluent is directed to compositions comprising one or more fragrance ingredients, where the one or more fragrance ingredients is not selected from the group consisting of: Caryophyllene; Dibenzyl Ether; Fructone; and Cis-3-Hexenyl Salicylate; or combinations thereof.

Another embodiment provides for the use of 1,3-BG, including but not limited to, 1,3-BG, bioBG, or a mixture of 1,3-BG and bioBG, as a diluent in a fragrance composition, where the fragrance composition further comprises one or more fragrance ingredients, and the 1,3-BG is selected from the group consisting of: (R)-1,3-BG; (S)-1,3-BG; and a mixture of: (R)-1,3-BG and (S)-1,3-BG. In one embodiment, the 1,3-BG is (R)-1,3-BG; or (S)-1,3-BG; or a mixture of: (R)-1,3-BG and (S)-1,3-BG. Some embodiments provide for a mixture that comprises 75% or greater of (R)-1,3-BG (e.g., 80%, 85%, 90%, 95%, 97%, 99%, 99.5%, 100%); or the mixture comprises 25% or less of (S)-1,3-BG (e.g., 20%, 15%, 10%, 5%, 4%, 3%, 2%, 1%, 0.5%, 0.1%, 0.05%, 0.001%; 0%). One embodiment provides the use of 1,3-BG as a diluent, where the 1,3-BG is a bioBG.

In a further embodiment, another use of 1,3-BG or, for example, bioBG, can be as amicrobial growth inhibitor or preservative.

Another embodiment is directed to the use of 1,3-BG (e.g., bioBG) as a diluent for one or more fragrance ingredients, where the one or more fragrance ingredients each has a molecular weight of or greater than 90 g/mol (e.g., 100 g/mol; 120 g/mol; 125 g/mol; 130 g/mol; 140 g/mol; 150 g/mol; 175 g/mol; 185 g/mol; 200 g/mol; 220 g/mol; 250 g/mol; 270 g/mol); a molecular weight of or less than 270 g/mol (e.g., 260 g/mol; 250 g/mol; 240 g/mol; 230 g/mol; 220 g/mol; 210 g/mol; 200 g/mol; 185 g/mol; 175 g/mol; 165 g/mol; 155 g/mol; 145 g/mol; 135 g/mol; 125 g/mol; 120 g/mol; 100 g/mol; 95 g/mol); or a molecular weight of 90 g/mol to 270 g/mol (e.g., 100 g/mol-270 g/mol; 130 g/mol-260 g/mol; 145 g/mol-250 g/mol; 150 g/mol-220 g/mol; 175 g/mol-200 g/mol).

In one embodiment, the use of 1,3-BG or, for example, bioBG as a diluent in a fragrance composition provides one or more fragrance ingredients each having a log P value of or greater than 0.5 (e.g., 1, 2, 3, 4, 5, 6, 7); a log P value of or less than 7 (e.g., 6.5, 5.5, 4.5, 3.5, 2.5, 1.5, 0.5); or a log P value of 0.5 to 7 (e.g., 0.9-7; 1-6.7; 1.5-6.5; 2-6.3; 2.5-6; 3-5; 3.5-4.5; 3.7-4.3).

A further embodiment may be directed to use of a 1,3-BG as a diluent in a composition of the disclosure comprising 1,3-BG (including but not limited to, bioBG, (R)-1,3-BG, mixtures thereof) and one or more fragrance ingredients comprising a solubility characteristic of moderate (2); good (3); excellent (4); or combinations thereof. In one embodiment, the 1,3-BG composition of the disclosure further comprises one or more fragrance ingredients not selected from those ingredients comprising a poor solubility characteristic (1).

Another embodiment of the disclosure provides a use of 1,3-BG in a composition, where the composition comprising one or more fragrance ingredients comprises a mixture of polar and non-polar fragrance ingredients; or comprises a mixture comprising more polar fragrance ingredients than non-polar fragrance ingredients; or comprises or consists essentially of polar fragrance ingredients. Some embodiments may be directed to 1,3-BG (including but not limited to: 1,3-BG; bioBG; (R)-1,3-BG; (S)-1,3-BG; mixtures of (R)-1,3-BG and (S)-1,3-BG) fragrance compositions comprising one or more fragrance ingredients, where each of or at least one of the one or more fragrance ingredients is polar. In yet a further embodiment of the disclosure, the use is directed to a fragrance composition comprising one or more fragrance ingredients comprising: a mixture of natural fragrance ingredients and synthetic fragrance ingredients; or a mixture of more synthetic fragrance ingredients than natural fragrance ingredients; or comprising or consisting essentially of synthetic fragrance ingredients.

In one embodiment of the disclosure, the use of 1,3-BG, such as but not limited to, bioBG, as a diluent in a fragrance composition comprising one or more fragrance ingredients disclosed here, further comprises at least one or more diluents (e.g., 1,3-BG; bioBG; dipropylene glycol; benzyl benzoate; isopropyl myristate; 1,3-propylene glycol; glycerin). In some embodiments, one or more diluents may be in a ratio of 1:1; or ratio in a range of 1:9-9:1; or any other ratios therebetween. For example, compositions of the disclosure may comprise a 1:9, 1:4, 3:7; 2:3; 1:1, or the like, ratio of 1,3-BG to BioBG; a 1:9, 1:4, 3:7; 2:3; 1:1, or the like, ratio of glycerin to BioBG. In a further embodiment, the amount of 1,3-BG present in a composition of the disclosure will be more than the amount of another co-diluent.

Another embodiment provides for a use of 1,3-BG as a diluent, where such fragrance compositions of the disclosure comprises a fragrance component comprising one or more fragrance ingredients present in an amount of or greater than 0.01% by weight (e.g., 0.06%; 0.11%; 0.16%; 0.21%; 0.26%; 0.31%; 0.36%; 0.41%; 0.46%; 0.51%; 0.56%; 0.61%; 0.66%; 0.71%; 0.76%; 0.81%; 0.86%; 0.91%; 0.96%; 1%; 1.05%; 1.25%; 1.5%; 1.75%; 2%; 2.5%; 3%; 3.5%; 4%; 4.5%; 5%; 5.5%; 6%; 6.5%; 7%; 7.5%; 8%; 8.5%; 9%; 9.5%; 10%; 10.5%; 15%; 20%; 25%; 30%; 35%; 40%; 45%; 50%; 55%; 60%); one or more fragrance ingredients present in an amount of or less than 60% by weight (e.g., 55%; 50%; 45%; 40%; 35%; 30%; 25%; 20%; 15%; 10.5%; 10%; 9.5%; 9%; 8.5%; 8%; 7.5%; 7%; 6.5%; 6%; 5.5%; 5%; 4.5%; 4%; 3.5%; 3%; 2.5%; 2%; 1.75%; 1.5%; 1.25%; 1.05%; 1%; 0.95%; 0.9%; 0.85%; 0.8%; 0.75%; 0.7%; 0.65%; 0.6%; 0.55%; 0.5%; 0.45%; 0.4%; 0.35%; 0.3%; 0.25%; 0.2%; 0.15%; 0.1%; 0.05%; 0.01%); or one or more fragrance ingredients present in an amount of 0.01% by weight to 60% by weight (e.g., 0.05%-55%; 0.1%-45%; 0.5%-30%; 1%-25%; 2%-20%; 5%-15%; 7%-10%).

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage inclusive of the lowermost and uppermost range, and integrals therebetween, such as for example, 0.1%, 1%, 1.5%, 2.0%, 2.5%, and 3%. Additionally, a range of 0.1 to 3% includes, for example, subsets of the original range including from 0.5% to 2.5%, from 1% to 3%, from 0.1% to 2.5%, etc. It will be understood that the sum of all weight percents (weight %) of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented.

### EXAMPLES

The following examples illustrate specific aspects of the instant description. The examples should not be construed as limiting, as the example merely provides specific understanding and practice of the embodiments and its various aspects.

A master perfumer of MOCA Fragrances (Denville, New Jersey) conducted a study of 105 common fragrance ingredients and four diluents for testing solubility and conducting an organoleptic blotter evaluation. Each ingredient and diluent were evaluated at multiple dilutions, during wet- and dry-down periods, leading to more than 1,200 data points to derive conclusions.

### Example 1: Qualitative Analysis of Bioderived 1,3-Butylene Glycol

A master perfumer conducted a qualitative review of the bioderived 1,3-Butylene Glycol (bioBG) alone, i.e., without any other ingredients such as fragrance ingredients. It was determined that the bioBG was a very pure ingredient both visually and based on the production process known to the master perfumer. The master perfumer also noted a slight green note in the background, beneath the radar.

The master perfumer then qualitatively analyzed the interaction of bioBG and several common fragrance ingredients at various dilutions. The results were the enhancement of the particular scent of the fragrance ingredient, thereby producing a final product that smelled as envisioned. BioBG did not suppress the fragrance of the fragrance ingredients and avoided the use of more of the fragrance ingredient to achieve the desired level of fragrance.

### Example 2: Comparative Blotter Testing

A master perfumer (MOCA Fragrances) compared the bioderived 1,3-butylene glycol to three commonly used diluents in mainstream use: dipropylene glycol, benzyl benzoate, and isopropyl myristate. Their respective solubility, preservative value, and organoleptic rating across multiple dilutions were all evaluated. Organoleptic testing assesses the strength of fragrance expression in different diluents. The results showcased the bioderived 1,3-BG's high performance traits, independent of its attractiveness as a sustainable alternative for fragrances and other formulations.

Specifically, samples of four different dilutions for each fragrance ingredient in each of the different diluents were evaluated on a comparative basis. The different diluents were: bioderived 1,3-butylene glycol (bioBG); dipropylene glycol (DPG); isopropyl myristate (IPM); and benzyl benzoate (BB). Dilutions of 0.01%, 0.1%, 1%, and 10% for each fragrant ingredient in each of the diluents were prepared. Specifically, dilutions of 76 select fragrance ingredients across the odor spectrum were compounded at 10% and 1% and serial diluted to 0.1% and 0.01% for each of the diluents, i.e., bioBG, DPG, IPM, and BB. All ingredients were evaluated at 10% and 1%, 0.1% and 0.01%.

Results were obtained by blotter evaluation of the fragrance ingredients. Blotters were simultaneously dipped in each of the samples of diluted fragrance ingredients in each of the diluents. Odor evaluation was completed by smelling the same dilution of each individual corresponding percentage in comparison to give a value rating to the ingredient on an expert based judgement. The odors across the diluent spectrum were compared and evaluated sequentially from the lowest diluent first to the highest dilution by a master perfumer. Ingredients were first evaluated at the 1% and 10% levels and subsequently upon further dilution at the 0.01% and 0.1% levels one hour later. This allowed for normal diluent level evaluation and strength judgement and later to cross reference the strength at the lower levels validating the evaluation.

Odors of each sample were rated on a 1-10 scale (10 being the highest rating) for each level. Where solubility was not acceptable, the highest odor score could not be higher than 5 (an odor rating was given even in insoluble results). The 1-10 scale applied to each ingredient across the four comparative diluents (i.e., bioderived 1,3-butylene glycol (bioBG); Dipropylene Glycol (DPG); IPM; and benzyl benzoate) at each dilution (i.e., 0.01%, 0.1%, 1%, 10%). For fragrance ingredients having excellent solubility, the organoleptic rating of bioBG was demonstrated to be higher than all other diluents tested, while for moderately soluble fragrance ingredients, bioBG has higher organoleptic ratings than other natural diluents (e.g., BB, IPM) and comparable to dipropylene glycol. BioBG advantageously provides a clear and unobstructed assessment of the fragrance ingredient that is diluted.

Once all data were accumulated and logged, a profile of the performance of bioBG was determined. Graphing the plotting of the data points pointed to a very positive solubility profile with the synthetic ingredients and varying results with the natural essential oils. There appeared to be an affinity for more polar ingredients and lower molecule weight ingredients. Overall, the resulting profile showed promising use in applications with polar environments and requirements in skin application.

The odor comparison rating scale was as follows in TABLE 2.

TABLE 2

| RAT- | |
|---|---|
| ING | DESCRIPTION |
| 10 | Odor has excellent strength in comparison to other diluents at the |
| | same level and demonstrates the expected character of the |
| | ingredient |
| 9 | Odor has very good strength in comparison to the other diluents at |
| | the same level and demonstrates acceptable character of the |
| | ingredient |
| 8 | Odor has good strength in comparison to the other diluents at the |
| | same level and demonstrates acceptable character of the ingredient |
| 7 | Odor has moderate strength in comparison to the other diluents at |
| | the same level and demonstrates acceptable character of the |
| | ingredient |
| 6 | Odor has less strength in comparison to the other diluents at the |
| | same level and demonstrates acceptable character of the ingredient |
| 5 | Odor strength low in comparison to the other diluents at the same |
| | level and demonstrates acceptable character of the ingredient |
| 4 | Odor strength poor in comparison to the other diluents at the same |
| | level and demonstrates acceptable character of the ingredient |
| 3 | Odor strength poor in comparison to the other diluents at the same |
| | level and demonstrates variation in character of the ingredient |
| 2 | Odor strength poor in comparison to the other diluents at the same |
| | level and demonstrates indistinguishable character of the ingredient |
| 1 | Odor low to none and no character of the ingredient |

TABLE 3 shows the average odor value or rating over different concentrations of fragrance ingredients in various diluents (i.e., 0.01%, 0.1%, 1%, 10%) of the blotter comparison analysis. The diluents are: bioderived 1,3-Butylene Glycol (bioBG); Dipropylene Glycol (DPG); Benzyl Benzoate (BB); and Isopropyl Myristate (IPM). The odor value or rating results for each of the individual diluents at the indicated concentrations are in TABLES 4-7.

TABLE 3

| | Bio- | | | |
|---|---|---|---|---|
| | derived | Dipropylene | Benzyl | Isopropyl |
| INGREDIENT | 1,3-BG | Glycol | Benzoate | Myristate |
| Aldehyde C-10 | 9.00 | 10.00 | 7.00 | 8.00 |
| Aldehyde C-11 | 9.00 | 10.00 | 9.00 | 8.00 |
| Undecylenic | | | | |
| Aldehyde C-12 Lauric | 9.50 | 9.00 | 9.00 | 8.00 |
| Aldehyde C-12 MNA | 9.00 | 8.50 | 7.25 | 8.00 |
| Aldehyde C14 | 10.00 | 9.00 | 8.00 | 8.25 |
| (Undecalactone γ) | | | | |
| Aldehyde Mandarine | 10.00 | 10.00 | 10.00 | 10.00 |
| Allyl Amyl Glycolate | 10.00 | 10.00 | 9.25 | 8.25 |
| Ambrinol | 10.00 | 9.00 | 7.50 | 7.25 |
| Ambrofix | 10.00 | 10.00 | 10.00 | 10.00 |
| Ambrox Super | 7.00 | 10.00 | 7.00 | 6.00 |
| (Ambroxan) | | | | |
| Anethole | 7.25 | 9.00 | 7.00 | 6.00 |
| Anisic Aldehyde | 9.00 | 9.00 | 9.00 | 9.00 |
| Anisyl Acetate | 9.00 | 9.00 | 9.00 | 9.00 |
| Bacdanol | 9.00 | 10.00 | 8.00 | 8.00 |
| Benzyl Acetate | 8.00 | 8.00 | 8.00 | 8.00 |
| Caryophyllene | 6.00 | 10.00 | 6.25 | 7.25 |
| Cashmeran (S51) | 10.00 | 10.00 | 10.00 | 7.00 |
| Cashmeran (S55) | 10.00 | 10.00 | 9.00 | 8.00 |
| Cetalox/Ambrox | 10.00 | 10.00 | 10.00 | 10.00 |
| Cinnamalva | 10.00 | 10.00 | 8.00 | 9.00 |
| Citral | 10.00 | 9.00 | 7.00 | 9.00 |
| Clearwood | 8.00 | 8.00 | 7.00 | 7.00 |
| Cyclal C/Triplal | 9.00 | 10.00 | 8.00 | 7.00 |
| Damascenone | 10.00 | 10.00 | 10.00 | 10.00 |
| Dibenzyl Ether | 5.00 | 8.00 | 7.00 | 7.00 |
| Dihydro Eugenol | 10.00 | 10.00 | 8.00 | 9.00 |
| Dihydro Ionone Beta | 10.00 | 10.00 | 10.00 | 10.00 |
| Dihydro Myrcenol | 9.00 | 10.00 | 9.00 | 9.00 |
| Dimetol | 10.00 | 8.00 | 9.00 | 9.00 |
| Ethyl Amyl Ketone | 10.00 | 10.00 | 10.00 | 10.00 |
| Ethyl Vanillin | 9.00 | 10.00 | 9.00 | 7.00 |
| Ethylene Brassylate | 8.00 | 9.00 | 7.25 | 7.25 |
| Eucalyptol | 10.00 | 10.00 | 8.00 | 9.00 |
| Evernyl | 8.00 | 8.00 | 7.50 | 7.50 |
| Fennaldehyde | 10.00 | 10.00 | 10.00 | 10.00 |
| Fraistone | 10.00 | 10.00 | 10.00 | 10.00 |
| Fructone | 7.00 | 9.00 | 9.00 | 8.00 |
| Galaxolide | 7.25 | 10.00 | 6.00 | 6.00 |
| Grisalva | 8.00 | 9.00 | 7.00 | 8.00 |
| Hedione | 8.00 | 8.00 | 7.00 | 6.00 |
| Heliotropine | 10.00 | 10.00 | 9.00 | 9.00 |
| Cis-3-Hexenol | 10.00 | 10.00 | 10.00 | 10.00 |
| Cis-3-Hexenyl | 6.75 | 8.75 | 7.75 | 7.00 |
| Salicylate | | | | |
| Cis-3-Hexenyl Tiglate | 10.00 | 10.00 | 10.00 | 10.00 |
| Humus Ether | 10.00 | 10.00 | 10.00 | 9.00 |
| Iso E Super | 7.25 | 9.00 | 6.00 | 6.00 |
| Isoamyl Acetate | 9.00 | 10.00 | 9.00 | 8.00 |
| Isobornyl Acetate | 10.00 | 10.00 | 10.00 | 10.00 |
| Lime Oxide | 10.00 | 10.00 | 10.00 | 10.00 |
| Linalool | 9.00 | 9.00 | 8.00 | 7.00 |
| Linalyl Acetate | 8.00 | 10.00 | 8.00 | 7.00 |
| Methyl Cinnamate | 10.00 | 10.00 | 10.00 | 10.00 |
| Methyl Diantilis | 10.00 | 10.00 | 10.00 | 10.00 |
| Methyl Geosmin | 10.00 | 10.00 | 10.00 | 10.00 |
| Methyl Hexyl Ketone | 10.00 | 10.00 | 10.00 | 10.00 |
| Methyl Ionone Gamma | 7.25 | 10.00 | 7.00 | 6.00 |
| Muscenone | 8.00 | 9.00 | 8.00 | 8.00 |
| Nootkatone | 10.00 | 9.00 | 7.00 | 7.00 |
| Olibanol | 10.00 | 10.00 | 10.00 | 10.00 |
| Paradisamide | 8.75 | 9.00 | 7.00 | 8.00 |
| Precyclemone B | 10.00 | 10.00 | 10.00 | 10.00 |
| Terpinyl Acetate | 8.00 | 10.00 | 8.00 | 7.00 |
| Toscanol | 10.00 | 10.00 | 10.00 | 10.00 |
| Vanillin | 10.00 | 10.00 | 9.00 | 9.00 |
| Vertofix Coeur | 8.00 | 9.00 | 7.00 | 7.00 |
| Average | 9.05 | 9.53 | 8.53 | 8.37 |

TABLE 4 shows the odor value for each of the different concentrations of fragrance ingredients in bioderived 1,3-Butylene Glycol (bioBG) diluent (i.e., 0.01%, 0.1%, 1%, 10%) of the blotter comparison analysis. Not determinable (ND).

TABLE 4

| | 0.01% | 0.1% | 1% | 10% | Average | |
|---|---|---|---|---|---|---|
| INGREDIENT | bioBG | bioBG | bioBG | bioBG | Rating | Comments |
| Aldehyde C-10 | 9.00 | 9.00 | 9.00 | 9.00 | 9.00 | Clear soluble, good |
| | | | | | | character, strong |
| Aldehyde C-11 Undecylenic | 9.00 | 9.00 | 9.00 | 9.00 | 9.00 | Very Slight haze, |
| | | | | | | Clear w/heat, strong, |
| Aldehyde C-12 Lauric | 10.00 | 10.00 | 9.00 | 9.00 | 9.50 | Very Slight haze, |
| | | | | | | Clear w/heat, strong, |
| Aldehyde C-12 MNA | 9.00 | 9.00 | 9.00 | 9.00 | 9.00 | Enhanced odor |
| | | | | | | character (stronger) |
| Aldehyde C14 | 10.00 | ND | ND | ND | 10.00 | Consistent and Strong |
| (Undecalactone γ) | | | | | | |
| Aldehyde Mandarine | 10.00 | 10.00 | 10.00 | 10.00 | 10.00 | |
| Allyl Amyl Glycolate | 10.00 | ND | ND | ND | 10.00 | Character consistent |
| | | | | | | with neat |
| Ambrinol | 8.00 | 8.00 | 8.00 | 4.00 | 7.00 | Character consistent |
| | | | | | | with neat, strong |
| Ambrofix | 10.00 | ND | ND | ND | 10.00 | |
| Ambrox Super (Ambroxan) | 9.00 | 9.00 | 9.00 | 5.00 | 8.00 | Insoluble @10% |
| Anethole | 8.00 | 8.00 | 8.00 | 5.00 | 7.25 | Slightly altered |
| | | | | | | sweeter |
| Anisic Aldehyde | 9.00 | 9.00 | 9.00 | 9.00 | 9.00 | Consistent and strong |
| Anisyl Acetate | 9.00 | 9.00 | 9.00 | 9.00 | 9.00 | Consistent and strong |
| Bacdanol | 10.00 | ND | ND | ND | 10.00 | Good character, |
| | | | | | | slightly less strong |
| Benzyl Acetate | 10.00 | ND | ND | ND | 10.00 | Good character, |
| | | | | | | slightly less strong |
| Caryophyllene | 9.00 | 9.00 | 9.00 | 9.00 | 9.00 | |
| Cashmeran | 10.00 | 10.00 | 10.00 | 10.00 | 10.00 | |
| Cashmeran | 10.00 | 10.00 | 10.00 | 10.00 | 10.00 | Character consistent |
| | | | | | | with neat |
| Cetalox/Ambrox | 10.00 | 10.00 | 10.00 | 10.00 | 10.00 | |
| Cinnamalva | 10.00 | 10.00 | 10.00 | 10.00 | 10.00 | Strong |
| Citral | 10.00 | 10.00 | 10.00 | 10.00 | 10.00 | Excellent clarity, |
| | | | | | | consistent with neat |
| Clearwood | 9.00 | 9.00 | 9.00 | 9.00 | 9.00 | |
| Cyclal C/Triplal | 10.00 | ND | ND | ND | 10.00 | |
| Damascenone | 10.00 | 10.00 | 10.00 | 10.00 | 10.00 | Good character, |
| | | | | | | slightly less strong |
| Dibenzyl Ether | 10.00 | ND | ND | ND | 10.00 | Insoluble @10% |
| Dihydro Eugenol | 8.00 | 8.00 | 8.00 | 8.00 | 8.00 | |
| Dihydro Ionone Beta | 10.00 | 10.00 | 10.00 | 10.00 | 10.00 | |
| Dihydro Myrcenol | 10.00 | ND | ND | ND | 10.00 | Excellent clarity, |
| | | | | | | consistent with neat |
| Dimetol | 10.00 | ND | ND | ND | 10.00 | Excellent clarity, |
| | | | | | | consistent with neat |
| Ethyl Amyl Ketone | 5.00 | 5.00 | 5.00 | 5.00 | 5.00 | Excellent clarity, |
| | | | | | | consistent with neat |
| Ethyl Vanillin | 8.00 | 8.00 | 8.00 | 8.00 | 8.00 | Good solubility with |
| | | | | | | heat applied |
| Ethylene Brassylate | 10.00 | ND | ND | ND | 10.00 | |
| Eucalyptol | 8.00 | 8.00 | 8.00 | 8.00 | 8.00 | Excellent clarity, |
| | | | | | | consistent with neat |
| Evernyl | 9.00 | 9.00 | 9.00 | 9.00 | 9.00 | Good character, |
| | | | | | | slightly less strong |
| Fennaldehyde | 8.00 | 8.00 | 8.00 | 5.00 | 7.25 | |
| Fraistone | 10.00 | 10.00 | 10.00 | 10.00 | 10.00 | |
| Fructone | 10.00 | ND | ND | ND | 10.00 | Altered, muted |
| Galaxolide | 7.00 | 7.00 | 7.00 | 7.00 | 7.00 | No solubility at room |
| | | | | | | temp |
| Grisalva | 9.00 | 9.00 | 9.00 | 9.00 | 9.00 | Insoluble, strong |
| Hedione | 10.00 | 10.00 | 10.00 | 10.00 | 10.00 | Good character, |
| | | | | | | slightly less strong |
| Heliotropine | 9.00 | 9.00 | 9.00 | 9.00 | 9.00 | Excellent clarity, |
| | | | | | | consistent with neat |
| Cis-3-Hexenol | 10.00 | 10.00 | 10.00 | 5.00 | 8.75 | Strong |
| Cis-3-Hexenyl Salicylate | 10.00 | 10.00 | 10.00 | 10.00 | 10.00 | |
| Hexenyl Tiglate, Cis-3- | 7.00 | 7.00 | 8.00 | 5.00 | 6.75 | |
| Humus Ether | 10.00 | ND | ND | ND | 10.00 | Excellent clarity, |
| | | | | | | consistent with neat |
| iso Amyl Acetate | 10.00 | 10.00 | 10.00 | 10.00 | 10.00 | Consistent and strong |
| Iso E Super | 10.00 | ND | ND | ND | 10.00 | Strong Insoluble |
| Isobornyl Acetate | 10.00 | 10.00 | 10.00 | 10.00 | 10.00 | Excellent clarity, |
| | | | | | | consistent with neat |
| Lime Oxide | 9.00 | 9.00 | 9.00 | 5.00 | 8.00 | |
| Linalool | 9.00 | 9.00 | 9.00 | 5.00 | 8.00 | Equilvalent strength |
| | | | | | | to DPG |
| Linalyl Acetate | 10.00 | ND | ND | ND | 10.00 | Character consistent |
| | | | | | | with neat |
| Methyl Cinnamate | 9.00 | 9.00 | 9.00 | 5.00 | 8.00 | |
| Methyl Diantilis | 8.00 | 8.00 | 8.00 | 5.00 | 7.25 | |
| Methyl Geosmin | 9.00 | 9.00 | 9.00 | 5.00 | 8.00 | |
| Methyl Hexyl Ketone | 10.00 | 10.00 | 10.00 | 10.00 | 10.00 | |
| Methyl Ionone Gamma | 6.00 | 6.00 | 7.00 | 5.00 | 6.00 | Character consistent |
| | | | | | | with neat less strong |
| Muscenone | 10.00 | 10.00 | 10.00 | 10.00 | 10.00 | |
| Nootkatone | 10.00 | 10.00 | 10.00 | 10.00 | 10.00 | Excellent clarity, |
| | | | | | | consistent with neat |
| Olibanol | 10.00 | ND | ND | ND | 10.00 | Excellent clarity, |
| | | | | | | consistent with neat |
| Paradisamide | 10.00 | ND | ND | ND | 10.00 | Consistent but not as |
| | | | | | | strong |
| Precyclemone B | 9.00 | 9.00 | 9.00 | 9.00 | 9.00 | |
| Terpinyl Acetate | 10.00 | ND | ND | ND | 10.00 | Character consistent |
| | | | | | | with neat |
| Toscanol | 9.00 | 9.00 | 9.00 | 5.00 | 8.00 | |
| Vanillin | 8.00 | 8.00 | 8.00 | 5.00 | 7.25 | Excellent clarity, |
| | | | | | | consistent with neat |
| Vertofix Coeur | 9.00 | 9.00 | 9.00 | 5.00 | 8.00 | Strong Insoluble |
| Average | 9.25 | 8.98 | 9.00 | 7.88 | 9.05 | |

TABLE 5 shows the odor value for each of the different concentrations of fragrance ingredients in dipropylene glycol (DPG) diluent (i.e., 0.01%, 0.1%, 1%, 10%) of the blotter comparison analysis. Not determinable (ND).

TABLE 5

| | 0.01% | 0.1% | 1% | 10% | Average | |
|---|---|---|---|---|---|---|
| INGREDIENT | DPG | DPG | DPG | DPG | Rating | Comments |
| Aldehyde C-10 | 10.00 | 10.00 | 10.00 | 10.00 | 10.00 | Good consistency |
| | | | | | | and strength |
| Aldehyde C-11 Undecylenic | 10.00 | 10.00 | 10.00 | 10.00 | 10.00 | Good consistency |
| | | | | | | and strength |
| Aldehyde C-12 Lauric | 9.00 | 9.00 | 9.00 | 9.00 | 9.00 | Good consistency |
| | | | | | | and strength |
| Aldehyde C-12 MNA | 8.00 | 8.00 | 9.00 | 9.00 | 8.50 | Slightly less power- |
| | | | | | | ful than in BioBG |
| Aldehyde C14 | 10.00 | ND | ND | ND | 10.00 | |
| (Undecalactone γ) | | | | | | |
| Aldehyde Mandarine | 9.00 | 9.00 | 9.00 | 9.00 | 9.00 | Good consistency |
| | | | | | | slightly less strong |
| Allyl Amyl Glycolate | 10.00 | ND | ND | ND | 10.00 | |
| Ambrinol | 10.00 | 10.00 | 10.00 | 10.00 | 10.00 | Good consistency |
| | | | | | | and strength |
| Ambrofix | 10.00 | ND | ND | ND | 10.00 | Good consistency |
| | | | | | | and strength |
| Ambrox Super (Ambroxan) | 9.00 | 9.00 | 9.00 | 9.00 | 9.00 | Good consistency |
| | | | | | | and strength |
| Anethole | 9.00 | 9.00 | 9.00 | 9.00 | 9.00 | Good consistency |
| | | | | | | and strength |
| Anisic Aldehyde | 9.00 | 9.00 | 9.00 | 9.00 | 9.00 | Good consistency |
| | | | | | | and strength |
| Anisyl Acetate | 9.00 | 9.00 | 9.00 | 9.00 | 9.00 | Good consistency |
| | | | | | | and strength |
| Bacdanol | 10.00 | ND | ND | ND | 10.00 | |
| Benzyl Acetate | 10.00 | ND | ND | ND | 10.00 | |
| Caryophyllene | 10.00 | 10.00 | 10.00 | 10.00 | 10.00 | Good consistency |
| | | | | | | and strength |
| Cashmeran | 10.00 | 10.00 | 10.00 | 10.00 | 10.00 | Good consistency |
| | | | | | | and strength |
| Cashmeran | 10.00 | 10.00 | 10.00 | 10.00 | 10.00 | Good consistency |
| | | | | | | and strength |
| Cetalox/Ambrox | 10.00 | 10.00 | 10.00 | 10.00 | 10.00 | Good consistency |
| | | | | | | and strength |
| Cinnamalva | 10.00 | 10.00 | 10.00 | 10.00 | 10.00 | Good consistency |
| | | | | | | and strength |
| Citral | 9.00 | 9.00 | 9.00 | 9.00 | 9.00 | Good consistency |
| | | | | | | and strength |
| Clearwood | 10.00 | 10.00 | 10.00 | 10.00 | 10.00 | Good consistency |
| | | | | | | and strength |
| Cyclal C/Triplal | 10.00 | ND | ND | ND | 10.00 | |
| Damascenone | 9.00 | 9.00 | 9.00 | 9.00 | 9.00 | Good consistency |
| | | | | | | slightly less strong |
| Dibenzyl Ether | 10.00 | ND | ND | ND | 10.00 | |
| Dihydro Eugenol | 8.00 | 8.00 | 8.00 | 8.00 | 8.00 | Yellowing |
| Dihydro Ionone Beta | 10.00 | 10.00 | 10.00 | 10.00 | 10.00 | Good consistency |
| | | | | | | and strength |
| Dihydro Myrcenol | 10.00 | ND | ND | ND | 10.00 | |
| Dimetol | 10.00 | ND | ND | ND | 10.00 | |
| Ethyl Amyl Ketone | 8.00 | 8.00 | 8.00 | 8.00 | 8.00 | Muted |
| Ethyl Vanillin | 8.00 | 8.00 | 8.00 | 8.00 | 8.00 | Good character, |
| | | | | | | slightly less strong |
| Ethylene Brassylate | 10.00 | ND | ND | ND | 10.00 | Good character, |
| | | | | | | slightly less strong |
| Eucalyptol | 8.00 | 8.00 | 8.00 | 8.00 | 8.00 | Good character, |
| | | | | | | slightly less strong |
| Evernyl | 9.00 | 9.00 | 9.00 | 9.00 | 9.00 | Good consistency |
| | | | | | | and strength |
| Fennaldehyde | 10.00 | 10.00 | 10.00 | 10.00 | 10.00 | Good consistency |
| | | | | | | and strength |
| Fraistone | 10.00 | 10.00 | 10.00 | 10.00 | 10.00 | Consistent |
| Fructone | 10.00 | ND | ND | ND | 10.00 | |
| Galaxolide | 9.00 | 9.00 | 9.00 | 9.00 | 9.00 | Good strength |
| Grisalva | 10.00 | 10.00 | 10.00 | 10.00 | 10.00 | Good consistency |
| | | | | | | and strength |
| Hedione | 9.00 | 9.00 | 9.00 | 9.00 | 9.00 | Good strength |
| Heliotropine | 10.00 | 10.00 | 10.00 | 10.00 | 10.00 | |
| Cis-3-Hexenol | 9.00 | 9.00 | 9.00 | 9.00 | 9.00 | Good strength |
| Cis-3-Hexenyl Salicylate | 10.00 | 10.00 | 10.00 | 10.00 | 10.00 | Good consistency |
| | | | | | | and strength |
| Cis-3-Hexenyl Tiglate | 9.00 | 9.00 | 9.00 | 8.00 | 8.75 | Good consistency |
| | | | | | | and strength |
| Humus Ether | 10.00 | ND | ND | ND | 10.00 | |
| iso Amyl Acetate | 8.00 | 8.00 | 8.00 | 8.00 | 8.00 | Slightly muted |
| Iso E Super | 10.00 | ND | ND | ND | 10.00 | |
| Isobornyl Acetate | 10.00 | 10.00 | 10.00 | 10.00 | 10.00 | Good consistency |
| | | | | | | and strength |
| Lime Oxide | 10.00 | 10.00 | 10.00 | 10.00 | 10.00 | Good consistency |
| | | | | | | and strength |
| Linalool | 10.00 | 10.00 | 10.00 | 10.00 | 10.00 | Good consistency |
| | | | | | | and strength |
| Linalyl Acetate | 10.00 | ND | ND | ND | 10.00 | |
| Methyl Cinnamate | 9.00 | 9.00 | 9.00 | 9.00 | 9.00 | |
| Methyl Diantilis | 10.00 | 10.00 | 10.00 | 10.00 | 10.00 | Good solubility |
| Methyl Geosmin | 9.00 | 9.00 | 9.00 | 9.00 | 9.00 | |
| Methyl Hexyl Ketone | 10.00 | 10.00 | 10.00 | 10.00 | 10.00 | Good consistency |
| | | | | | | and strength |
| Methyl Ionone Gamma | 10.00 | 10.00 | 10.00 | 10.00 | 10.00 | |
| Muscenone | 10.00 | 10.00 | 10.00 | 10.00 | 10.00 | Strong |
| Nootkatone | 10.00 | 10.00 | 10.00 | 10.00 | 10.00 | |
| Olibanol | 10.00 | ND | ND | ND | 10.00 | |
| Paradisamide | 10.00 | ND | ND | ND | 10.00 | |
| Precyclemone B | 10.00 | 10.00 | 10.00 | 10.00 | 10.00 | Consistent and strong |
| Terpinyl Acetate | 10.00 | ND | ND | ND | 10.00 | |
| Toscanol | 8.00 | 8.00 | 8.00 | 8.00 | 8.00 | |
| Vanillin | 9.00 | 9.00 | 9.00 | 9.00 | 9.00 | Good consistency |
| | | | | | | and strength |
| Vertofix Coeur | 9.00 | 9.00 | 9.00 | 9.00 | 9.00 | Good consistency |
| | | | | | | and strength |
| Average | 9.52 | 9.35 | 9.38 | 9.35 | 9.53 | |

TABLE 6 shows the odor value for each of the different concentrations of fragrance ingredients in benzyl benzoate (BB) diluent (i.e., 0.01%, 0.1%, 1%, 10%) of the blotter comparison analysis. Not determinable (ND).

TABLE 6

| | 0.01% | 0.1% | 1% | 10% | Average | |
|---|---|---|---|---|---|---|
| INGREDIENT | BB | BB | BB | BB | Rating | Comments |
| Aldehyde C-10 | 7.00 | 7.00 | 7.00 | 7.00 | 7.00 | Muted |
| Aldehyde C-11 Undecylenic | 9.00 | 9.00 | 9.00 | 9.00 | 9.00 | Good strength |
| Aldehyde C-12 Lauric | 9.00 | 9.00 | 9.00 | 9.00 | 9.00 | Good strength |
| Aldehyde C-12 MNA | 7.00 | 7.00 | 7.00 | 8.00 | 7.25 | Least strength character |
| | | | | | | good, slightly muted |
| Aldehyde C14 | 8.00 | 8.00 | 8.00 | 8.00 | 8.00 | Slightly less strong |
| (Undecalactone γ) | | | | | | |
| Aldehyde Mandarine | 10.00 | ND | ND | ND | 10.00 | |
| Allyl Amyl Glycolate | 9.00 | 9.00 | 9.00 | 10.00 | 9.25 | Slightly altered |
| Ambrinol | 7.00 | 7.00 | 8.00 | 8.00 | 7.50 | Slightly muted |
| Ambrofix | 10.00 | ND | ND | ND | 10.00 | |
| Ambrox Super (Ambroxan) | 7.00 | 7.00 | 7.00 | 7.00 | 7.00 | Muted |
| Anethole | 7.00 | 7.00 | 7.00 | 7.00 | 7.00 | Muted |
| Anisic Aldehyde | 9.00 | 9.00 | 9.00 | 9.00 | 9.00 | Consistent and strong |
| Anisyl Acetate | 9.00 | 9.00 | 9.00 | 9.00 | 9.00 | Consistent and strong |
| Bacdanol | 8.00 | 8.00 | 8.00 | 8.00 | 8.00 | |
| Benzyl Acetate | 8.00 | 8.00 | 8.00 | 8.00 | 8.00 | Good character, |
| | | | | | | slightly less strong |
| Caryophyllene | 6.00 | 6.00 | 6.00 | 7.00 | 6.25 | |
| Cashmeran (S51) | 10.00 | ND | ND | ND | 10.00 | |
| Cashmeran (S55) | 9.00 | 9.00 | 9.00 | 9.00 | 9.00 | Good strength |
| Cetalox/Ambrox | 10.00 | ND | ND | ND | 10.00 | Muted |
| Cinnamalva | 8.00 | 8.00 | 8.00 | 8.00 | 8.00 | Slightly muted |
| Citral | 7.00 | 7.00 | 7.00 | 7.00 | 7.00 | Muted |
| Clearwood | 7.00 | 7.00 | 7.00 | 7.00 | 7.00 | |
| Cyclal C/Triplal | 8.00 | 8.00 | 8.00 | 8.00 | 8.00 | |
| Damascenone | 10.00 | ND | ND | ND | 10.00 | |
| Dibenzyl Ether | 7.00 | 7.00 | 7.00 | 7.00 | 7.00 | Muted |
| Dihydro Eugenol | 8.00 | 8.00 | 8.00 | 8.00 | 8.00 | |
| Dihydro Ionone Beta | 10.00 | ND | ND | ND | 10.00 | |
| Dihydro Myrcenol | 9.00 | 9.00 | 9.00 | 9.00 | 9.00 | Good strength |
| Dimetol | 9.00 | 9.00 | 9.00 | 9.00 | 9.00 | Good strength |
| Ethyl Amyl Ketone | 10.00 | 10.00 | 10.00 | 10.00 | 10.00 | |
| Ethyl Vanillin | 9.00 | 9.00 | 9.00 | 9.00 | 9.00 | Good character, |
| | | | | | | slightly less strong |
| Ethylene Brassylate | 7.00 | 7.00 | 7.00 | 8.00 | 7.25 | |
| Eucalyptol | 8.00 | 8.00 | 8.00 | 8.00 | 8.00 | Slightly muted |
| Evernyl | 8.00 | 8.00 | 8.00 | 6.00 | 7.50 | Poor solubility |
| Fennaldehyde | 10.00 | ND | ND | ND | 10.00 | |
| Fraistone | 10.00 | ND | ND | ND | 10.00 | |
| Fructone | 9.00 | 9.00 | 9.00 | 9.00 | 9.00 | Good strength |
| Galaxolide | 6.00 | 6.00 | 6.00 | 6.00 | 6.00 | Suppressed, |
| | | | | | | solubility good |
| Grisalva | 7.00 | 7.00 | 7.00 | 7.00 | 7.00 | Muted |
| Hedione | 7.00 | 7.00 | 7.00 | 7.00 | 7.00 | Slightly muted |
| Heliotropine | 9.00 | 9.00 | 9.00 | 9.00 | 9.00 | Good character, |
| | | | | | | slightly less strong |
| Cis-3-Hexenol | 10.00 | 10.00 | 10.00 | 10.00 | 10.00 | |
| Cis-3-Hexenyl Salicylate | 8.00 | 8.00 | 8.00 | 7.00 | 7.75 | |
| Cis-3-Hexenyl Tiglate | 10.00 | ND | ND | ND | 10.00 | |
| Humus Ether | 10.00 | 10.00 | 10.00 | 10.00 | 10.00 | Good strength |
| Iso E Super | 9.00 | 9.00 | 9.00 | 9.00 | 9.00 | Good strength |
| Isoamyl Acetate | 6.00 | 6.00 | 6.00 | 6.00 | 6.00 | Muted |
| Isobornyl Acetate | 10.00 | ND | ND | ND | 10.00 | Good character, |
| | | | | | | slightly less strong |
| Lime Oxide | 10.00 | ND | ND | ND | 10.00 | |
| Linalool | 8.00 | 8.00 | 8.00 | 8.00 | 8.00 | Slightly muted |
| Linalyl Acetate | 8.00 | 8.00 | 8.00 | 8.00 | 8.00 | Slightly muted |
| Methyl Cinnamate | 10.00 | ND | ND | ND | 10.00 | |
| Methyl Diantilis | 10.00 | ND | ND | ND | 10.00 | |
| Methyl Geosmin | 10.00 | ND | ND | ND | 10.00 | |
| Methyl Hexyl Ketone | 10.00 | ND | ND | ND | 10.00 | |
| Methyl Ionone Gamma | 7.00 | 7.00 | 7.00 | 7.00 | 7.00 | Muted |
| Muscenone | 8.00 | 8.00 | 8.00 | 8.00 | 8.00 | |
| Nootkatone | 7.00 | 7.00 | 7.00 | 7.00 | 7.00 | Muted |
| Olibanol | 10.00 | ND | ND | ND | 10.00 | Good character, |
| | | | | | | slightly less strong |
| Paradisamide | 7.00 | 7.00 | 7.00 | 7.00 | 7.00 | Slightly muted |
| Precyclemone B | 10.00 | ND | ND | ND | 10.00 | |
| Terpinyl Acetate | 8.00 | 8.00 | 8.00 | 8.00 | 8.00 | Slightly muted |
| Toscanol | 10.00 | ND | ND | ND | 10.00 | |
| Vanillin | 9.00 | 9.00 | 9.00 | 9.00 | 9.00 | Good character, slightly |
| | | | | | | less strong, pinking |
| Vertofix Coeur | 7.00 | 7.00 | 7.00 | 7.00 | 7.00 | Altered |
| Average | 8.52 | 7.96 | 7.98 | 8.00 | 8.53 | Muted |

TABLE 7 shows the odor value for each of the different concentrations of fragrance ingredients in isopropyl myristate (IPM) diluent (i.e., 0.01%, 0.1%, 1%, 10%) of the blotter comparison analysis. Not determinable (ND).

TABLE 7

| | 0.01% | 0.1% | 1% | 10% | Average | |
|---|---|---|---|---|---|---|
| INGREDIENT | IPM | IPM | IPM | IPM | Rating | Comments |
| Aldehyde C-10 | 8.00 | 8.00 | 8.00 | 8.00 | 8.00 | Good character, |
| | | | | | | slightly less strong |
| Aldehyde C-11 Undecylenic | 8.00 | 8.00 | 8.00 | 8.00 | 8.00 | Good character, |
| | | | | | | slightly less strong |
| Aldehyde C-12 Lauric | 8.00 | 8.00 | 8.00 | 8.00 | 8.00 | Good character, |
| | | | | | | slightly less strong |
| Aldehyde C-12 MNA | 8.00 | 8.00 | 8.00 | 8.00 | 8.00 | Good character, |
| | | | | | | slightly less strong |
| Aldehyde C14 | 8.00 | 8.00 | 8.00 | 9.00 | 8.25 | Good strength |
| (Undecalactone γ) | | | | | | |
| Aldehyde Mandarine | 10.00 | ND | ND | ND | 10.00 | |
| Allyl Amyl Glycolate | 8.00 | 8.00 | 8.00 | 9.00 | 8.25 | Muted, Altered |
| Ambrinol | 7.00 | 7.00 | 7.00 | 8.00 | 7.25 | Less strong |
| Ambrofix | 10.00 | ND | ND | ND | 10.00 | |
| Ambrox Super (Ambroxan) | 6.00 | 6.00 | 6.00 | 6.00 | 6.00 | Suppressed |
| Anethole | 6.00 | 6.00 | 6.00 | 6.00 | 6.00 | Suppressed |
| Anisic Aldehyde | 9.00 | 9.00 | 9.00 | 9.00 | 9.00 | Consistent and strong |
| Anisyl Acetate | 9.00 | 9.00 | 9.00 | 9.00 | 9.00 | Consistent and strong |
| Bacdanol | 8.00 | 8.00 | 8.00 | 8.00 | 8.00 | Good strength |
| Benzyl Acetate | 8.00 | 8.00 | 8.00 | 8.00 | 8.00 | Good character, |
| | | | | | | slightly less strong |
| Caryophyllene | 7.00 | 7.00 | 7.00 | 8.00 | 7.25 | |
| Cashmeran (S51) | 7.00 | 7.00 | 7.00 | 7.00 | 7.00 | |
| Cashmeran (S55) | 8.00 | 8.00 | 8.00 | 8.00 | 8.00 | Slightly altered, |
| | | | | | | strong |
| Cetalox/Ambrox | 10.00 | ND | ND | ND | 10.00 | |
| Cinnamalva | 9.00 | 9.00 | 9.00 | 9.00 | 9.00 | Good strength |
| Citral | 9.00 | 9.00 | 9.00 | 9.00 | 9.00 | Good strength, |
| | | | | | | slightly less strong |
| Clearwood | 7.00 | 7.00 | 7.00 | 7.00 | 7.00 | Good strength |
| Cyclal C/Triplal | 7.00 | 7.00 | 7.00 | 7.00 | 7.00 | |
| Damascenone | 10.00 | ND | ND | ND | 10.00 | |
| Dibenzyl Ether | 7.00 | 7.00 | 7.00 | 7.00 | 7.00 | Muted |
| Dihydro Eugenol | 9.00 | 9.00 | 9.00 | 9.00 | 9.00 | Good strength |
| Dihydro Ionone Beta | 10.00 | ND | ND | ND | 10.00 | Good strength |
| Dihydro Myrcenol | 9.00 | 9.00 | 9.00 | 9.00 | 9.00 | Good strength, |
| | | | | | | slightly less strong |
| Dimetol | 9.00 | 9.00 | 9.00 | 9.00 | 9.00 | Good strength |
| Ethyl Amyl Ketone | 10.00 | ND | ND | ND | 10.00 | |
| Ethyl Vanillin | 7.00 | 7.00 | 7.00 | 7.00 | 7.00 | Insoluble room temp |
| Ethylene Brassylate | 7.00 | 7.00 | 7.00 | 8.00 | 7.25 | |
| Eucalyptol | 9.00 | 9.00 | 9.00 | 9.00 | 9.00 | Slightly muted |
| Evernyl | 8.00 | 8.00 | 8.00 | 6.00 | 7.50 | Moderate solubility |
| Fennaldehyde | 10.00 | ND | ND | ND | 10.00 | |
| Fraistone | 10.00 | ND | ND | ND | 10.00 | |
| Fructone | 8.00 | 8.00 | 8.00 | 8.00 | 8.00 | Slightly muted |
| Galaxolide | 6.00 | 6.00 | 6.00 | 6.00 | 6.00 | Suppressed, Solubility, |
| | | | | | | goes in with heating |
| Grisalva | 8.00 | 8.00 | 8.00 | 8.00 | 8.00 | |
| Hedione | 6.00 | 6.00 | 6.00 | 6.00 | 6.00 | Muted |
| Heliotropine | 9.00 | 9.00 | 9.00 | 9.00 | 9.00 | Good strength, |
| | | | | | | slightly less strong |
| Cis-3-Hexenol | 10.00 | 10.00 | 10.00 | 10.00 | 10.00 | |
| Cis-3-Hexenyl Salicylate | 7.00 | 7.00 | 7.00 | 7.00 | 7.00 | |
| Cis-3-Hexenyl Tiglate | 10.00 | ND | ND | ND | 10.00 | |
| Humus Ether | 9.00 | 9.00 | 9.00 | 9.00 | 9.00 | |
| Iso E Super | 6.00 | 6.00 | 6.00 | 6.00 | 6.00 | Muted |
| Isoamyl Acetate | 8.00 | 8.00 | 8.00 | 8.00 | 8.00 | Good character, |
| | | | | | | slightly less strong |
| Isobornyl Acetate | 10.00 | ND | ND | ND | 10.00 | Good strength, |
| | | | | | | slightly less strong |
| Lime Oxide | 10.00 | ND | ND | ND | 10.00 | |
| Linalool | 7.00 | 7.00 | 7.00 | 7.00 | 7.00 | Muted |
| Linalyl Acetate | 7.00 | 7.00 | 7.00 | 7.00 | 7.00 | Muted |
| Methyl Cinnamate | 10.00 | ND | ND | ND | 10.00 | Good strength |
| Methyl Diantilis | 10.00 | ND | ND | ND | 10.00 | Good strength |
| Methyl Geosmin | 10.00 | ND | ND | ND | 10.00 | |
| Methyl Hexyl Ketone | 10.00 | ND | ND | ND | 10.00 | |
| Methyl Ionone Gamma | 6.00 | 6.00 | 6.00 | 6.00 | 6.00 | Suppressed |
| Muscenone | 8.00 | 8.00 | 8.00 | 8.00 | 8.00 | |
| Nootkatone | 7.00 | 7.00 | 7.00 | 7.00 | 7.00 | Muted |
| Olibanol | 10.00 | ND | ND | ND | 10.00 | Good strength, |
| | | | | | | slightly less strong |
| Paradisamide | 8.00 | 8.00 | 8.00 | 8.00 | 8.00 | Slightly muted |
| Precyclemone B | 10.00 | ND | ND | ND | 10.00 | |
| Terpinyl Acetate | 7.00 | 7.00 | 7.00 | 7.00 | 7.00 | Muted |
| Toscanol | 10.00 | ND | ND | ND | 10.00 | |
| Vanillin | 9.00 | 9.00 | 9.00 | 9.00 | 9.00 | Slightly less strong, |
| | | | | | | heat for solubility |
| Vertofix Coeur | 7.00 | 7.00 | 7.00 | 7.00 | 7.00 | Altered |
| Average | 8.35 | 7.72 | 7.72 | 7.79 | 8.37 | |

Various characteristics of bioderived 1,3-butylene glycol (bioBG) were determined. The different fragrance ingredients were tested for solubility in bioderived 1,3-BG at 10%, 1%, 0.1%, and 0.01% and characterized as excellent, good, moderate, poor, or none. Clarity for each of the fragrance ingredients (10% in bioderived 1,3-BG) was determined by visual observations after compounding and blending the fragrance ingredient with bioderived 1,3-BG and identified as clear, slightly haze, cloudy, particles, or separation. Headspace for each of the fragrance ingredients (at 10% in bioderived 1,3-BG) was determined by organoleptic evaluation of the diluted fragrance ingredients in each of the dilutions through qualitative analysis. The headspace for each was identified as bright, moderate, or suppressed. The odors of each of the fragrance ingredients at 10% in bioderived 1,3-BG have been classified.

TABLE 8 shows various characteristics of a 10% concentration of fragrance ingredients in bioderived 1,3-BG diluent. In particular, the odor classification, solubility, clarity, and headspace of each of the listed fragrance ingredients was evaluated. Not determinable (ND).

TABLE 8

| | Odor | Solu- | | |
|---|---|---|---|---|
| | Classifi- | bility | Clarity | Headspace |
| Fragrance Ingredient | cation | 10% | 10% | 10% |
| Aldehyde C-10 | Aldehydic | Good | Slightly | Bright |
| | | | Haze | |
| Aldehyde C-11 | Aldehydic | Moderate | Slightly | Bright |
| Undecylenic | | | Haze | |
| Aldehyde C-12 Lauric | Aldehydic | Good | Clear | Bright |
| Aldehyde C-12 MNA | Aldehydic | Good | Clear | Bright |
| Aldehyde C14 | Fruity | Excellent | Clear | Moderate |
| (Undecalactone γ) | | | | |
| Aldehyde Mandarine | Aldehydic | ND | ND | ND |
| Allyl Amyl Glycolate | Fruity | Good | Slightly | Bright |
| | | | Haze | |
| Ambrinol | Amber | Good | Clear | Bright |
| Ambrofix | Amber | ND | ND | ND |
| Ambrox Super | Amber | None | Particles | Moderate |
| (Ambroxan) | | | | |
| Anethole | Anisic | Poor | Cloudy | Moderate |
| Anisic Aldehyde | Anisic | Good | Clear | Moderate |
| Anisyl Acetate | Anisic | Good | Clear | Moderate |
| Bacdanol | Woody | Good | Clear | Moderate |
| Benzyl Acetate | Floral | Excellent | Clear | Moderate |
| Caryophyllene | Spicy | Poor | Cloudy | Suppressed |
| Cashmeran (S51) | Musk | ND | ND | ND |
| Cashmeran (S55) | Musk | Good | Clear | Bright |
| Cetalox/Ambrox | Amber | ND | ND | ND |
| Cinnamalva | Spicy | Good | Clear | Bright |
| Citral | Citrus | Excellent | Clear | Bright |
| Clearwood | Woody | Moderate | Cloudy | Moderate |
| Cyclal C/Triplal | Green | Excellent | Clear | Bright |
| Damascenone | Floral | ND | ND | ND |
| Dibenzyl Ether | Earthy | Poor | Cloudy | Suppressed |
| Dihydro Eugenol | Spicy | Good | Clear | Bright |
| Dihydro Ionone Beta | Woody | ND | ND | ND |
| Dihydro Myrcenol | Citrus | Excellent | Clear | Bright |
| Dimetol | Herbal | Excellent | Clear | Bright |
| Ethyl Amyl Ketone | Herbal | ND | ND | ND |
| Ethyl Vanillin | Balsamic | Excellent | Clear | Bright |
| Ethylene Brassylate | Musk | Moderate | Cloudy | Bright |
| Eucalyptol | Herbal | Excellent | Clear | Bright |
| Evernyl | Earthy | Good | Clear | Moderate |
| Fennaldehyde | Anisic | ND | ND | ND |
| Fraistone | Fruity | ND | ND | ND |
| Fructone | Fruity | Good | Clear | Moderate |
| Galaxolide | Musk | Poor | Separation | Bright |
| Grisalva | Amber | Poor | Cloudy | Bright |
| Hedione | Floral | Good | Clear | Moderate |
| Heliotropine | Balsamic | Excellent | Clear | Bright |
| Cis-3-Hexenol | Green | Excellent | Clear | Bright |
| Cis-3-Hexenyl | Green | Poor | Cloudy | Moderate |
| Salicylate | | | | |
| Cis-3-Hexenyl Tiglate | Green | ND | ND | ND |
| Humus Ether | Earthy | Excellent | Clear | Bright |
| Iso E Super | Woody | Poor | Cloudy | Moderate |
| Isoamyl Acetate | Fruity | Excellent | Clear | Bright |
| Isobornyl Acetate | Balsamic | ND | ND | ND |
| Lime Oxide | Citrus | ND | ND | ND |
| Linalool | Floral | Good | Clear | Bright |
| Linalyl Acetate | Herbal | Moderate | Slightly | Bright |
| | | | Haze | |
| Methyl Cinnamate | Spicy | ND | ND | ND |
| Methyl Diantilis | Spicy | ND | ND | ND |
| Methyl Geosmin | Earthy | ND | ND | ND |
| Methyl Hexyl Ketone | Earthy | ND | ND | ND |
| Methyl Ionone Gamma | Floral | Poor | Cloudy | Moderate |
| Muscenone | Musk | Moderate | Cloudy | Moderate |
| Nootkatone | Citrus | Excellent | Clear | Bright |
| Olibanol | Balsamic | ND | ND | ND |
| Paradisamide | Green | Moderate | Clear | Bright |
| Precyclemone B | Citrus | ND | ND | ND |
| Terpinyl Acetate | Herbal | Moderate | Slightly | Bright |
| | | | Haze | |
| Toscanol | Anisic | ND | ND | ND |
| Vanillin | Balsamic | Excellent | Clear | Bright |
| Vertofix Coeur | Woody | Poor | Cloudy | Moderate |

TABLE 9 shows various characteristics of a 1% concentration of fragrance ingredients in bioderived 1,3-BG (bioBG) diluent that were determined. Specifically, the different fragrance ingredients were tested for solubility in bioderived 1,3-BG at 1%, and characterized as excellent, good, moderate, or poor. Clarity for each of the fragrance ingredients (at 1% in bioderived 1,3-BG) was determined by visual observations after compounding and blending the fragrance ingredient with bioderived 1,3-BG and identified as clear, slightly haze, cloudy, particles, or separation. Headspace for each of the fragrance ingredients (at 1% in bioderived 1,3-BG) was identified as bright, moderate, or suppressed. Not determinable (ND).

TABLE 9

| | Solu- | | |
|---|---|---|---|
| | bility | Clarity | Headspace |
| Fragrance Ingredient | 1% | 1% | 1% |
| Aldehyde C-10 | Good | Clear | Bright |
| Aldehyde C-11 Undecylenic | Moderate | Clear | Bright |
| Aldehyde C-12 Lauric | Excellent | Clear | Bright |
| Aldehyde C-12 MNA | Good | Clear | Bright |
| Aldehyde C14 | Excellent | Clear | Bright |
| (Undecalactone γ) | | | |
| Aldehyde Mandarine | Good | Clear | Bright |
| Allyl Amyl Glycolate | Good | Clear | Bright |
| Ambrinol | Good | Clear | Bright |
| Ambrofix | Poor | Cloudy | Bright |
| Ambrox ® Super (Ambroxan) | Good | Slightly Haze | Bright |
| Anethole | Moderate | Clear | Moderate |
| Anisic Aldehyde | Good | Clear | Moderate |
| Anisyl Acetate | Good | Clear | Moderate |
| Bacdanol | Good | Clear | Bright |
| Benzyl Acetate | Excellent | Clear | Moderate |
| Caryophyllene | Good | Slightly Haze | Bright |
| Cashmeran (S51) | Good | Clear | Bright |
| Cashmeran (S55) | ND | ND | ND |
| Cetalox/Ambrox | Good | Clear | Moderate |
| Cinnamalva | Good | Clear | Bright |
| Citral | Excellent | Clear | Bright |
| Clearwood | Moderate | Clear | Bright |
| Cyclal C/Triplal | Excellent | Clear | Bright |
| Damascenone | Excellent | Clear | Bright |
| Dibenzyl Ether | Moderate | Slightly Haze | Moderate |
| Dihydro Eugenol | Good | Clear | Bright |
| Dihydro Ionone Beta | ND | ND | ND |
| Dihydro Myrcenol | Excellent | Clear | Bright |
| Dimetol | Excellent | Clear | Bright |
| Ethyl Amyl Ketone | ND | ND | ND |
| Ethyl Vanillin | Excellent | Clear | Bright |
| Ethylene Brassylate | Moderate | Slightly Haze | Bright |
| Eucalyptol | Excellent | Clear | Bright |
| Evernyl | Good | Clear | Moderate |
| Fennaldehyde | Excellent | Clear | Bright |
| Fraistone | Poor | Separation | Bright |
| Fructone | Good | Clear | Bright |
| Galaxolide | Good | Slightly Haze | Bright |
| Grisalva | Moderate | Clear | Bright |
| Hedione | Good | Clear | Moderate |
| Heliotropine | Excellent | Clear | Bright |
| Cis-3-Hexenol | Excellent | Clear | Bright |
| Cis-3-Hexenyl Salicylate | Good | Clear | Bright |
| Cis-3-Hexenyl Tiglate | Poor | Cloudy | Moderate |
| Humus Ether | Excellent | Clear | Bright |
| Iso E Super | Moderate | Clear | Bright |
| Isoamyl Acetate | Excellent | Clear | Bright |
| Isobornyl Acetate | Excellent | Clear | Bright |
| Lime Oxide | Poor | Cloudy | Moderate |
| Linalool | Good | Clear | Bright |
| Linalyl Acetate | Moderate | Clear | Bright |
| Methyl Cinnamate | ND | ND | ND |
| Methyl Diantilis | ND | ND | ND |
| Methyl Geosmin | Excellent | Clear | Bright |
| Methyl Hexyl Ketone | Moderate | Clear | Bright |
| Methyl Ionone Gamma | Poor | Slightly Haze | Bright |
| Muscenone | Moderate | Clear | Bright |
| Nootkatone | Excellent | Clear | Bright |
| Olibanol | Poor | Cloudy | Suppressed |
| Paradisamide | Moderate | Clear | Bright |
| Precyclemone B | Good | Clear | Moderate |
| Terpinyl Acetate | Moderate | Clear | Bright |
| Toscanol | Excellent | Clear | Bright |
| Vanillin | Excellent | Clear | Bright |
| Vertofix Coeur | Good | Clear | Bright |

BioBG was found to be superior to other natural diluents, such as benzyl benzoate and isopropyl myristate, and comparable to dipropylene glycol for expression of fragrance or scents in those samples that have demonstrated a solubility of moderate to excellent.

TABLE 10

| | Diluent | | | |
|---|---|---|---|---|
| | Bioderived | Dipropylene | Benzyl | Isopropyl |
| | 1,3-BG | Glycol | Benzoate | Myristate |
| Avg Rating (1-10) | 9.37 | 9.47 | 8.65 | 8.52 |

For those fragrance ingredients observed as having a solubility of excellent, bioBG had a superior organoleptic rating compared to all of the other diluents for fragrance expression of the fragrance ingredients.

TABLE 11

| | Diluent | | | |
|---|---|---|---|---|
| | Bioderived | Dipropylene | Benzyl | Isopropyl |
| | 1,3-BG | Glycol | Benzoate | Myristate |
| Avg Rating (1-10) | 9.68 | 9.60 | 8.95 | 8.81 |

### Example 3: Technical Quality Analysis Testing

In addition to the odor changes observed by blotter testing, for each dilution, the viscosity was observed and found to be a workable. Where precipitation was observed, this suggested that the particular sample of fragrance ingredient was insoluble in diluent. Color changes were noted if there were any color variation resulting from the addition of fragrance ingredient in diluent. For select fragrance ingredients, skin testing was performed. Specifically, in the first round of testing, skin odor evaluation was performed on an interim basis for selective ingredients. Observed results showed good odor retention of the fragrance ingredient.

Additional observations specific to bioderived 1,3-butylene glycol (bioBG) indicated that its viscosity was very consistent among the different fragrance ingredients and each sample easily poured or pipetted. Compared to the other diluents (i.e., Dipropylene Glycol (DPG); Benzyl Benzoate (BB); and Isopropyl Myristate (IPM)), bioBG demonstrated exceptional visual clarity with a crystal clear appearance. Skin application of compositions comprising bioBG and each of the fragrance ingredients was found to be very comforting and each had a soft texture. None of the fragrance ingredients in bioBG had a slimy or heavy feeling.

### Example 4: Quantitative Analysis Testing

TABLE 12 shows various characteristics of a 1% concentration of fragrance ingredients in bioderived 1,3-Butylene Glycol (bioBG) diluent. In particular, the log P, water solubility at a temperature of 25° C., vapor pressure (i.e., higher the vapor pressure, the greater the proportion of the ingredient will be in a closed headspace at equilibrium), and flash point of each of the listed fragrance ingredients was evaluated. Also included is the molecular weight of each of the fragrance ingredients. Not determinable (ND).

TABLE 12

| | | | Water | Vapor | Flash |
|---|---|---|---|---|---|
| | Molecular | | Solubility | Pressure | Point |
| INGREDIENT | Weight | logP | (mg/L) | (mm hg) | (° C.) |
| Aldehyde C-10 | 156 | 3.97 | 43.520 | 0.207 | 82 |
| Aldehyde C-11 | 168 | 4.072 | 30.000 | 0.039 | 76 |
| Undecylenic | | | | | |
| Aldehyde C-12 | 184 | 4.989 | 4.649 | 0.034 | 116 |
| Lauric | | | | | |
| Aldehyde C-12 | 184 | 4.834 | 5.372 | 4.834 | 69 |
| MNA | | | | | |
| Aldehyde C14 | 184 | 2.961 | 128.300 | 0.003 | 160 |
| (Undecalactone | | | | | |
| γ) | | | | | |
| Aldehyde | ND | ND | ND | ND | ND |
| Mandarine | | | | | |
| Allyl Amyl | 186 | 2.7 | 510.700 | 0.04 | 93 |
| Glycolate | | | | | |
| Ambrinol | 194 | 3.785 | 134.300 | 0.0005 | 100 |
| Ambrofix | ND | ND | ND | ND | ND |
| Ambrox Super | 236 | 5.256 | 2.436 | 0.009 | 100 |
| (Ambroxan) | | | | | |
| Anethole | 148 | 3.39 | 111.000 | 0.069 | 110 |
| Anisic | 136 | 1.76 | 4290.000 | 1 | 116 |
| Aldehyde | | | | | |
| Anisyl Acetate | 180 | 1.887 | 782.900 | 0.021 | 135 |
| Bacdanol | 208 | 4.3 | 17.000 | 0.00028 | 199 |
| Benzyl Acetate | 150 | 1.96 | 3100.000 | 0.177 | 91 |
| Caryophyllene | 204 | 6.3 | 0.050 | 0.013 | 16 |
| Cashmeran | 206 | 4.5 | 5.937 | 0.003 | 94 |
| (S51) | | | | | |
| Cashmeran | ND | ND | ND | ND | ND |
| (S55) | | | | | |
| Cetalox/ | 236 | ND | ND | ND | ND |
| Ambrox | | | | | |
| Cinnamalva | 129 | 1.84 | 1067.000 | 0.023 | 94 |
| Citral | 152 | 3.45 | 1340.000 | 0.2 | 98 |
| Clearwood | 222 | 5.3 | 42.870 | 0.000278 | 100 |
| Cyclal C/ | 138 | 3 | 311.500 | 0.578 | 66 |
| Triplal | | | | | |
| Damascenone | 190 | ND | ND | ND | ND |
| Dibenzyl Ether | 198 | 3.31 | 40.000 | 0.00103 | 93 |
| Dihydro | 166 | 2.39 | 228.000 | 0.006 | 100 |
| Eugenol | | | | | |
| Dihydro | 194 | ND | ND | ND | ND |
| Ionone Beta | | | | | |
| Dihydro | 156 | 2.99 | 252.200 | 0.166 | 76 |
| Myrcenol | | | | | |
| Dimetol | 144 | 2.976 | 777.000 | 0.33 | 63 |
| Ethyl Amyl | 128 | ND | ND | ND | ND |
| Ketone | | | | | |
| Ethyl Vanillin | 166 | 1.58 | 2820.000 | 0.001 | 127 |
| Ethylene | 270 | 2.773 | 1.719 | 0 | 110 |
| Brassylate | | | | | |
| Eucalyptol | 154 | 2.74 | 3500.000 | 1.9 | 50 |
| Evernyl | 196 | 2.843 | 310.600 | 0.00001 | 183 |
| Fennaldehyde | 178 | ND | ND | ND | ND |
| Fraistone | 188 | ND | ND | ND | ND |
| Fructone | 174 | 0.982 | 100.000 | 0.219 | 93 |
| Galaxolide | 258 | 5.92 | 0.194 | 0.000414 | 104 |
| Grisalva | 264 | 6.58 | 0.020 | 0.005 | 94 |
| Hedione | 226 | 2.653 | 91.720 | 0.001 | 148 |
| Heliotropine | 150 | 1.05 | 3500.000 | 0.003 | 110 |
| Cis-3-Hexenol | 100 | 1.697 | 16000.000 | 1.039 | 44 |
| Cis-3-Hexenyl | 220 | 4.4 | 9.518 | 0.000246 | 110 |
| Salicylate | | | | | |
| Cis-3-Hexenyl | 182 | ND | ND | ND | ND |
| Tiglate | | | | | |
| Humus Ether | 169 | 2.931 | 305.100 | 0.049 | 98 |
| Iso E Super | 234 | 4.71 | 1.077 | 0.001 | 111 |
| Isoamyl | 130 | 2.26 | 2000.000 | 5.6 | 38 |
| Acetate | | | | | |
| Isobornyl | 196 | ND | ND | ND | ND |
| Acetate | | | | | |
| Lime Oxide | 56 | ND | ND | ND | ND |
| Linalool | 154 | 2.97 | 1590.000 | 0.016 | 75 |
| Linalyl Acetate | 196 | 3.93 | 20.120 | 0.116 | 94 |
| Methyl | 162 | ND | ND | ND | ND |
| Cinnamate | | | | | |
| Methyl | 182 | ND | ND | ND | ND |
| Diantilis | | | | | |
| Methyl | 185 | ND | ND | ND | ND |
| Geosmin | | | | | |
| Methyl Hexyl | 128 | ND | ND | ND | ND |
| Ketone | | | | | |
| Methyl Ionone | 206 | 4.315 | 2.980 | 0.006 | 110 |
| Gamma | | | | | |
| Muscenone | 236 | 4.88 | 0.346 | 0.1 | 100 |
| Nootkatone | 218 | 3.765 | 2.375 | 0.000358 | 110 |
| Olibanol | ND | ND | ND | ND | |
| Paradisamide | 219 | 3.295 | 46.430 | 0.0038 | 138 |
| Precyclemone | 206 | ND | ND | ND | ND |
| B | | | | | |
| Terpinyl | 196 | 3.59 | 18.970 | 0.058 | 100 |
| Acetate | | | | | |
| Toscanol | 162 | ND | ND | ND | ND |
| Vanillin | 152 | 1.21 | 6875.000 | 0.002 | 153 |
| Vertofix Coeur | 246 | 5.6 | 1.278 | 0.000084 | 100 |

A statistical regression analysis of 105 fragrance ingredients in BioBG showed log P and molecular weight that explain more than 99.9% of the variability in solubility with bioBG and the fragrance ingredients. BioBG also demonstrated a stronger solubility performance with more polar, synthetic fragrance ingredients, and less consistent solubility performance in less polar, natural fragrance ingredients.

### Example 5: Preservative-Boosting Performance

Minimum Inhibitory Concentration (MIC) testing has shown that bioBG plays a role in contamination prevention in solutions and formulations. BioBG demonstrated strong preservative properties in MIC testing, effectively inhibiting microbial growth at lower concentrations than several other diluents tested. BioBG completely inhibited microbial growth for the microorganisms at the highlighted concentrations (mg/ml) in TABLE 13. MIC tests indicated that bioBG had an increased ability to inhibit microbial growth compared to 1,3-Propylene Glycol (1,3-PG) and glycerin. It was found that bioBG plays a preservative role in view of the lower concentration values demonstrating a stronger anti-microbial efficacy.

TABLE 13

| | | | | Glycerin: | Glycerin: |
|---|---|---|---|---|---|
| | | 1,3- | Glyc- | 1,3-PG | BioBG |
| ORGANISM | BioBG | PG | erin | (1:1) | (1:1) |
| S. aureus | 225 | 300 | >350 | >350 | 300 |
| E. coli | 100 | 225 | >350 | >350 | 250 |
| P. aeruginosa | 175 | 200 | >350 | >350 | 200 |
| C. albicans | 225 | 250 | >350 | >350 | 300 |
| A. brasiliensis | >350 | >350 | >350 | >350 | >350 |

### Example 6: Life Cycle Assessment (LCA) of BioBG

A third-party, peer-reviewed Life Cycle Assessment (LCA) based on ISO 14040:2006 substantiated and measured the sustainability of natural, bioBG. It was shown that BioBG had a 50% reduction in greenhouse gases. This essentially translates to an annual reduction of nearly 100,000 tons of carbon dioxide equivalent (CO2e), and eliminates the use of 50,000 tons of acetaldehyde, which is a carcinogenic and mutagenic ingredient used in conventional production methods. BioBG was also determined, by ISO 16128:2016, to be a natural ingredient and 100% biobased. TABLE 14 shows different impact categories as assessed.

TABLE 14

| | | | | Percent |
|---|---|---|---|---|
| | | Petroleum- | | Improve- |
| Impact Category | Units | based BG | BioBG | ment |
| Global Warming | Kg CO₂ - | 3.78 | 1.86 | 50.8% |
| Impact | eq | | | |
| Primary Energy | MJ | 109 | 58.5 | 46.3% |
| Demand from Non- | | | | |
| Renewable Sources | | | | |

In view of the physical characteristics of bioBG, bioBG is a useful in fragrance compositions and applications. When applied to skin, bioBG (up to a concentration of 40%) has demonstrated non-irritating, non-sensitizing, and/or non-allergenic properties.

### Example 7: Repeated Insult Patch Test

In order to determine, whether a bioBG test material induced primary or cumulative irritation and/or allergic contact sensitization, repetitive epidermal contact was performed on fifty-nine (59) qualified subjects selected based on the inclusion and exclusion criteria outlined in TABLE 15. The subjects, both male and female, ranged in age from 21 years old to 79 years old, and fifty-six (56) subjects completed the study. The remaining subjects discontinued their participation for various reasons, none of which were related to the application of the test material.

TABLE 15

| Inclusion Criteria | Exclusion Criteria |
|---|---|
| Male and female subjects, ages 16 to 79 | Ill health |
| years old | |
| Absence of any visible skin disease which | Under a doctor's care or |
| might be confused with a skin reaction | taking medication(s) which |
| from the test material | could influence the |
| | outcome of the study |
| Prohibition of use of topical or systemic | Females who are pregnant |
| steroids and/or antihistamines for at least | or nursing |
| seven (7) days prior to study initiation | |
| Completion of a Medical History Form | A history of adverse reactions |
| and the understanding and signing of an | to cosmetics or other personal |
| Informed Consent Form | care products |
| Considered reliable and capable of | |
| following directions | |

Prior to the initiation of the study, each jar containing the test material received a N2 blanket conducted by the Analytical Department at Consumer Product Testing Company. The bioBG test material was prepared as a 40% dilution in water.

The upper back between the scapulae served as the treatment area. Approximately 0.2 ml of the test material, or an amount sufficient to cover the contact surface, was applied to the 1″×1″ absorbent pad portion of a clear adhesive dressing. This was then applied to the appropriate treatment site to form a semi-occlusive patch.

The evaluation criteria for erythema and additional dermal sequelae is presented in TABLE 16. Erythema was scored numerically according to the key in TABLE 16. If present, additional Dermal Sequelae were indicated by the appropriate letter code and a numerical value for severity was assigned.

TABLE 16

| | |
|---|---|
| Evaluation Criteria | Key |

| Value | Description | Letter Code | Description |
|---|---|---|---|
| 0 | No visible skin reaction | E | Edema |
| 0.5 | Barely perceptible | D | Dryness |
| 1 | Mild | S | Staining |
| 2 | Moderate | P | Papules |
| 3 | Marked | V | Vesicles |
| 4 | Severe | B | Bullae |
| | | U | Ulceration |
| | | Sp | Spreading |

### Induction Phase:

Patches were applied three (3) times per week (e.g., Monday, Wednesday, and Friday) for a total of nine (9) applications. The site was marked to ensure the continuity of patch application. Following supervised removal and scoring of the first Induction patch, participants were instructed to remove all subsequent Induction patches at home, twenty-four hours after application. The evaluation of this site was made again just prior to re-application. If a participant was unable to report for an assigned test day, one (1) makeup day was permitted. This day was added to the Induction period.

With the exception of the first supervised Induction Patch reading, if any test site exhibited a moderate (2-level) reaction during the Induction Phase, application was moved to an adjacent area. Applications were discontinued for the remainder of this test phase, if a moderate (2-level) reaction was observed on this new test site. Applications would also be discontinued if marked (3-level) or severe (4-level) reactivity was noted.

Rest periods consisted of one day following each Tuesday and Thursday removal, and two days following each Saturday removal.

### Challenge Phase:

Approximately two (2) weeks after the final Induction patch application, a Challenge patch was applied to a virgin test site adjacent to the original Induction patch site, following the same procedure described for the Induction Phase. The Challenge patch was removed and the site scored at the clinic on Day 1 and Day 3 post-application.

### Results:

There were no adverse events, no amendments, and no deviations. The results of each participate are presented in TABLE 17. Observations remained within normal limits throughout the test interval. TABLE 18 shows the demographics for each of the subject participants.

Test material, bioBG diluted to 40%, indicated no potential for dermal irritation or allergic contact sensitization.

TABLE 17

| | | Induction Phase | | | | | | | | | Virgin Challenge Site | |
|---|---|---|---|---|---|---|---|---|---|---|---|---|
| Subject # | Day 1* | 1 | 2 | 3 | 4 | 5 | 6 | 7 | 8 | 9 | Day 1* | Day 3 |
| 1 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 |
| 4 | —† | | | | | | | | | | | |
| 5 | —† | | | | | | | | | | | |
| 6 | 0 | 0 | 0 | 0 | 0 | 0 | 0 | 0 | 0 | 0 | 0 | 0 |
| 7 | 0 | 0 | 0 | 0 | 0 | 0 | 0 | 0 | 0 | 0 | 0 | 0 |
| 8 | 0 | 0 | 0 | 0 | 0 | 0 | 0 | 0 | 0 | 0 | 0 | 0 |
| 9 | 0 | 0 | 0 | 0 | 0 | 0 | 0 | 0 | 0 | 0 | 0 | 0 |
| 10 | —† | 0 | 0 | 0 | 0 | 0 | 0 | 0 | 0 | 0 | 0 | 0 |
| 11 | —† | 0 | 0 | 0 | 0 | 0 | 0 | 0 | 0 | 0 | 0 | 0 |
| 12 | 0 | 0 | 0 | 0 | 0 | 0 | 0 | 0 | 0 | 0 | 0 | 0 |
| 13 | —† | 0 | 0 | 0 | 0 | 0 | 0 | 0 | 0 | 0 | 0 | 0 |
| 14 | —† | 0 | 0 | 0 | 0 | 0 | 0 | 0 | 0 | 0 | 0 | 0 |
| 15 | 0 | 0 | 0 | 0 | 0 | 0 | 0 | 0 | 0 | 0 | 0 | 0 |
| 16 | 0 | 0 | 0 | 0 | 0 | 0 | 0 | 0 | 0 | 0 | 0 | 0 |
| 17 | 0 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0 | 0 |
| 19 | 0 | 0 | 0 | 0 | 0 | 0 | 0 | 0 | 0 | 0 | 0 | 0 |
| 20 | ----------------------- DID NOT COMPLETE STUDY ------------------------ | | | | | | | | | | | |
| 21 | 0 | 0 | 0 | 0 | 0 | 0 | 0 | 0 | 0 | 0 | 0 | 0 |
| 22 | 0 | 0 | 0 | 0 | 0 | 0 | 0 | 0 | 0 | 0 | 0 | 0 |
| 23 | 0 | 0 | 0 | 0 | 0 | 0 | 0 | 0 | 0 | 0 | 0 | 0 |
| 24 | 0 | 0 | 0 | 0 | 0 | 0 | 0 | 0 | 0 | 0 | 0 | 0 |
| 25 | 0 | 0 | 0 | 0 | 0 | 0 | 0 | 0 | 0 | 0 | 0 | 0 |
| 26 | 0 | 0 | 0 | 0 | 0 | 0 | 0 | 0 | 0 | 0 | 0 | 0 |
| 27 | 0 | 0 | 0 | 0 | 0 | 0 | 0 | 0 | 0 | 0 | 0 | 0 |
| 28 | ----------------------- DID NOT COMPLETE STUDY ------------------------ | | | | | | | | | | | |
| 29 | 0 | 0 | 0 | 0 | 0 | 0 | 0 | 0 | 0 | 0 | 0 | 0 |
| 30 | 0 | 0 | 0 | 0 | 0 | 0 | 0 | 0 | 0 | 0 | 0 | 0 |
| 31 | 0 | 0 | 0 | 0 | 0 | 0 | 0 | 0 | 0 | 0 | 0 | 0 |
| 32 | 0 | 0 | 0 | 0 | 0 | 0 | 0 | 0 | 0 | 0 | 0 | 0 |
| 33 | 0 | 0 | 0 | 0 | 0 | 0 | 0 | 0 | 0 | 0 | 0 | 0 |
| 34 | ----------------------- DID NOT COMPLETE STUDY ------------------------ | | | | | | | | | | | |
| 35 | 0 | 0 | 0 | 0 | 0 | 0 | 0 | 0 | 0 | 0 | 0 | 0 |
| 36 | 0 | 0 | 0 | 0 | 0 | 0 | 0 | 0 | 0 | 0 | 0 | 0 |
| 37 | 0 | 0 | 0 | 0 | 0 | 0 | 0 | 0 | 0 | 0 | 0 | 0 |
| 38 | 0 | 0 | 0 | 0 | 0 | 0 | 0 | 0 | 0 | 0 | 0 | 0 |
| 39 | 0 | 0 | 0 | 0 | 0 | 0 | 0 | 0 | 0 | 0 | 0 | 0 |
| 40 | 0 | 0 | 0 | 0 | 0 | 0 | 0 | 0 | 0 | 0 | 0 | 0 |
| 41 | 0 | 0 | 0 | 0 | 0 | 0 | 0 | 0 | 0 | 0 | 0 | 0 |
| 42 | 0 | 0 | 0 | 0 | 0 | 0 | 0 | 0 | 0 | 0 | 0 | 0 |
| 43 | 0 | 0 | 0 | 0 | 0 | 0 | 0 | 0 | 0 | 0 | 0 | 0 |
| 44 | 0 | 0 | 0 | 0 | 0 | 0 | 0 | 0 | 0 | 0 | 0 | 0 |
| 45 | 0 | 0 | 0 | 0 | 0 | 0 | 0 | 0 | 0 | 0 | 0 | 0 |
| 46 | 0 | 0 | 0 | 0 | 0 | 0 | 0 | 0 | 0 | 0 | 0 | 0 |
| 47 | 0 | 0 | 0 | 0 | 0 | 0 | 0 | 0 | 0 | 0 | 0 | 0 |
| 48 | 0 | 0 | 0 | 0 | 0 | 0 | 0 | 0 | 0 | 0 | 0 | 0 |
| 49 | 0 | 0.5 | 0 | 0 | 0 | 0 | 0 | 0 | 0 | 0 | 0 | 0 |
| 50 | 0 | 0 | 0 | 0 | 0 | 0 | 0 | 0 | 0 | 0 | 0 | 0 |
| 51 | 0 | 0 | 0 | 0 | 0 | 0 | 0 | 0 | 0 | 0 | 0 | 0 |
| 52 | 0 | 0 | 0 | 0 | 0 | 0 | 0 | 0 | 0 | 0 | 0 | 0 |
| 53 | 0 | 0 | 0 | 0 | 0 | 0 | 0 | 0 | 0 | 0 | 0 | 0 |
| 54 | 0 | 0 | 0 | 0 | 0 | 0 | 0 | 0 | 0 | 0 | 0 | 0 |
| 55 | 0 | 0 | 0 | 0 | 0 | 0 | 0 | 0 | 0 | 0 | 0 | 0 |
| 56 | 0 | 0 | 0 | 0 | 0 | 0 | 0 | 0 | 0 | 0 | 0 | 0 |
| 57 | 0 | 0 | 0 | 0 | 0 | 0 | 0 | 0 | 0 | 0 | 0 | 0 |
| 58 | 0 | 0 | 0 | 0 | 0 | 0 | 0 | 0 | 0 | 0 | 0 | 0 |
| 59 | 0 | 0 | 0 | 0 | 0 | 0 | 0 | 0 | 0 | 0 | 0 | 0 |

Day 1*= Supervised removal
†= Unsupervised removal due to inclement weather

TABLE 18

| Subject # | Age | Gender |
|---|---|---|
| 1 | 72 | M |
| 2 | 76 | F |
| 3 | 77 | F |
| 4 | 76 | M |
| 5 | 79 | M |
| 6 | 72 | M |
| 7 | 73 | F |
| 8 | 73 | M |
| 9 | 72 | M |
| 10 | 74 | F |
| 11 | 76 | F |
| 12 | 71 | F |
| 13 | 56 | M |
| 14 | 75 | F |
| 15 | 55 | M |
| 16 | 32 | F |
| 17 | 51 | M |
| 18 | 49 | F |
| 19 | 52 | F |
| 20 | 52 | F |
| 21 | 68 | M |
| 22 | 52 | F |
| 23 | 42 | F |
| 24 | 57 | F |
| 25 | 34 | M |
| 26 | 73 | F |
| 27 | 44 | F |
| 28 | 44 | F |
| 29 | 72 | M |
| 30 | 44 | F |
| 31 | 60 | F |
| 32 | 57 | F |
| 33 | 30 | M |
| 34 | 37 | F |
| 35 | 54 | F |
| 36 | 75 | F |
| 37 | 53 | F |
| 38 | 50 | M |
| 39 | 57 | F |
| 40 | 67 | M |
| 41 | 40 | F |
| 42 | 64 | F |
| 43 | 65 | F |
| 44 | 63 | F |
| 45 | 39 | F |
| 46 | 65 | M |
| 47 | 64 | F |
| 48 | 71 | M |
| 49 | 46 | F |
| 50 | 24 | M |
| 51 | 42 | F |
| 52 | 51 | M |
| 53 | 67 | M |
| 54 | 43 | F |
| 55 | 62 | F |
| 56 | 67 | M |
| 57 | 21 | F |
| 58 | 57 | F |
| 59 | 32 | M |

### Specific Embodiments

Non-limiting specific embodiments are described here, each of which is considered to be within the present disclosure.

As various changes can be made in the described subject matter without departing from the scope and spirit of the present disclosure, it is intended that all subject matter contained in the description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. A composition, comprising:
(a) a 1,3-butylene glycol (1,3-BG); and
(b) one or more fragrance ingredients,
wherein the components of (a) and (b) form a fragrance component, wherein the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur.

2. The composition of claim 1, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.

3. The composition of claim 1, wherein the composition comprises one or more fragrance ingredients is selected from the group consisting of: Aldehyde C-12 Lauric; Aldehyde C14 (Undecalactone γ); Benzyl Acetate; Citral; Cyclal C/Triplal; Damascenone; Dihydro Myrcenol; Dimetol; Ethyl Vanillin; Eucalyptol; Fennaldehyde; Heliotropine; Cis-3-Hexenol; Humus Ether; Isoamyl Acetate; Isobornyl Acetate; Methyl Geosmin; Nootkatone; Toscanol; and Vanillin.

4. The composition of claim 1, wherein the composition comprises one or more fragrance ingredients is selected from the group consisting of: Aldehyde C-10; Aldehyde C-12 Lauric; Aldehyde C-12 MNA; Aldehyde Mandarine; Allyl Amyl Glycolate; Ambrinol; Ambrox Super (Ambroxan); Anisic Aldehyde; Anisyl Acetate; Bacdanol; Caryophyllene; Cashmeran; Cetalox/Ambrox; Cinnamalva; Dihydro Eugenol; Evernyl; Fructone; Galaxolide; Hedione; Cis-3-Hexenyl Salicylate; Linalool; Precyclemone B; and Vertofix Coeur.

5. The composition of claim 1, wherein the composition comprises one or more fragrance ingredients is selected from the group consisting of: Aldehyde C-11 Undecylenic; Anethole; Clearwood; Dibenzyl Ether; Ethylene Brassylate; Grisalva; Iso E Super; Linalyl Acetate; Methyl Hexyl Ketone; Muscenone; Paradisamide; and Terpinyl Acetate.

6. The composition of claim 1, wherein the composition comprises one or more fragrance ingredients is selected from the group consisting of: Aldehyde C-12 Lauric; Aldehyde C-12 MNA; Aldehyde C14 (Undecalactone γ); Ambrinol; Anisic Aldehyde; Anisyl Acetate; Bacdanol; Benzyl Acetate; Cashmeran; Cinnamalva; Citral; Cyclal C/Triplal; Dihydro Eugenol; Dihydro Myrcenol; Dimetol; Ethyl Vanillin; Eucalyptol; Evernyl; Fructone; Hedione; Heliotropine; Cis-3-Hexenol; Humus Ether; Isoamyl Acetate; Linalool; Nootkatone; Paradisamide; and Vanillin.

7. The composition of claim 1, wherein the composition comprises one or more fragrance ingredients is selected from the group consisting of: Aldehyde C-10; Aldehyde C-11 Undecylenic; Allyl Amyl Glycolate; Linalyl Acetate; and Terpinyl Acetate.

8. The composition of claim 1, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Anethole; Anisic Aldehyde; Anisyl Acetate; Bacdanol; Benzyl Acetate; Cashmeran; Cetalox/Ambrox; Cinnamalva; Citral; Clearwood; Cyclal C/Triplal; Damascenone; Dihydro Eugenol; Dihydro Myrcenol; Dimetol; Ethyl Vanillin; Eucalyptol; Evernyl; Fennaldehyde; Fructone; Grisalva; Hedione; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.

9. The composition of claim 1, wherein the composition comprises one or more fragrance ingredients is selected from the group consisting of: Ambrox Super (Ambroxan); Dibenzyl Ether; Methyl Ionone Gamma; Ethylene Brassylate; and Galaxolide; Caryophyllene.

10. The composition of claim 1, wherein the composition comprises one or more fragrance ingredients is selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Grisalva; Galaxolide; Heliotropine; Cis-3-Hexenol; Humus Ether; Isoamyl Acetate; Linalool; Linalyl Acetate; Nootkatone; Paradisamide; Terpinyl Acetate; and Vanillin.

11. The composition of claim 1, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur.

12. The composition of claim 1, wherein the composition comprises one or more fragrance ingredients is selected from the group consisting of: Aldehyde C14 (Undecalactone γ); Ambrox Super (Ambroxan); Anethole; Anisic Aldehyde; Anisyl Acetate; Bacdanol; Benzyl Acetate; Clearwood; Evernyl; Fructone; Hedione; Cis-3-Hexenyl Salicylate; Iso E Super; Methyl Ionone Gamma; Muscenone; and Vertofix Coeur.

13. The composition of claim 1, wherein the composition comprises one or more fragrance ingredients is selected from the group consisting of: Anethole; Anisic Aldehyde; Anisyl Acetate; Benzyl Acetate; Cetalox/Ambrox; Dibenzyl Ether; Evernyl; Hedione; Cis-3-Hexenyl Tiglate; Lime Oxide; and Precyclemone B.

14. The composition according to claim 1, wherein the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Cis-3-Hexenyl Tiglate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Lime Oxide; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix CoeurEvernyl.

15. The composition according to claim 1, wherein the one or more fragrance ingredients is selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Galaxolide; Grisalva; Heliotropine; Cis-3-Hexenol; Humus Ether; Isoamyl Acetate; Linalool; Linalyl Acetate; Nootkatone; Paradisamide; Terpinyl Acetate; and Vanillin.

16. The composition according to claim 1, wherein the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur.

17. The composition of claim 1, wherein the composition comprises one or more fragrance ingredients is selected from the group consisting of: Aldehyde C-12 Lauric; Aldehyde C-12 MNA; Aldehyde C14 (Undecalactone γ); Allyl Amyl Glycolate; Ambrinol; Anisic Aldehyde; Anisyl Acetate; Cashmeran; Cinnamalva; Citral; Dihydro Eugenol; Dimetol; Eucalyptol; Heliotropine; Cis-3-Hexenol; Humus Ether; Linalool; Nootkatone; and Vanillin.

18. The composition of claim 1, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Allyl Amyl Glycolate; Ambrinol; Ambrox Super (Ambroxan); Anethole; Anisic Aldehyde; Anisyl Acetate; Bacdanol; Benzyl Acetate; Cashmeran; Cinnamalva; Citral; Clearwood; Cyclal C/Triplal; Dihydro Eugenol; Dihydro Myrcenol; Dimetol; Ethyl Vanillin; Ethylene Brassylate; Eucalyptol; Evernyl; Galaxolide; Grisalva; Hedione; Heliotropine; Cis-3-Hexenol; Humus Ether; Iso E Super; Isoamyl Acetate; Linalool; Linalyl Acetate; Methyl Ionone Gamma; Muscenone; Nootkatone; Paradisamide; Terpinyl Acetate; Vanillin; and Vertofix Coeur.

19. The composition according to claim 1, wherein the one or more fragrance ingredients is selected from the group consisting of: Aldehyde C14 (Undecalactone γ); Citral; Dimetol; Nootkatone; Ambrinol; Aldehyde C-12 Lauric; and Aldehyde C-12 MNA.

20. The composition of claim 1, wherein the composition comprises one or more fragrance ingredients, wherein the fragrance ingredient is not selected from the group consisting of: Ambrox Super (Ambroxan); Anethole; Benzyl Acetate; Caryophyllene; Clearwood; Dibenzyl Ether; Ethylene Brassylate; Evernyl; Fructone; Galaxolide; Grisalva; Hedione; Cis-3-Hexenyl Salicylate; Iso E Super; Linalyl Acetate; Methyl Ionone Gamma; Muscenone; Paradisamide; Terpinyl Acetate; and Vertofix Coeur.

21. The composition of claim 1, wherein the composition comprises one or more fragrance ingredients, wherein the fragrance ingredient is not selected from the group consisting of: Ambrox Super (Ambroxan); Anethole; Caryophyllene; Dibenzyl Ether; Galaxolide; Grisalva; Cis-3-Hexenyl Salicylate; Iso E Super; Methyl Ionone Gamma; and Vertofix Coeur.

22. The composition of claim 1, wherein the composition comprises one or more fragrance ingredients, wherein the fragrance ingredient is not selected from the group consisting of: Ambrox Super (Ambroxan); Anethole; Caryophyllene; Clearwood; Dibenzyl Ether; Ethylene Brassylate; Galaxolide; Grisalva; Cis-3-Hexenyl Salicylate; Iso E Super; Methyl Ionone Gamma; Muscenone; and Vertofix Coeur.

23. The composition of claim 1, wherein the composition comprises one or more fragrance ingredients, wherein the fragrance ingredient is not selected from the group consisting of: Aldehyde Mandarine; Ambrofix; Cashmeran; Cetalox/Ambrox; Damascenone; Dihydro Ionone Beta; Ethyl Amyl Ketone; Fennaldehyde; Fraistone; Cis-3-Hexenyl Tiglate; Isobornyl Acetate; Lime Oxide; Methyl Cinnamate; Methyl Diantilis; Methyl Geosmin; Methyl Hexyl Ketone; Olibanol; Precyclemone B; and Toscanol.

24. The composition of claim 1, wherein the composition comprises one or more fragrance ingredients, wherein the fragrance ingredient is not selected from the group consisting of: Caryophyllene; and Dibenzyl Ether.

25. The composition of claim 1, wherein the composition comprises one or more fragrance ingredients, wherein the fragrance ingredient is not selected from the group consisting of: Ambrofix; Fraistone; Cis-3-Hexenyl Tiglate; Lime Oxide; Methyl Ionone Gamma; and Olibanol.

26. The composition of claim 1, wherein the composition comprises one or more fragrance ingredients, wherein the fragrance ingredient is not Methyl Ionone Gamma or Olibanol.

27. The composition of claim 1, wherein the composition comprises one or more fragrance ingredients, wherein the fragrance ingredient is not selected from the group consisting of: Ambrofix; Fraistone; Cis-3-Hexenyl Tiglate; Lime Oxide; and Olibanol.

28. The composition of claim 1, wherein the composition comprises one or more fragrance ingredients, wherein the fragrance ingredient is not selected from the group consisting of: Ethyl Amyl Ketone; Cashmeran; Dihydro Ionone Beta; Methyl Cinnamate; and Methyl Diantilis.

29. The composition of claim 1, wherein the composition comprises one or more fragrance ingredients, wherein the fragrance ingredient is not selected from the group consisting of: Caryophyllene; Dibenzyl Ether; Fructone; and Cis-3-Hexenyl Salicylate.

30. The composition according to claim 1, wherein the 1,3-butylene glycol (1,3-BG) is selected from the group consisting of: 1,3-BG, bioBG, or a mixture of 1,3-BG and bioBG.

31. The composition according to claim 1, wherein the 1,3-butylene glycol (1,3-BG) comprises bioBG.

32. The composition according to claim 1, wherein the 1,3-BG is selected from the group consisting of: (R)-1,3-BG; (S)-1,3-BG; and a mixture of (R)-1,3-BG and (S)-1,3-BG.

33. The composition according to claim 32, wherein the composition comprises 1,3-BG, wherein the 1,3-BG is (R)-1,3-BG.

34. The composition according to claim 32, wherein the composition comprises 1,3-BG, wherein the 1,3-BG is (S)-1,3-BG.

35. The composition according to claim 32, wherein the composition comprises 1,3-BG, wherein 1,3-BG is a mixture of (R)-1,3-BG and (S)-1,3-BG.

36. The composition according to claim 32, wherein the mixture comprises 75% or greater of (R)-1,3-BG.

37. The composition according to claim 32, wherein the mixture comprises 25% or less of (S)-1,3-BG.

38. The composition according to claim 1, wherein 1,3-BG is a microbial growth inhibitor.

39. The composition according to claim 1, wherein 1,3-BG is a preservative.

40. The composition according to claim 1, wherein each of the one or more fragrance ingredients has a molecular weight of or greater than 90 g/mol.

41. The composition according to claim 1, wherein each of the one or more fragrance ingredients has a molecular weight of or less than 270 g/mol.

42. The composition according to claim 1, wherein each of the one or more fragrance ingredients has a molecular weight of 90 g/mol to 270 g/mol.

43. The composition according to claim 1, wherein each of the one or more fragrance ingredients has a log P value of or greater than 0.5.

44. The composition according to claim 1, wherein each of the one or more fragrance ingredients has a log P value of or less than 7.

45. The composition according to claim 1, wherein each of the one or more fragrance ingredients has a log P value of 0.5 to 7.

46. The composition according to claim 1, wherein the composition comprises more polar fragrance ingredients than non-polar fragrance ingredients.

47. The composition according to claim 1, wherein each of the one or more fragrance ingredients is polar.

48. The composition according to claim 1, wherein the composition comprises one or more fragrance ingredients selected from the group consisting of: synthetic, natural, and a mixture of synthetic and natural.

49. The composition according to claim 46, wherein each of the one or more fragrance ingredients is synthetic.

50. The composition according to claim 1, further comprising one or more diluents.

51. The composition according to claim 50, wherein the one or more diluents is selected from the group consisting of: 1,3-Butylene Glycol (1,3-BG); bioderived 1,3-Butylene Glycol (bioBG); Dipropylene Glycol (DPG); Isopropyl Myristate (IPM); Benzyl Benzoate (BB); 1,3-Propylene Glycol (1,3-PG); glycerin; and any combination thereof.

52. The composition according to claim 1, wherein the composition is selected from the group of properties consisting of: non-irritating, non-sensitizing, non-allergenic, non-greasy, and any combination thereof.

53. The composition according to claim 1, wherein the composition comprises a fragrance component comprising one or more fragrance ingredients present in an amount of or greater than 0.01% by weight.

54. The composition according to claim 1, wherein the fragrance component comprises one or more fragrance ingredients present in an amount of or less than 60% by weight.

55. The composition according to claim 1, wherein the fragrance component comprises one or more fragrance ingredients present in an amount of 0.01% by weight to 60% by weight.

56. A method, comprising applying to skin of a subject in need thereof, a composition of any one of claims 1 to 55, wherein the composition is a fragrance composition.

57. The method according to claim 56, wherein the one or more fragrance ingredients is selected from the group of characteristics consisting of: polar, synthetic, excellent solubility, clear clarity, and moderate to bright headspace, and combinations thereof.

58. Use of 1,3-butylene glycol (1,3-BG) as a diluent in a fragrance component of a composition, wherein the fragrance component further comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Citral; Clearwood; Cyclal C/Triplal; Damascenone; Dibenzyl Ether; Dihydro Eugenol; Dihydro Ionone Beta; Dihydro Myrcenol; Dimetol; Ethyl Amyl Ketone; Ethyl Vanillin; Ethylene Brassylate; Eucalyptol; Evernyl; Fennaldehyde; Fraistone; Fructone; Galaxolide; Grisalva; Hedione; Heliotropine; Cis-3-Hexenol; Cis-3-Hexenyl Salicylate; Cis-3-Hexenyl Tiglate; Humus Ether; Iso E Super; Isoamyl Acetate; Isobornyl Acetate; Lime Oxide; Linalool; Linalyl Acetate; Methyl Cinnamate; Methyl Diantilis; Methyl Geosmin; Methyl Hexyl Ketone; Methyl Ionone Gamma; Muscenone; Nootkatone; Olibanol; Paradisamide; Precyclemone B; Terpinyl Acetate; Toscanol; Vanillin; and Vertofix Coeur.

59. The use according to claim 58, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Galaxolide; Grisalva; Hedione; Heliotropine; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.

60. The use according to claim 58, wherein the composition comprises one or more fragrance ingredients is selected from the group consisting of: Aldehyde C-12 Lauric; Aldehyde C14 (Undecalactone γ); Benzyl Acetate; Citral; Cyclal C/Triplal; Damascenone; Dihydro Myrcenol; Dimetol; Ethyl Vanillin; Eucalyptol; Fennaldehyde; Heliotropine; Cis-3-Hexenol; Humus Ether; Isoamyl Acetate; Isobornyl Acetate; Methyl Geosmin; Nootkatone; Toscanol; and Vanillin.

61. The composition of claim 1, wherein the composition comprises one or more fragrance ingredients is selected from the group consisting of: Aldehyde C-10; Aldehyde C-12 Lauric; Aldehyde C-12 MNA; Aldehyde Mandarine; Allyl Amyl Glycolate; Ambrinol; Ambrox Super (Ambroxan); Anisic Aldehyde; Anisyl Acetate; Bacdanol; Caryophyllene; Cashmeran; Cetalox/Ambrox; Cinnamalva; Dihydro Eugenol; Evernyl; Fructone; Galaxolide; Hedione; Cis-3-Hexenyl Salicylate; Linalool; Precyclemone B; and Vertofix Coeur.

62. The composition of claim 1, wherein the composition comprises one or more fragrance ingredients is selected from the group consisting of: Aldehyde C-11 Undecylenic; Anethole; Clearwood; Dibenzyl Ether; Ethylene Brassylate; Grisalva; Iso E Super; Linalyl Acetate; Methyl Hexyl Ketone; Muscenone; Paradisamide; and Terpinyl Acetate.

63. The use according to claim 58, wherein the composition comprises one or more fragrance ingredients selected from the group consisting of: Aldehyde C-10; Aldehyde C-11 Undecylenic; Allyl Amyl Glycolate; Linalyl Acetate; and Terpinyl Acetate.

64. The use according to claim 58, wherein the composition comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Anethole; Anisic Aldehyde; Anisyl Acetate; Bacdanol; Benzyl Acetate; Cashmeran; Cetalox/Ambrox; Cinnamalva; Citral; Clearwood; Cyclal C/Triplal; Damascenone; Dihydro Eugenol; Dihydro Myrcenol; Dimetol; Ethyl Vanillin; Eucalyptol; Evernyl; Fennaldehyde; Fructone; Grisalva; Hedione; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur.

65. The use according to claim 58, wherein the composition comprises one or more fragrance ingredients selected from the group consisting of: Ambrox Super (Ambroxan); Caryophyllene Dibenzyl Ether; Methyl Ionone Gamma; Ethylene Brassylate; and Galaxolide.

66. The use according to claim 58, wherein the composition comprises one or more fragrance ingredients selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Grisalva; Galaxolide; Heliotropine; Cis-3-Hexenol; Humus Ether; Isoamyl Acetate; Linalool; Linalyl Acetate; Nootkatone; Paradisamide; Terpinyl Acetate; and Vanillin.

67. The use according to claim 58, wherein the composition comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur.

68. The use according to claim 58, wherein the composition comprises one or more fragrance ingredients selected from the group consisting of: Aldehyde C14 (Undecalactone γ); Ambrox Super (Ambroxan); Anethole; Anisic Aldehyde; Anisyl Acetate; Bacdanol; Benzyl Acetate; Clearwood; Evernyl; Fructone; Hedione; Cis-3-Hexenyl Salicylate; Iso E Super; Methyl Ionone Gamma; Muscenone; and Vertofix Coeur.

69. The use according to claim 58, wherein the composition comprises one or more fragrance ingredients selected from the group consisting of: Anethole; Anisic Aldehyde; Anisyl Acetate; Benzyl Acetate; Cetalox/Ambrox; Dibenzyl Ether; Evernyl; Hedione; Cis-3-Hexenyl Tiglate; Lime Oxide; and Precyclemone B.

70. The use according to claim 58, wherein the composition comprises one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Anethole; Anisic Aldehyde; Anisyl Acetate; Bacdanol; Benzyl Acetate; Caryophyllene; Cashmeran; Cetalox/Ambrox; Cinnamalva; Cis-3-Hexenol; Cis-3-Hexenyl Salicylate; Cis-3-Hexenyl Tiglate; Citral; Clearwood; Cyclal C/Triplal; Damascenone; Dibenzyl Ether; Dihydro Eugenol; Dihydro Myrcenol; Dimetol; Ethyl Vanillin; Ethylene Brassylate; Eucalyptol; Evernyl; Fennaldehyde; Fructone; Fennaldehyde; Galaxolide; Grisalva; Hedione; Heliotropine; Cis-3-Hexenol; Cis-3-Hexenyl Salicylate; Humus Ether; Iso E Super; Isoamyl Acetate; Isobornyl Acetate; Lime Oxide; Linalool; Linalyl Acetate; Methyl Geosmin; Methyl Hexyl Ketone; Muscenone; Nootkatone; Paradisamide; Precyclemone B; Terpinyl Acetate; Toscanol; Vanillin; and Vertofix Coeur; Isobornyl Acetate; Methyl Geosmin; Toscanol; Aldehyde Mandarine; Methyl Hexyl Ketone; Ambrofix; Anethole; Anisic Aldehyde; Anisyl Acetate; Benzyl Acetate; Dibenzyl Ether; Evernyl; Cetalox/Ambrox; Evernyl; Hedione; Lime Oxide; Precyclemone B; and Cis-3-Hexenyl Tiglate.

71. The composition according to claim 1, wherein the one or more fragrance ingredients is selected from the group consisting of: Aldehyde C-10; Aldehyde C-11 Undecylenic; Aldehyde C-12 Lauric; Aldehyde C-12 MNA; Allyl Amyl Glycolate; Ambrinol; Cashmeran; Cinnamalva; Citral; Cyclal C/Triplal; Dihydro Eugenol; Dihydro Myrcenol; Dimetol; Ethyl Vanillin; Ethylene Brassylate; Eucalyptol; Galaxolide; Grisalva; Heliotropine; Cis-3-Hexenol; Humus Ether; Isoamyl Acetate; Linalool; Linalyl Acetate; Nootkatone; Paradisamide; Terpinyl Acetate; and Vanillin.

72. The composition according to claim 1, wherein the one or more fragrance ingredients is selected from the group consisting of: Aldehyde C-10; Aldehyde C-11 Undecylenic; Aldehyde C-12 Lauric; Aldehyde C-12 MNA; Aldehyde C14 (Undecalactone γ); Aldehyde Mandarine; Allyl Amyl Glycolate; Ambrinol; Ambrofix; Ambrox Super (Ambroxan); Bacdanol; Caryophyllene; Cashmeran; Cinnamalva; Citral; Clearwood; Cyclal C/Triplal; Damascenone; Dihydro Eugenol; Dihydro Myrcenol; Dimetol; Ethyl Vanillin; Ethylene Brassylate; Eucalyptol; Fennaldehyde; Fraistone; Fructone; Galaxolide; Grisalva; Heliotropine; Cis-3-Hexenol; Cis-3-Hexenyl Salicylate; Humus Ether; Iso E Super; Isoamyl Acetate; Isobornyl Acetate; Linalool; Linalyl Acetate; Methyl Geosmin; Methyl Hexyl Ketone; Methyl Ionone Gamma; Muscenone; Nootkatone; Paradisamide; Terpinyl Acetate; Toscanol; Vanillin; and Vertofix Coeur.

73. The use according to claim 58, wherein the composition comprises one or more fragrance ingredients selected from the group consisting of: Aldehyde C-12 Lauric; Aldehyde C-12 MNA; Aldehyde C14 (Undecalactone γ); Allyl Amyl Glycolate; Ambrinol; Anisic Aldehyde; Anisyl Acetate; Cashmeran; Cinnamalva; Citral; Dihydro Eugenol; Dimetol; Eucalyptol; Heliotropine; Cis-3-Hexenol; Humus Ether; Linalool; Nootkatone; and Vanillin.

74. The use according to claim 58, wherein the composition comprises one or more fragrance ingredients selected from the group consisting of: Aldehyde C-10; Aldehyde C-11 Undecylenic; Aldehyde C-12 Lauric; Aldehyde C-12 MNA; Aldehyde C14 (Undecalactone γ); Allyl Amyl Glycolate; Ambrinol; Ambrox Super (Ambroxan); Anethole; Anisic Aldehyde; Anisyl Acetate; Bacdanol; Benzyl Acetate; Cashmeran; Cinnamalva; Citral; Clearwood; Cyclal C/Triplal; Dihydro Eugenol; Dihydro Myrcenol; Dimetol; Ethyl Vanillin; Ethylene Brassylate; Eucalyptol; Evernyl; Galaxolide; Grisalva; Hedione; Heliotropine; Cis-3-Hexenol; Humus Ether; Iso E Super; Isoamyl Acetate; Linalool; Linalyl Acetate; Methyl Ionone Gamma; Muscenone; Nootkatone; Paradisamide; Terpinyl Acetate; Vanillin; and Vertofix Coeur.

75. The use according to claim 58, wherein the composition comprises one or more fragrance ingredients selected from the group consisting of: Aldehyde C14 (Undecalactone γ); Citral; Dimetol; Nootkatone; Ambrinol; Aldehyde C-12 Lauric; and Aldehyde C-12 MNA.

76. The use according to claim 58, wherein the composition comprises one or more fragrance ingredients, wherein the fragrance ingredient is not selected from the group consisting of: Ambrox Super (Ambroxan); Anethole; Benzyl Acetate; Caryophyllene; Clearwood; Dibenzyl Ether; Ethylene Brassylate; Evernyl; Fructone; Galaxolide; Grisalva; Hedione; Cis-3-Hexenyl Salicylate; Iso E Super; Linalyl Acetate; Methyl Ionone Gamma; Muscenone; Paradisamide; Terpinyl Acetate; and Vertofix Coeur.

77. The use according to claim 58, wherein the composition comprises one or more fragrance ingredients, wherein the fragrance ingredient is not selected from the group consisting of: Ambrox Super (Ambroxan); Anethole; Caryophyllene; Dibenzyl Ether; Galaxolide; Grisalva; Cis-3-Hexenyl Salicylate; Iso E Super; Methyl Ionone Gamma; and Vertofix Coeur.

78. The use according to claim 58, wherein the composition comprises one or more fragrance ingredients, wherein the fragrance ingredient is not selected from the group consisting of: Ambrox Super (Ambroxan); Anethole; Caryophyllene; Clearwood; Dibenzyl Ether; Ethylene Brassylate; Galaxolide; Grisalva; Cis-3-Hexenyl Salicylate; Iso E Super; Methyl Ionone Gamma; Muscenone; and Vertofix Coeur.

79. The use according to claim 58, wherein the composition comprises one or more fragrance ingredients, wherein the fragrance ingredient is not selected from the group consisting of: Aldehyde Mandarine; Ambrofix; Cashmeran; Cetalox/Ambrox; Damascenone; Dihydro Ionone Beta; Ethyl Amyl Ketone; Fennaldehyde; Fraistone; Cis-3-Hexenyl Tiglate; Isobornyl Acetate; Lime Oxide; Methyl Cinnamate; Methyl Diantilis; Methyl Geosmin; Methyl Hexyl Ketone; Olibanol; Precyclemone B; and Toscanol.

80. The use according to claim 58, wherein the composition comprises one or more fragrance ingredients, wherein the fragrance ingredient is not selected from the group consisting of: Caryophyllene; and Dibenzyl Ether.

81. The use according to claim 58, wherein the composition comprises one or more fragrance ingredients, wherein the fragrance ingredient is not selected from the group consisting of: Ambrofix; Fraistone; Cis-3-Hexenyl Tiglate; Lime Oxide; Methyl Ionone Gamma; and Olibanol.

82. The use according to claim 58, wherein the composition comprises one or more fragrance ingredients, wherein the fragrance ingredient is not selected from the group consisting of: Methyl Ionone Gamma or Olibanol.

83. The use according to claim 58, wherein the composition comprises one or more fragrance ingredients, wherein the fragrance ingredient is not selected from the group consisting of: Ambrofix; Fraistone; Cis-3-Hexenyl Tiglate; Lime Oxide; and Olibanol.

84. The use according to claim 58, wherein the composition comprises one or more fragrance ingredients, wherein the fragrance ingredient is not selected from the group consisting of: Ethyl Amyl Ketone; Cashmeran; Dihydro Ionone Beta; Methyl Cinnamate; and Methyl Diantilis.

85. The use according to claim 58, wherein the composition comprises one or more fragrance ingredients, wherein the fragrance ingredient is not selected from the group consisting of: Caryophyllene; Dibenzyl Ether; Fructone; and Cis-3-Hexenyl Salicylate.

86. The use according to claim 58, wherein the 1,3-butylene glycol (1,3-BG) is selected from the group consisting of: 1,3-BG, bioBG, or a mixture of 1,3-BG and bioBG.

87. The use according to claim 58, wherein the 1,3-butylene glycol (1,3-BG) comprises bioBG.

88. The use according to claim 58, wherein the 1,3-butylene glycol is selected from the group consisting of: (R)-1,3-BG; (S)-1,3-BG; and a mixture of (R)-1,3-BG and (S)-1,3-BG.

89. The use according to claim 58, wherein the 1,3-BG is (R)-1,3-BG.

90. The use according to claim 58, wherein the 1,3-BG is (S)-1,3-BG.

91. The use according to claim 58, wherein the 1,3-BG is a mixture of (R)-1,3-BG and (S)-1,3-BG.

92. The use according to claim 88, wherein the mixture comprises 75% or greater of (R)-1,3-BG.

93. The use according to claim 88, wherein the mixture comprises 25% or less of (S)-1,3-BG.

94. The use according to claim 58, wherein 1,3-BG is a microbial growth inhibitor.

95. The use according to claim 58, wherein 1,3-BG is a preservative.

96. The use according to claim 58, wherein each of the one or more fragrance ingredients has a molecular weight of or greater than 90 g/mol.

97. The use according to claim 58, wherein each of the one or more fragrance ingredients has a molecular weight of or less than 270 g/mol.

98. The use according to claim 58, wherein each of the one or more fragrance ingredients has a molecular weight of 100 g/mol to 270 g/mol.

99. The use according to claim 58, wherein each of the one or more fragrance ingredients has a log P value of or greater than 0.5.

100. The use according to claim 58, wherein each of the one or more fragrance ingredients has a log P value of or less than 7.

101. The use according to claim 58, wherein each of the one or more fragrance ingredients has a log P value of 0.5 to 7.

102. The use according to claim 58, wherein the composition comprises more polar fragrance ingredients than non-polar fragrance ingredients.

103. The use according to claim 58, wherein each of or at least one of the one or more fragrance ingredients is polar.

104. The use according to claim 58, wherein the composition comprises one or more fragrance ingredients selected from the group consisting of: synthetic, natural, and a mixture of synthetic and natural.

105. The use according to claim 58, wherein each of the one or more fragrance ingredients is synthetic.

106. The use according to claim 58, further comprising one or more diluents.

107. The use according to claim 106, wherein the one or more diluents is selected from the group consisting of: 1,3-Butylene Glycol (1,3-BG); bioderived 1,3-Butylene Glycol (bioBG); Dipropylene Glycol (DPG); Isopropyl Myristate (IPM); Benzyl Benzoate (BB); 1,3-Propylene Glycol (1,3-PG); glycerin; and any combinations thereof.

108. The use according to claim 58, wherein the composition is selected from the group of properties consisting of: non-irritating, non-sensitizing, non-allergenic, non-greasy, and any combination thereof.

109. The use according to claim 58, wherein the composition comprises a fragrance component comprising one or more fragrance ingredients present in an amount of or greater than 0.01% by weight.

110. The use according to claim 58, wherein the fragrance component comprises one or more fragrance ingredients present in an amount of or less than 60% by weight.

111. The use according to claim 58, wherein the fragrance component comprises one or more fragrance ingredients present in an amount of 0.01% by weight to 60% by weight.
